(19) United States
(12) Patent Application Publication
(10) Pub. No.: US 2024/0301042 A1
(43) Pub. Date: Sep. 12, 2024

(54) SELECTIVE TARGETING OF OLIGOMERIC B-AMYLOID

(71) Applicant: ARIZONA BOARD OF REGENTS ON BEHALF OF ARIZONA STATE UNIVERSITY, Scottsdale, AZ (US)

(72) Inventors: Ping HE, Chandler, AZ (US); Michael SIERKS, Ft. McDowell, AZ (US)

(21) Appl. No.: 18/283,697

(22) PCT Filed: Mar. 23, 2022
(86) PCT No.: PCT/US2022/021567

§ 371 Date: Sep. 22, 2023

Related U.S. Application Data
(60) Provisional application No. 63/164,979, filed on Mar. 23, 2021.

(51) Int. Cl. C07K 16/18; A61K 39/00; A61P 25/28

(52) U.S. Cl. CPC C07K 16/18; A61P 25/28; A61K 2039/505; C07K 2317/622; C07K 2317/74; C07K 2319/10

(57) ABSTRACT

The invention provides methods and compositions for treating or ameliorating Alzheimer's disease. The invention provides agents that reduce Aβ inclusions and plaque accumulation, inhibit dendrite spine loss, and restore neurogenesis.

### RELATED APPLICATIONS AND INCORPORATION BY REFERENCE

This application claims priority to U.S. Patent Application Ser. No. 63/164,979 filed Mar. 23, 2021.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FEDERAL FUNDING LEGEND

This invention was made with government support under S10 RR027154 awarded by the National Institutes of Health. The government has certain rights in the invention.

### SEQUENCE LISTING

The instant application contains a Sequence Listing which is being submitted in ASCII format via EFS-Web and is hereby incorporated by reference in its entirety. Said ASCII copy, created on Mar. 23, 2022, is named G8118-03101_ST25.txt and is 81,149 bytes in size.

### FIELD OF THE INVENTION

The invention provides methods and compositions for treating or ameliorating Alzheimer's disease. The invention provides agents that reduce Aβ inclusions and plaque accumulation, inhibit dendrite spine loss, and restore neurogenesis.

### BACKGROUND OF THE INVENTION

Alzheimer's disease (AD) is an age-related neurodegenerative disorder characterized by the presence of amyloid β (Aβ) plaques (1,2). Human AD plaques contain a wide variety of different soluble oligomeric Aβ species (3), some of which have been postulated as the toxic species responsible for the pathogenesis and spread of AD (4-8). Inhibiting Aβ production and facilitating its clearance represent promising therapeutic strategies for treating AD. Three proteases control the processing of APP into Aβ: α-secretase cleaves APP between what would be residues 16(Lys) and 17(Leu) of Aβ, releasing a soluble α fragment of APP (sAPPα) and leaving a non-amyloidogenic membrane-bound protein; alternatively, β-secretase can cleave APP to form the amino terminal of Aβ, releasing a slightly shorter soluble β fragment of APP molecule (sAPPP) and leaving a potentially amyloidogenic membrane bound protein; γ-secretase cleaves the membrane-bound fragment at the C-terminal of Aβ to release the amyloidogenic Aβ protein. The j-site APP cleaving enzyme-1 (BACE-1) is the predominant enzyme involved in β-secretase processing of APP and is a therapeutic target for treatment of AD.

The hypothesis that oligomeric Aβ species are responsible for AD pathogenesis rather than the fibrillar amyloid plaques is supported by many studies including a mouse model engineered to express oligomeric Aβ but not plaques (APP^{E693Q})(6,9) where mice engineered to convert oligomers into plaques (APP^{E693Q}/PS1ΔE9) were not impaired to a greater extent than the mice generating only oligomeric Aβ(10).

A variety of different oligomeric Aβ conformations have been detected using atomic force microscopy (AFM) imaging (11-18). AFM can be used to estimate the number of monomeric Aβ units in each oligomeric assembly based on the particle size. For example, particles with an average diameter of ˜10 nanometers corresponded to high-molecular-weight (HMW) oligomers and ones with average diameter 3-6 nanometer corresponded to low-molecular-weight (LMW) oligomers (12,13,15-19). The different aggregate structures could have different consequences as the higher molecular weight Aβ oligomers accelerated Aβ fibrillogenesis whereas other complexes have been shown to create pores that could facilitate disintegration of membrane structures(19).

Since distinct oligomeric Aβ variants can induce toxicity by different mechanisms, treatments that inhibit the toxicity of the specific Aβ species are desired.

Citation or identification of any document in this application is not an admission that such document is available as prior art to the present invention.

### SUMMARY OF THE INVENTION

While a number of different Aβ oligomeric species have been identified, precise identification of the toxic Aβ oligomer structures responsible for neurodegeneration is still lacking. The invention provides compositions and methods for selective targeting of distinct conformational Aβ species with associated therapeutic benefits for treatment or amelioration of Aβ diseases and disorders.

Aβ is formed by sequential cleavage of the amyloid precursor protein (APP). APP can be processed by α-, β- and γ-secretases; Aβ protein is generated by successive action of the β and γ secretases. The γ secretase, which produces the C-terminal end of the Aβ peptide, cleaves within the transmembrane region of APP and can generate a number of isoforms of between 36 and 43 amino acid residues in length. The most common isoforms are Aβ 40 and Aβ 42; the longer form is typically produced by cleavage that occurs in the endoplasmic reticulum, while the shorter form is produced by cleavage in the trans-Golgi network. The Aβ 40 form is the more common of the two, but Aβ 42 is the more fibrillogenic.

In an aspect, the invention provides antibodies and pharmaceutical compositions thereof that bind to Aβ oligomers. Single chain antibody variable domain fragments (scFv) C6T, A4, and E1 bind conformationally distinct toxic oligomeric Aβ species. The A4 scFv binds an oligomeric Aβ variant that can be generated synthetically by incubating monomeric Aβ in a test tube. In contrast, the C6T scFv binds an oligomeric Aβ variant isolated from human brain tissue but does not bind synthetically generated Aβ variants. The oligomeric Aβ species recognized by C6T is considered to be a cell derived oligomeric Aβ species. Since both of these scFvs reduced toxicity of Aβ in in vitro assays, the scFvs are promising therapeutics to selectively target different Aβ variants. C6T- and A4-derived antibodies were constructed to facilitate transport across the blood brain barrier (BBB) reduce or inhibit Aβ toxicity in brains in an animal model of AD. The antibodies can be provided by a vector targeted for expression in cells of a subject or administered directly.

In an aspect, the invention provides antibodies and antibody fragments that bind to toxic Aβ species. In certain embodiments, an antibody is provided which comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence AISGSGGSTYYADSVKG (SEQ ID NO:4); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence KSSQSVLYNSNNKNYLA (SEQ ID NO:8); (e) a light chain complementarity determining region 2 (VL-CDR2) comprising the amino acid sequence WASTRES (SEQ ID NO:10); and (f) a light chain complementarity determining region 3 (VL-CDR3) comprising the amino acid sequence QQFYSTPPT (SEQ ID NO:12). In certain embodiments, the antibody is capable of transport across the blood brain barrier (BBB). In certain embodiments, the antibody is designed for transport across the BBB.

In certain embodiments, the antibody comprises (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence SGSGGS (SEQ ID NO:16); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence SQSVLYNSNNKNY (SEQ ID NO:18); (e) a light chain complementarity determining region 2 (VL-CDR2) comprising the amino acid sequence WAS (SEQ ID NO:20); and (f) a light chain complementarity determining region 3 (VL-CDR3) comprising the amino acid sequence FYSTPP (SEQ ID NO:22). In certain embodiments, the antibody is capable of transport across the blood brain barrier (BBB). In certain embodiments, the antibody is designed for transport across the BBB.

In certain embodiments, the antibody comprises (a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37);

In certain embodiments, the antibody comprises: (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence QHTGAP (SEQ ID NO:39); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence SQSISSY (SEQ ID NO:41); (e) a light chain complementarity determining region 2 (VL-CDR2) comprising the amino acid sequence SAS (SEQ ID NO:43); and (f) a light chain complementarity determining region 3 (VL-CDR3) comprising the amino acid sequence RETGPK (SEQ ID NO:45).

In certain embodiments, the antibody comprises (a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37);

In certain embodiments, the antibody comprises: (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence QHTGAP (SEQ ID NO:39); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence SQSISSY (SEQ ID NO:41); (e) a light chain complementarity determining region 2 (VL-CDR2) comprising the amino acid sequence SAS (SEQ ID NO:43); and (f) a light chain complementarity determining region 3 (VL-CDR3) comprising the amino acid sequence RETGPK (SEQ ID NO:45).

In certain embodiments, the antibody comprises (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence SIQPEGRRTAYVDSVK (SEQ ID NO:50); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence AASSLQS (SEQ ID NO:54); and (f) a light chain complementarity determining region 3 (VL-CDR3) comprising the amino acid sequence QQSYSTPNT (SEQ ID NO:56);

In certain embodiments, the antibody comprises: (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence QPEGRR (SEQ ID NO:58); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence SQSISSY (SEQ ID NO:41); (e) a light chain complementarity determining region 2 (VL-CDR2) comprising the amino acid sequence AAS (SEQ ID NO:60); and (f) a light chain complementarity determining region 3 (VL-CDR3) comprising the amino acid sequence SYSTPN (SEQ ID NO:62).

In a further aspect, the present invention provides a method of treating a disease characterized by Aβ oligomer activity, comprising administering an effective amount of an anti-Aβ oligomer antibody of the present invention. In an embodiment, the present invention provides a method of treating a disease characterized by Aβ oligomer activity, comprising administering to a subject in need thereof a pharmaceutical composition comprising an antibody of the present invention. In certain embodiments, the subject is a human subject.

In certain embodiments, the present invention provides a method of treating, ameliorating, or preventing Aβ diseases or disorders or symptoms thereof, including but not limited to clinical or pre-clinical Alzheimer's disease, Down's syndrome, or clinical or pre-clinical cerebral amyloid angiopathy (CAA), comprising administering to said subject an effective amount of an antibody of the present invention. In certain embodiments, the present invention provides a method of treating, ameliorating, or preventing Aβ diseases or disorders or symptoms thereof, including but not limited to clinical or pre-clinical Alzheimer's disease, Down's syndrome, and clinical or pre-clinical CAA comprising administering to a subject in need thereof an effective amount of a pharmaceutical composition comprising an antibody of the present invention. In certain embodiments, the present invention provides a method of treating, ameliorating, or preventing a condition selected from prodromal AD (sometimes also referred to as Aβ-related mild cognitive impairment, or MCI), mild AD, moderate AD, and severe AD, comprising administering to a patient in need thereof an effective amount of an antibody of the present invention. In certain embodiments, the present invention provides a method of treating, ameliorating, or preventing a condition selected from prodromal AD, mild AD, moderate AD, and severe AD, comprising administering to a patient in need thereof a pharmaceutical composition comprising an antibody of the present invention.

In certain embodiments the present invention provides a method of slowing cognitive decline in a patient diagnosed with an Aβ disease or disorder, including but not limited to clinical or pre-clinical Alzheimer's disease, Down's syndrome, and clinical or pre-clinical cerebral amyloid angiopathy (CAA), comprising administering an antibody of the present invention or a pharmaceutical composition comprising an antibody of the present invention. In certain embodiments, the present invention provides a method of slowing cognitive decline in a patient diagnosed with a condition selected from prodromal AD, mild AD, moderate AD and severe AD, comprising administering a pharmaceutical composition comprising an antibody of the present invention. In another such embodiment the present invention provides a method of slowing cognitive decline in a patient diagnosed with pre-clinical Alzheimer's disease, clinical Alzheimer's disease, Down's syndrome, or clinical or pre-clinical cerebral amyloid angiopathy, comprising administering an effective amount of an antibody of the present invention. More particularly, the present invention further provides a method of slowing cognitive decline in a patient diagnosed with a condition selected from prodromal AD, mild AD, moderate AD and severe AD, comprising administering an effective amount of an antibody of the present invention.

In certain embodiments, the present invention provides a method of reducing brain amyloid plaque load in a subject, including but not limited to a patient diagnosed with pre-clinical or clinical Alzheimer's disease, comprising administering an antibody of the present invention or a pharmaceutical composition comprising an antibody of the present invention. In certain embodiments, the present invention provides a method of reducing brain Aβ amyloid plaque load in a patient diagnosed with a condition selected from prodromal AD, mild AD, moderate AD or severe AD, comprising administering a pharmaceutical composition comprising an antibody of the present invention. Without being bound by theory, brain amyloid plaque load and/or accumulation of brain amyloid plaque is reduced when Aβ oligomer and/or Aβ oligomer activity is reduced.

In certain embodiments the present invention provides a method of preventing memory loss or cognitive decline in an asymptomatic patient comprising administering to the patient a pharmaceutical composition comprising an antibody of the present invention. In a preferred embodiment, the patient has low levels of Aβ in the cerebrospinal fluid (CSF) or Aβ plaques in the brain. In another embodiment the present invention provides a method of treating asymptomatic patients known to have an Alzheimer's disease-causing genetic mutation, comprising administering to the said patient a pharmaceutical composition comprising an antibody of the present invention. In certain embodiments, the present invention provides a method of treating asymptomatic patients known to have a PSEN1 E280A Alzheimer's disease-causing genetic mutation (Paisa mutation), comprising administering to the said patient a pharmaceutical composition comprising an antibody of the present invention. In certain embodiments, the present invention provides a method of treating asymptomatic patients with a genetic mutation, such as a mutation in the APP, PSEN1, or PSEN2 gene, that causes autosomal-dominant Alzheimer's disease, comprising administering to the said patient a pharmaceutical composition comprising an antibody of the present invention.

In certain embodiments, the invention provides method and compositions for treating or ameliorating Aβ diseases and disorders including, without limitation, cerebral amyloid angiopathy (also known as congophilic angiopathy), Lewy body dementia, retinal ganglion cell degeneration (such as in glaucoma), sporadic inclusion body myositis (sIBM) and hereditary inclusion body myopathy (hIBM).

In certain embodiments, the invention provides a method to treat a disease characterized by increased Aβ expression, deposition, aggregation or plaque formation. In certain embodiments, the compositions are used to reduce, prevent or delay Aβ expression, deposition, aggregation or plaque formation in a subject in need thereof. The method typically includes administering to the subject a composition including an effective amount of a composition to reduce, delay, or inhibit the level, formation, or production of Aβ in the subject compared to a control.

In certain embodiments the present invention provides a method of preventing memory loss or cognitive decline in asymptomatic patients known to have an Alzheimer's disease-causing genetic mutation, comprising administering to the said patient a pharmaceutical composition comprising an antibody of the present invention. In a particular embodiment, the present invention provides a method of preventing memory loss or cognitive decline in asymptomatic patients known to have a PSEN1 E280A Alzheimer's disease-causing genetic mutation (Paisa mutation), comprising administering to the said patient a pharmaceutical composition comprising an antibody of the present invention. In certain embodiments, the present invention provides a method of preventing memory loss or cognitive decline in asymptomatic patients with a genetic mutation that causes autosomal-dominant Alzheimer's disease, comprising administering to the said patient a pharmaceutical composition comprising an antibody of the present invention

In certain embodiments the present invention provides a method of slowing cognitive decline in an asymptomatic patient known to have an Alzheimer's disease-causing genetic mutation, comprising administering to the said patient a pharmaceutical composition comprising an antibody of the present invention. In certain embodiments, the present invention provides a method of slowing cognitive decline in asymptomatic patients known to have a PSEN1 E280A Alzheimer's disease-causing genetic mutation (Paisa mutation), comprising administering to the said patient a pharmaceutical composition comprising an antibody of the present invention. In another particular embodiment, the present invention provides a method of slowing cognitive decline in asymptomatic patients with a genetic mutation that causes autosomal-dominant Alzheimer's disease, comprising administering to the said patient a pharmaceutical composition comprising an antibody of the present invention.

In an aspect, the invention provides a method of reducing or inhibiting Aβ toxicity in the brain of a subject, which comprises administering to the subject or expressing in the subject an effective amount of an antibody that selectively binds to oligomeric Aβ and is engineered for transport across the blood brain barrier (BBB).

In certain embodiments, administration of the antibody treats or ameliorates a symptom of Alzeheimer's disease. In certain embodiments, administration of the antibody reduces intracellular Aβ inclusions. In certain embodiments, administration of the antibody reduces or inhibits amyloid accumulation. In certain embodiments, administration of the antibody reduces or inhibits fibrillary deposits. In certain embodiments, administration of the antibody reduces or inhibits microgliosis, astrogliosis, and/or dendritic spine loss. In certain embodiments, administration of the antibody increases neurogenesis. In certain embodiments, administration of the antibody reduces or inhibits decline in cognitive impairment or improves cognition.

The effects of administration are observable and can be confirmed in vitro in a live subject or model, or upon assay of tissue samples. For example, and as disclosed herein, intracellular and extracellular Aβ structures, amyloid plaque accumulation, and fibrillary deposits can be determined using isoform-specific reagents.

In a further aspect, the present invention provides an anti-Aβ oligomer monoclonal antibody or antigen-binding fragment thereof, for use in therapy. In a preferred embodiment, the present invention provides an anti-Aβ oligomer monoclonal antibody or antigen-binding fragment thereof, for use in the treatment of a condition selected from clinical or pre-clinical Alzheimer's disease, prodromal Alzheimer's disease, Down's syndrome, or clinical or pre-clinical CAA. In a more preferred embodiment, the present invention provides an anti-Aβ oligomer monoclonal antibody or antigen-binding fragment thereof, for use in the treatment of Alzheimer's disease. In another preferred embodiment, the present invention provides an anti-Aβ oligomer monoclonal antibody or antigen-binding fragment thereof, for use in the prevention of a condition selected from clinical or pre-clinical Alzheimer's disease, prodromal Alzheimer's disease, clinical or pre-clinical CAA. In a more preferred embodiment, the present invention provides an anti-Aβ oligomer monoclonal antibody or antigen-binding fragment thereof for use in the prevention of Alzheimer's disease.

In an aspect, the invention provides methods and reagents for determining whether a candidate agent is useful for treating or ameliorating Alzheimer's disease. In certain embodiments, the method comprises comparing the candidate agent to a control reagent, wherein the control reagent comprises or expresses an antibody engineered for transport across the blood brain barrier (BBB) which comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence AISGSGGSTYYADSVKG (SEQ ID NO:4); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence KSSQSVLYNSNNKNYLA (SEQ ID NO:8); (e) a light chain complementarity determining region 2 (VL-CDR2) comprising the amino acid sequence WASTRES (SEQ ID NO:10); and (f) a light chain complementarity determining region 3 (VL-CDR3) comprising the amino acid sequence QQFYSTPPT (SEQ ID NO:12).

In certain embodiments, the method comprises comparing the candidate agent to a control reagent, wherein the control reagent comprises or expresses an antibody engineered for transport across the blood brain barrier (BBB) which comprises (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence SGSGGS (SEQ ID NO:16); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence SQSVLYNSNNKNY (SEQ ID NO:18); (e) a light chain complementarity determining region 2 (VL-CDR2) comprising the amino acid sequence WAS (SEQ ID NO:20); and (f) a light chain complementarity determining region 3 (VL-CDR3) comprising the amino acid sequence FYSTPP (SEQ ID NO:22).

In certain embodiments, the method comprises comparing the candidate agent to a control reagent, wherein the control reagent comprises or expresses an antibody engineered for transport across the blood brain barrier (BBB) which comprises (a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37).

In certain embodiments, the method comprises comparing the candidate agent to a control reagent, wherein the control reagent comprises or expresses an antibody engineered for transport across the blood brain barrier (BBB) which comprises (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence QHTGAP (SEQ ID NO:39); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence SQSISSY (SEQ ID NO:41); (e) a light chain complementarity determining region 2 (VL-CDR2) comprising the amino acid sequence SAS (SEQ ID NO:43); and (f) a light chain complementarity determining region 3 (VL-CDR3) comprising the amino acid sequence RETGPK (SEQ ID NO:45).

In certain embodiments, the method comprises comparing the candidate agent to a control reagent, wherein the control reagent comprises or expresses an antibody engineered for transport across the blood brain barrier (BBB) which comprises (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence SIQPEGRRTAYVDSVK (SEQ ID NO:50); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence AASSLQS (SEQ ID NO:54); and (f) a light chain complementarity determining region 3 (VL-CDR3) comprising the amino acid sequence QQSYSTPNT (SEQ ID NO:56);

In certain embodiments, the method comprises comparing the candidate agent to a control reagent, wherein the control reagent comprises or expresses an antibody engineered for transport across the blood brain barrier (BBB) which comprises: (a) a heavy chain complementarity determining region 1 (VH-CDR1) comprising the amino acid sequence GFTFSSY (SEQ ID NO:14); (b) a heavy chain complementarity determining region 2 (VH-CDR2) comprising the amino acid sequence QPEGRR (SEQ ID NO:58); (c) a heavy chain complementarity determining region 3 (VH-CDR3) comprising the amino acid sequence PPERFDY (SEQ ID NO:52; (d) a light chain complementarity determining region 1 (VL-CDR1) comprising the amino acid sequence SQSISSY (SEQ ID NO:41); (e) a light chain complementarity determining region 2 (VL-CDR2) comprising the amino acid sequence AAS (SEQ ID NO:60); and (f) a light chain complementarity determining region 3 (VL-CDR3) comprising the amino acid sequence SYSTPN (SEQ ID NO:62).

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The following detailed description, given by way of example, but not intended to limit the invention solely to the specific embodiments described, may best be understood in conjunction with the accompanying drawings.

FIG. 1. A4 and C6T immunopositive structures in post-mortem human brain tissue. Aβ aggregates were stained using an antibody against Aβ1-17 (clone 6E10, red). Little staining of A4 and C6T, as well as 6E10 was observed in the human brains with non-dementia (ND). Counter staining by DAPI. A) The oligomeric Aβ variants recognized by A4 scFv (green), together with Aβ aggregates stained by antibody 6E10 (red, arrowhead), in AD brain tissue. B) The oligomeric Aβ variants recognized by C6T scFv (green), together with Aβ plaques shown by 6E10 (red, arrowhead), as well as C6T-positive staining around the nuclei (arrow), in AD brain tissue. C) The extracellular and intracellular aggregates that C6T recognized were observed (green) in AD but not ND brain tissue. A control using secondary antibody against cMyc without the primary scFv is also shown. The neuronal cells were stained by specific antibody against MAP2 (red, arrow). Bars: 20 μm.

FIG. 2. Localization and levels of A4 and C6T recognized Aβ structures in brain tissue from APP/PS1 mice. Aβ accumulation was stained by a specific antibody against A31-17 (6E10, red). Counter staining by DAPI. A) Staining with purified A4 scFv in the APP/PS1 mice brain tissue. B) The staining of C6T-recognized oligomeric Aβ (green) and Aβ aggregates (6E10, red) in WT-GFP, Tg-GFP, Tg-A4 and Tg-C6T mouse brain tissue and negative staining of A4 and C6T, as well as 6E10 in the brains of WT mice. Bars: 50 μm. C) Levels of oligomeric Aβ reactive with A4 scFv following treatment with rAAV-GFP, rAAV-A4 and rAAV-C6T. D) Levels of oligomeric Aβ reactive with C6T scFv following treatment with rAAV-GFP, rAAV-A4 and rAAV-C6T. All data were expressed as fold increase relative to WT-GFP mice in the cortex and hippocampus. The quantitative assay was performed with the mice of WT-GFP (n=3), Tg-GFP (n=10), Tg-A4 (n=7) and Tg-C6T (n=9). ^{###}p<0.001 to age-matched WT-GFP mice. *p<0.05 and ***p<0.001 to littermate Tg-GFP vehicle mice, and ^{&}p<0.05 to littermate Tg-A4 mice.

FIG. 3. Cellular location of scFv-recognized oligomeric variants. HEK293 cells and HEK293 cells expressing the human APPswe mutation were incubated in DMEM with 0.5% FBS for 24 h and fixed with 4% PFA for 10 min. A) Oligomeric Aβ species recognized by A4 were immunolabeled with an antibody against the c-Myc tag (green) on the scFv, and by a monoclonal antibody 6E10 (red) against Aβ. B) Oligomeric Aβ species recognized by C6T were immunolabeled with an antibody against c-Myc (green), and Aβ by monoclonal antibody 6E10 (red). Nuclei were counter stained with DAPI (blue). Bar: 20 μm.

FIG. 4. Aβ staining in APP/PS1 mouse brain tissue. A) Aβ deposits were stained using anti-Aβ antibody 6E10 staining in the cortex (top) and the hippocampus (bottom) following the treatment with rAAV-GFP, rAAV-A4 and rAAV-C6T. B) and C) The number of 6E10-positive deposits was counted and averaged per section in the cortex (B) and the hippocampus (C) of mice treated with rAAV-GFP, rAAV-A4 and rAAV-C6T. D) Fibrillar deposits were verified by Congo-red staining in the cortex (top) and the hippocampus (bottom). E) and F) The number of fibrillar deposits was counted and averaged per section in the cortex (E) and the hippocampus (F). Nuclei were counterstained with haemotaxylin. WT-GFP (n=3), Tg-GFP (n=15), Tg-A4 (n=7) and Tg-C6T (n=9). *p<0.05 and **p<0.01 to Tg-GFP mice and ^{&}p<0.05 to littermate Tg-A4 mice.

FIG. 5. Gliosis in response to Aβ in APP/PS1 mouse brain tissue. A) The immune cell microglia were immunostained by specific antibody against Iba1 (green). Aβ deposits were visualized by antibody against Aβ1-17 (clone: 6E10, red). Counter stain by DAPI (blue). B) Positive staining of microglia was expressed as a percentage of total area. C. Reactive astrocytes were visualized using an antibody against GFAP. The fibrillar deposits were visualized by Congo-Red staining. Cell nuclei were counter stained by haemotaxylin. D) Positive staining of reactive astrocytes was expressed as a percentage of total area. WT-GFP (n=3), Tg-GFP (n=5), Tg-A4 (n=5) and Tg-C6T (n=5). ^{###}p<0.001 to age-matched WT-GFP mice, *p<0.05 and ***p<0.001 to littermate Tg-GFP group, ^{&}p<0.05 to littermate Tg-A4 group.

FIG. 6. Changes in dendritic spines and synapses in APP/PS1 mouse brain tissue. A) Neuronal dendrites were immunostained using an antibody against MAP2 (green) in the cortex. B) The dendritic area of MAP2-positive staining was expressed as a percentage of total captured cortex regions. C) Neuronal dendrites were visualized using an antibody against MAP2 (green) in CA3 region of the hippocampus. Aberrant dendritic arrangements are indicated (arrow). D) The dendritic area of MAP2-positive was expressed as a percentage of total captured hippocampal regions. E) The synapses were labeled with a specific antibody against synaptophysin (SYP, green). F) The area of SYP-positive staining was expressed as a percentage of total captured regions. In the images, Aβ deposits were labeled by antibody 6E10 (red). Counter staining by DAPI (blue). WT-GFP (n=3), Tg-GFP (n=5) Tg-A4 (n=5), and Tg-C6T (n=5). Statistical analysis was valued as ^{###}p<0.001 to age-matched WT-GFP mice, ***p<0.001 to littermate Tg-GFP vehicle group, ^{&&}p<0.001 and ^{&&&} p<0.001 to littermate Tg-A4 group.

FIG. 7. Changes in hippocampal neurogenesis and survival rate. A) The newborn immature neurons in the subgranular zone of hippocampus were visualized with an antibody against DCX (red). Cell nuclei were counter stained by DAPI (blue). B) The number of DCX-positive cells was counted and averaged per sections. ^{##}p<0.01 to age-matched WT-GFP mice, *p<0.05 and ***p<0.001 to littermate Tg-GFP vehicle group, ^{&&&} p<0.001 to Tg-A4 mice. C) The survival rate of the APP/PS1 mice receiving rAAV-GFP, rAAV-C6T and rAAV-A4 with time, compared to that of age-matched WT-GFP mice as controls. At age of two months (the time point of viral injection), the numbers of mice injected were: WT-GFP (n=15), Tg-GFP (n=20), Tg-A4 (n=12) and Tg-C6T (n=10). Seven months later, the number of mice that survived were 15, 12, 7 and 9, respectively.

FIG. 8. Viral infection in brains of mice receiving rAAV. A) Active rAAVs were stained with an antibody against rAAV capsid AAV2 (red) in the hepatic cells of the mice administered rAAV intraperitoneally. B) The rAAV capsid AAV2 was immunostained (red) in brain sections of the mice receiving rAAV. C) The green fluorescence of GFP expression was observed in the liver tissues of mice injected with rAAV-GFP, rAAV-A4 and rAAV-C6T. D) No protein expression of GFP (green) was observed in the brain sections of mice injected with rAAV-GFP, rAAV-A4 and rAAV-C6T. Cell nuclei were counterstained with DAPI (blue).

FIG. 9. Delivery of scFv to brains receiving rAAV. A) FLAG immunostaining (red) in the hepatic tissues of the mice receiving rAAV-GFP, rAAV-A4 or rAAV-C6T. B) FLAG immunostaining (red) in the brain tissues of the mice receiving rAAV-GFP, rAAV-A4 or rAAV-C6T. C) ELISA measured scFv concentration by rabbit anti-tag FLAG antibody. Data were expressed as pg/mg of the brain tissue receiving the application of rAAV-GFP, rAAV-A4 or rAAV-C6T. n=5 in each group.

FIG. 10. APP amyloidogenic processing in the brains of APP/PS1 mice. A) Representative expression showing the expression of BACE1 as well as sAPPβ-sw in the cortex by Western blot. B) Normalized to age-matched Tg-GFP, semi-quantification showed the total amount of BACE1 and sAPPβ-sw in the cortex among the mice administering WT-GFP, Tg-GFP, Tg-A4 and Tg-C6T. n=5 each group, p >0.05. C) Representative image showing the expression of human APP and its C-terminal fragments, CTFα, and CTFβ, in the cortex by Western blot. D) Normalized to age-matched Tg-GFP, semi-quantification showed the amount of APP and CTFs in the cortex among the mice administering Tg-GFP, Tg-A4 and Tg-C6T. n=5 each group, p >0.05.

FIG. 11. IHC staining of human post-mortem brain tissue. Braak Stage 0 (38 years old ApoE3/3), Stage II (79 years old, ApoE3/3), Stage VI (88 years old, ApoE4/4). A) Braak stage 0 case has extensive ubiquitin staining that completely overlaps with C6T staining indicating a functional UPS. B, D): Braak II case has some neurons with abundant ubiquitin staining (B) or limited P62 staining (D) indicating a functional UPS, and some neurons with significant C6T staining and little ubiquitin (B) or extensive P62 staining (D) indicating a disrupted UPS. C, E) Braak stage VI case has C6T staining with little cytoplasmic but some axonal ubiquitin staining (C) and minimal P62 staining (E) indicating a dysfunctional UPS.

FIG. 12. IHC staining of APP/PS1 mouse brain tissue treated with sham (GFP) or scFv targeting extracellular Aβ (A4) or intracellular Aβ (C6T). Full arrow indicates neurons with high levels of ubiquitin and low levels of C6T. Arrowhead indicate neurons with high levels of C6T, but low ubiquitin. Treatment with C6T essentially restores UPS pathway to wild type phenotype while treatment with A4 is similar to sham treatment.

### DETAILED DESCRIPTION OF THE INVENTION

The invention provides antibodies and antibody fragments the bind to toxic Aβ species. In certain embodiments, an antibody engineered for transport across the blood brain barrier (BBB) is provided.

Binding specificities of antibodies including antigen binding fragments of the invention are determined primarily by CDRs as set forth herein. Antibodies C6T, A4 and E1 selectively bind to conformational variants of oligomeric Aβ (Table 1). Differences in the oligomeric Aβ conformational variants may reside in aggregation of the oligomers and the enritonment in which they are produced, e.g., intracellularly or extracellularly. Aspects of the C6T, A4, and E1 antibodies are further described in the inventors' publications, for example, international PCT patent publication WO 2013/148166. While there may be benefits associated with the smaller size of scFvs, Fabs, and nanobodies, the invention includes antibodies of any kind as well as antigen binding fragments thereof, all of which can be engineered to cross the BBB.

Without being bound by theory, the antibodies of the invention bind to and reduce the amount and/or toxicity of Aβ oligomers. The benefit may result from internalization of the antibodies by neurons, from binding of the antibodies to antigen that is readily shuttled into and out of neurons, from both, or from another mechanism.

As used herein, the term “antibody” includes scFv, humanized, fully human or chimeric antibodies, single-chain antibodies, diabodies, and antigen-binding fragments of antibodies that do not contain the Fc region (e.g., Fab fragments). In certain embodiments, the antibody is a human antibody or a humanized antibody. A “humanized” antibody contains only the three CDRs (complementarity determining regions) and sometimes a few carefully selected “framework” residues (the non-CDR portions of the variable regions) from each donor antibody variable region recombinantly linked onto the corresponding frameworks and constant regions of a human antibody sequence. A “fully humanized antibody” is created in a hybridoma from mice genetically engineered to have only human-derived antibody genes or by selection from a phage-display library of human-derived antibody genes.

As used herein, the term “antibody” includes a single-chain variable fragment (scFv or “nanobody”), humanized, fully human or chimeric antibodies, single-chain antibodies, diabodies, and antigen-binding fragments of antibodies (e.g., Fab fragments). A scFv is a fusion protein of the variable region of the heavy (V_{H}) and light chains (V_{L}) of an immunoglobulin that is connected by means of a linker peptide. The linker is usually short, about 10-25 amino acids in length. If flexibility is important, the linker will contain a significant number of glycines. If solubility is important, serines or threonines will be utilized in the linker. The linker may link the amino-terminus of the V_{H} to the carboxy-terminus of the V_{L}, or the linker may link the carboxy-terminus of the V_{H} to the amino-terminus of the V_{L}. Divalent (also called bivalent) scFvs can be generated by linking two scFvs. For example, a divalent scFv can be made by generating a single peptide containing two V_{H} and two V_{L} regions. Alternatively, two peptides, each containing a single V_{H} and a single V_{L} region can be dimerized (also called “diabodies”). Holliger et al., “Diabodies: small bivalent and bispecific antibody fragments,” PNAS, July 1993, 90:6444-6448. Bivalency allows antibodies to bind to multimeric antigens with high avidity, and bispecificity allows the cross-linking of two antigens.

As used herein, the term “monoclonal antibody” refers to an antibody obtained from a group of substantially homogeneous antibodies, that is, an antibody group wherein the antibodies constituting the group are homogeneous except for naturally occurring mutants that exist in a small amount. Monoclonal antibodies are highly specific and interact with a single antigenic site. Furthermore, each monoclonal antibody targets a single antigenic determinant (epitope) on an antigen, as compared to common polyclonal antibody preparations that typically contain various antibodies against diverse antigenic determinants. In addition to their specificity, monoclonal antibodies are advantageous in that they are produced from hybridoma cultures not contaminated with other immunoglobulins.

The adjective “monoclonal” indicates a characteristic of antibodies obtained from a substantially homogeneous group of antibodies, and does not specify antibodies produced by a particular method. For example, a monoclonal antibody to be used in the present invention can be produced by, for example, hybridoma methods (Kohler and Milstein, Nature 256:495, 1975) or recombination methods (U.S. Pat. No. 4,816,567). The monoclonal antibodies used in the present invention can be also isolated from a phage antibody library (Clackson et al., Nature 352:624-628, 1991; Marks et al., J. Mol. Biol. 222:581-597, 1991). The monoclonal antibodies of the present invention particularly comprise “chimeric” antibodies (immunoglobulins), wherein a part of a heavy (H) chain and/or light (L) chain is derived from a specific species or a specific antibody class or subclass, and the remaining portion of the chain is derived from another species, or another antibody class or subclass. Furthermore, mutant antibodies and antibody fragments thereof are also comprised in the present invention (U.S. Pat. No. 4,816,567; Morrison et al., Proc. Natl. Acad. Sci. USA 81:6851-6855, 1984).

As used herein, the term “mutant antibody” refers to an antibody comprising a variant amino acid sequence in which one or more amino acid residues have been altered. For example, the variable region of an antibody can be modified to improve its biological properties, such as antigen binding. Such modifications can be achieved by site-directed mutagenesis (see Kunkel, Proc. Natl. Acad. Sci. USA 82: 488 (1985)), PCR-based mutagenesis, cassette mutagenesis, and the like. Such mutants comprise an amino acid sequence which is at least 70% identical to the amino acid sequence of a heavy or light chain variable region of the antibody, more preferably at least 75%, even more preferably at least 80%, still more preferably at least 85%, yet more preferably at least 90%, and most preferably at least 95% identical. As used herein, the term “sequence identity” is defined as the percentage of residues identical to those in the antibody's original amino acid sequence, determined after the sequences are aligned and gaps are appropriately introduced to maximize the sequence identity as necessary.

Specifically, the identity of one nucleotide sequence or amino acid sequence to another can be determined using the algorithm BLAST, by Karlin and Altschul (Proc. Natl. Acad. Sci. USA, 90: 5873-5877, 1993). Programs such as BLASTN and BLASTX were developed based on this algorithm (Altschul et al., J. Mol. Biol. 215: 403-410, 1990). To analyze nucleotide sequences according to BLASTN based on BLAST, the parameters are set, for example, as score=100 and wordlength=12. On the other hand, parameters used for the analysis of amino acid sequences by BLASTX based on BLAST include, for example, score=50 and wordlength=3. Default parameters for each program are used when using the BLAST and Gapped BLAST programs. Specific techniques for such analyses are known in the art (see the website of the National Center for Biotechnology Information (NCBI), Basic Local Alignment Search Tool (BLAST); (ncbi.nlm.nih.gov).

In another embodiment, antibodies or antibody fragments can be isolated from an antibody phage library, produced by using the technique reported by McCafferty et al. (Nature 348:552-554 (1990)). Clackson et al. (Nature 352:624-628 (1991)) and Marks et al. (J. Mol. Biol. 222:581-597 (1991)) reported on the respective isolation of mouse and human antibodies from phage libraries. There are also reports that describe the production of high affinity (nM range) human antibodies based on chain shuffling (Marks et al., Bio/Technology 10:779-783 (1992)), and combinatorial infection and in vivo recombination, which are methods for constructing large-scale phage libraries (Waterhouse et al., Nucleic Acids Res. 21:2265-2266 (1993)). These technologies can also be used to isolate monoclonal antibodies, instead of using conventional hybridoma technology for monoclonal antibody production.

Antibodies to be used in the present invention can be purified by a method appropriately selected from known methods, such as the protein A-Sepharose method, hydroxyapatite chromatography, salting-out method with sulfate, ion exchange chromatography, and affinity chromatography, or by the combined use of the same.

The present invention may use recombinant antibodies, produced by gene engineering. The genes encoding the antibodies obtained by a method described above are isolated from the hybridomas. The genes are inserted into an appropriate vector, and then introduced into a host (see, e.g., Carl, A. K. Borrebaeck, James, W. Larrick, Therapeutic Monoclonal Antibodies, Published in the United Kingdom by Macmillan Publishers Ltd, 1990). The present invention provides the nucleic acids encoding the antibodies of the present invention, and vectors comprising these nucleic acids. Specifically, using a reverse transcriptase, cDNAs encoding the variable regions (V regions) of the antibodies are synthesized from the mRNAs of hybridomas. After obtaining the DNAs encoding the variable regions of antibodies of interest, they are ligated with DNAs encoding desired constant regions (C regions) of the antibodies, and the resulting DNA constructs are inserted into expression vectors. Alternatively, the DNAs encoding the variable regions of the antibodies may be inserted into expression vectors comprising the DNAs of the antibody C regions. These are inserted into expression vectors so that the genes are expressed under the regulation of an expression regulatory region, for example, an enhancer and promoter. Then, host cells are transformed with the expression vectors to express the antibodies. The present invention provides cells expressing antibodies of the present invention. The cells expressing antibodies of the present invention include cells and hybridomas transformed with a gene of such an antibody.

The antibodies of the present invention also include antibodies which comprise complementarity-determining regions (CDRs), or regions functionally equivalent to CDRs. The term “functionally equivalent” refers to comprising amino acid sequences similar to the amino acid sequences of CDRs of any of the monoclonal antibodies isolated in the Examples. The term “CDR” refers to a region in an antibody variable region (also called “V region”), and determines the specificity of antigen binding. The H chain and L chain each have three CDRs, designated from the N terminus as CDR1, CDR2, and CDR3. There are four regions flanking these CDRs: these regions are referred to as “framework,” and their amino acid sequences are highly conserved. The CDRs can be transplanted into other antibodies, and thus a recombinant antibody can be prepared by combining CDRs with the framework of a desired antibody. One or more amino acids of a CDR can be modified without losing the ability to bind to its antigen. For example, one or more amino acids in a CDR can be substituted, deleted, and/or added.

Antibody residues that have a substantial impact on affinity and specificity of binding to target antigen are primarily located in CDRs. Kabat et al. compiled and aligned immunoglobulin heavy and light chain sequences and were the first to propose a standardized numbering scheme for the variable regions of immunoglobulins identifying conserved and hypervariable regions and residues. (Kabat E A et al., 1979, Sequences of Immunoglobulin Chains: Tabulation and Analysis of Amino Acid Sequences of Precursors, V-regions, C-regions, J-Chain and BP-Microglobulins, Department of Health, Education, and Welfare, Public Health Service, National Institutes of Health). While the Kabat system is a widely adopted standard for numbering antibody residues, the hypervariable regions defined by Kabat do not exactly match with the structural aspects of antigen-binding loops. Chothia and Lesk developed a structure-based numbering scheme by aligning crystal structures of antibody variable regions and classified CDR loops in a small number of “canonical” classes (Chothia C, et al., 1987, Canonical structures for the hypervariable regions of immunoglobulins. J. Mol. Biol. 196:901-17. doi: 10.1016/0022-2836(87)90412-8). An advantage of the Chothia numbering scheme is that topologically aligned residues from different antibodies are localized at the same position number and the Chothia CDR definition corresponds in most antibody sequences to the structural antigen-binding loop. Lefranc introduced a new system based on germ-line sequences intended to standardize numbering for all proteins of the immunoglobulin superfamily, including T cell receptor chains. (Giudicelli V et al., 1997, IMGT, the international ImMunoGeneTics database. Nucleic Acids Res. 25:206-11), which was then extended to entire variable domains (Lefranc M-P et al., 2003, IMGT unique numbering for immunoglobulin and T cell receptor variable domains and Ig superfamily V-like domains. Dev Comp Immunol. 27:55-77. doi: 10.1016/S0145-305X(02)00039-3). Additional numbering systems have been proposed to align unconventional frameworks (Abhinandan K R et al., 2008, Analysis and improvements to Kabat and structurally correct numbering of antibody variable domains. Mol Immunol. 45:3832-9. doi: 10.1016/j.molimm.2008.05.022) and to subdivide variable chain sequences into multiple fragments including structurally invariant “cores” (Gelfand et al., 1998, Algorithmic determination of core positions in the VL and VH domains of immunoglobulin molecules. J Comput Biol. (1998) 5:467-77). In certain embodiments of the invention, CDRs are defined according to such a standard system as set forth above. In certain embodiments, antibodies of the invention comprise Kabat CDRs of the antibody sequences set forth herein. In certain embodiments, antibodies of the invention comprise Chothia CDRs of the antibody sequences set forth herein. In certain embodiments, antibodies of the invention are identified by all or a subset of IMGT CDR residues of the antibody sequences set forth herein. In certain embodiments, antibodies of the invention comprise specific CDR sequences as specifically or uniquely set forth herein. In certain embodiments, antibodies of the invention are identified by CDR residues defined by two or more systems, comprising e.g., but not limited to, all or a subset of residues of VH-CDR1 according to Kabat, all or a subset of residues of VH-CDR2 according to Chothia, all or a subset of residues of VH-CDR3 according to Kabat, all or a subset of residues of VL-CDR1 according to Kabat, all or a subset of residues of VL-CDR2 according to IMGT, and all or a subset of residues of VL-CDR3 according to Chothia. Table 1 shows the location of IMGT, Kabat, and Chothia CDR by the amino acid numbering of C6.

TABLE 1
CDR amino acids

| | VH-FR1 | VH-CDR1 | VH-FR2 | VH-CDR2 | VH-FR3 | VH-CDR3 | VH-FR4 |
|---|---|---|---|---|---|---|---|
| IMGT | 1-25 | 26-33 | 34-50 | 51-58 | 59-95 | 96-110 | 111-121 |
| Kabat | 1-30 | 31-35 | 36-49 | 50-66 | 67-97 | 98-110 | 111-121 |
| Chothia | 1-25 | 26-32 | 33-51 | 52-57 | 58-97 | 98-110 | 111-121 |
| IMGT | 1-26 | 27-38 | 39-55 | 56-58 | 59-94 | 95-103 | 104-113 |
| Kabat | 1-23 | 24-40 | 41-55 | 56-63 | 64-94 | 95-103 | 104-113 |
| Chothia | 1-25 | 26-38 | 39-55 | 56-59 | 60-96 | 97-102 | 103-113 |

In certain embodiments, an amino acid residue is mutated into one that allows the properties of the amino acid side-chain to be conserved. Examples of the properties of amino acid side chains comprise: hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)).

For a discussion of naturally conserved networks of amino acids that support antibody V(H) and V(L) function, see, e.g., Wang et al., Conserved amino acid networks involved in antibody variable domain interactions. Proteins 2009 July; 76(1):99-114. Wang identifies conserved and non-conserved amino acid pairs in antibody V_{H} and V_{L} domains, the V_{H}-C_{H}1 variable-constant domain interface, as well as in camelid V_{HH} domains, which have evolved to lack interactions with V_{L} and C_{H}1. In certain embodiments, mutations are introduces to optimize biopharmaceutical and biophysical properties, such as efficacy, safety, and manufacturability, and stability of therapeutic antibodies. See, e.g. Douillard et al., Optimization of an Antibody Light Chain Framework Enhances Expression, Biophysical Properties and Pharmacokinetics. Antibodies (Basel) 2019 Sep. 6; 8(3):46.

The number of mutated amino acids is not limited, but in general, the number falls within 40% of amino acids of each CDR, and preferably within 35%, and still more preferably within 30%, or within 25%, or within 20%, or within 15%, or within 10%, or within 5%. In various embodiments, sequences of the CDRs of an antibody of the invention are at least 80%, at least 85%, at least 90%, or at least 95%, or at least 97% identical, or identical to a set of CDRs set forth herein. In certain embodiments, there can be one, two, or three conservative substitutions made in the CDRs set forth herein. The identity of amino acid sequences can be determined as described herein.

In certain embodiments, VH and VL domains comprise frameworks, individually or taken together, at least 70%, at least 75%, at least 80%, at least 85%, at least 90%, at least 95%, at least 96%, at least 97%, at least 98%, at least 99% identical, or identical to the frameworks of antibody variable domains set forth herein. In certain embodiments, VH and VL domains comprise CDRs as set forth above, such as CDRs that are at least 80%, at least 85%, at least 90%, or at least 95%, or at least 97% identical, or identical to a set of CDRs set forth herein and/or comprise one, two, or three conservative substitutions and the variable domain overall is at least 70%, at least 75%, at least 80%, at least 85%, at least 90%, at least 95%, at least 96%, at least 97%, at least 98%, at least 99% identical, or identical to a variable domain set forth herein

In the present invention, recombinant antibodies artificially modified to reduce heterologous antigenicity against humans can be used. Examples include chimeric antibodies and humanized antibodies. These modified antibodies can be produced using known methods. A chimeric antibody includes an antibody comprising variable and constant regions of species that are different to each other, for example, an antibody comprising the antibody heavy chain and light chain variable regions of a nonhuman mammal such as a mouse, and the antibody heavy chain and light chain constant regions of a human. Such an antibody can be obtained by (1) ligating a DNA encoding a variable region of a mouse antibody to a DNA encoding a constant region of a human antibody; (2) incorporating this into an expression vector; and (3) introducing the vector into a host for production of the antibody.

A humanized antibody, which is also called a reshaped human antibody, is obtained by substituting an H or L chain complementarity determining region (CDR) of an antibody of a nonhuman mammal such as a mouse, with the CDR of a human antibody. Conventional genetic recombination techniques for the preparation of such antibodies are known (see, for example, Jones et al., Nature 321: 522-525 (1986); Reichmann et al., Nature 332: 323-329 (1988); Presta Curr. Op. Struct. Biol. 2: 593-596 (1992)). Specifically, a DNA sequence designed to ligate a CDR of a mouse antibody with the framework regions (FRs) of a human antibody is synthesized by PCR, using several oligonucleotides constructed to comprise overlapping portions at their ends. A humanized antibody can be obtained by (1) ligating the resulting DNA to a DNA that encodes a human antibody constant region; (2) incorporating this into an expression vector; and (3) transfecting the vector into a host to produce the antibody (see, European Patent Application No. EP 239,400, and International Patent Application No. WO 96/02576). Human antibody FRs that are ligated via the CDR are selected where the CDR forms a favorable antigen-binding site. The humanized antibody may comprise additional amino acid residue(s) that are not included in the CDRs introduced into the recipient antibody, nor in the framework sequences. Such amino acid residues are usually introduced to more accurately optimize the antibody's ability to recognize and bind to an antigen. For example, as necessary, amino acids in the framework region of an antibody variable region may be substituted such that the CDR of a reshaped human antibody forms an appropriate antigen-binding site (Sato, K. et al., Cancer Res. (1993) 53, 851-856).

The isotypes of the antibodies of the present invention are not limited. The isotypes include, for example, IgG (IgG1, IgG2, IgG3, and IgG4), IgM, IgA (IgA1 and IgA2), IgD, and IgE. The antibodies of the present invention may also be antibody fragments comprising a portion responsible for antigen binding, or a modified fragment thereof. The term “antibody fragment” refers to a portion of a full-length antibody, and generally to a fragment comprising an antigen-binding domain or a variable region. Such antibody fragments include, for example, Fab, F(ab′)₂, Fv, single-chain Fv (scFv) which comprises a heavy chain Fv and a light chain Fv coupled together with an appropriate linker, diabody (diabodies), linear antibodies, and multispecific antibodies prepared from antibody fragments. Additional proteins encompassed by the invention include single domain antibodies, also known as domain antibodies, camelid antibodies and antibodies from cartilaginous fish (i.e., immunoglobulin new antigen receptors (IgNARs)). Generally, camelid antibodies and IgNARs comprise a V_{H}, however lack a V_{L} and are often referred to as heavy chain immunoglobulins. Other “immunoglobulins” include T cell receptors. Previously, antibody fragments were produced by digesting natural antibodies with a protease; currently, methods for expressing them as recombinant antibodies using genetic engineering techniques are also known (see Morimoto et al., Journal of Biochemical and Biophysical Methods 24:107-117 (1992); Brennan et al., Science 229:81 (1985); Co, M. S. et al., J. Immunol., 1994, 152, 2968-2976; Better, M. & Horwitz, A. H., Methods in Enzymology, 1989, 178, 476-496, Academic Press, Inc.; Plueckthun, A. & Skerra, A., Methods in Enzymology, 1989, 178, 476-496, Academic Press, Inc.; Lamoyi, E., Methods in Enzymology, 1989, 121, 663-669; Bird, R. E. et al., TIBTECH, 1991, 9, 132-137).

An “Fv” fragment is the smallest antibody fragment, and contains a complete antigen recognition site and a binding site. This region is a dimer (V_{H}-V_{L} dimer) wherein the variable regions of each of the heavy chain and light chain are strongly connected by a noncovalent bond. The three CDRs of each of the variable regions interact with each other to form an antigen-binding site on the surface of the V_{H}-V_{L} dimer. In other words, a total of six CDRs from the heavy and light chains function together as an antibody's antigen-binding site. However, a variable region (or a half Fv, which contains only three antigen-specific CDRS) alone is also known to be able to recognize and bind to an antigen, although its affinity is lower than the affinity of the entire binding site. Thus, a preferred antibody fragment of the present invention is an Fv fragment, but is not limited thereto. Such an antibody fragment may be a polypeptide which comprises an antibody fragment of heavy or light chain CDRs which are conserved, and which can recognize and bind its antigen.

A Fab fragment (also referred to as F(ab)) also contains a light chain constant region and heavy chain constant region (CH1). For example, papain digestion of an antibody produces the two kinds of fragments: an antigen-binding fragment, called a Fab fragment, containing the variable regions of a heavy chain and light chain, which serve as a single antigen-binding domain; and the remaining portion, which is called an “Fc” because it is readily crystallized. A Fab′ fragment is different from a Fab fragment in that a Fab′ fragment also has several residues derived from the carboxyl terminus of a heavy chain CH1 region, which contains one or more cysteine residues from the hinge region of an antibody. A Fab′ fragment is, however, structurally equivalent to Fab in that both are antigen-binding fragments which comprise the variable regions of a heavy chain and light chain, which serve as a single antigen-binding domain. Herein, an antigen-binding fragment comprising the variable regions of a heavy chain and light chain which serve as a single antigen-binding domain, and which is equivalent to that obtained by papain digestion, is referred to as a “Fab-like antibody,” even when it is not identical to an antibody fragment produced by protease digestion. Fab′-SH is Fab′ with one or more cysteine residues having free thiol groups in its constant region. A F(ab′) fragment is produced by cleaving the disulfide bond between the cysteine residues in the hinge region of F(ab′)₂. Other chemically crosslinked antibody fragments are also known to those skilled in the art. Pepsin digestion of an antibody yields two fragments; one is a F(ab′)₂ fragment which comprises two antigen-binding domains and can cross-react with antigens, and the other is the remaining fragment (referred to as pFc′). Herein, an antibody fragment equivalent to that obtained by pepsin digestion is referred to as a “F(ab′)₂-like antibody” when it comprises two antigen-binding domains and can cross-react with antigens. Such antibody fragments can also be produced, for example, by genetic engineering. Such antibody fragments can also be isolated, for example, from the antibody phage library described above. Alternatively, F(ab′)₂-SH fragments can be recovered directly from hosts, such as E. coli, and then allowed to form F(ab′)₂ fragments by chemical crosslinking (Carter et al., Bio/Technology 10:163-167 (1992)). In an alternative method, F(ab′)2 fragments can be isolated directly from a culture of recombinant hosts.

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (V_{L}) and a heavy chain variable region (V_{H}) in an identical chain are connected via a short linker, for example, a linker of about five residues, so that they cannot bind together. Because the linker between the two is too short, the V_{L} and V_{H} in the same polypeptide chain cannot form a single chain V region fragment, but instead form a dimer. Thus, a diabody has two antigen-binding domains. When the V_{L} and V_{H} regions against the two types of antigens (a and b) are combined to form V_{La}-V_{Hb} and V_{Lb}-V_{Ha} via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, preferably, a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

### Blood Brain Barrier (BBB) and Receptor-Mediated Transcytosis (RMT)

Large molecules gain access to the brain by receptor-mediated transport across the BBB via peptide-specific receptors. Examples include the BBB insulin receptor which mediates transport of insulin from blood to the brain and the BBB transferrin receptor which mediates influx of holo-transferrin. The leptin receptor (LEPR) is expressed at the human BBB and mediates the BBB transport of circulating leptin and the low-density lipoprotein (LDL) LDLR is expressed at the BBB and mediates transport of low-density lipoprotein. See Pardridge, 2017, Delivery of biologics across the blood-brain barrier with molecular trojan horse technology. BioDrugs 31, 503-519. doi: 10.1007/s40259-017-0248-z. The LDLR-binding domain of ApoB is useful to shuttle proteins across the BBB. The LDLR-binding polypeptide SSVIDALQYKLEGTTRLTRKRGLKLATALSLSNKFVEGS (SEQ ID NO:66) is exemplified herein and is non-limiting. LDLR-binding peptides continue to be developed, e.g., HPWCCGLRLDLR (SEQ ID NO:67) and YHFNGCEDPLCR (SEQ ID NO:68) (Andre et al., 2020, Development of an LDL receptor-targeted peptide susceptible to facilitate the brain access of diagnostic or therapeutic agents. Biology 9:161, doi.org/10.3390/biology9070161). David et al. identified a family of peptides that target human LDLR (hLDLR) having small sized and absence of competition with binding of LDL to LDLR. (David et al., 2018, Identification and characterization of highly versatile peptide-vectors that bind non-competitively to the low-density lipoprotein receptor for in vivo targeting and delivery of small molecules and protein cargos. PLoS One 13(2):e0191052).

Cell penetrating peptides (CPPs) may be used to improve delivery across the BBB. A non-limiting example is penetratin (RQIKIWFQNRRMKWKK) (SEQ ID NO:29), a 16 amino-acid polypeptide from the homeodomain of the drosophila Antennapedia protein. (Dupont et al., 2007, Identification of a signal peptide for unconventional secretion. J. Biol. Chem. 282(12):8994). The peptide QSLAQELGLNERQIKIWFQNRRMKWKK (SEQ ID NO:71) can be used to transport molecules across the blood-brain barrier and, more generally, across epithelia forming tight barriers between two compartments. The peptide comprises a sequence containing the nuclear export sequence of the Engrailed protein, and a penetratin sequence from the homeodomain of the Drosophila Antennapedia protein. (Dupont et al., 2007). Additional CPPs for use in the invention include without limitation, TAT protein transduction domain sequences (e.g. YGRKKRRQRRR (SEQ ID NO:72) or GRKKRRQRRRPPQ (SEQ ID NO:73) from HIV-1, angiopep (angiopepl: TFFYGGCRGKRNNFKTEEY; SEQ ID NO:74; angiopep-2: TFFYGGSRGKRNNFKTEEY; SEQ ID NO:75), transportan (CLIKKALAALAKLNIKLLYGASNLTWG; SEQ ID NO:76), rabies bifrus glycoprotein, and SynB. For further information on CPP-mediated delivery, see, e.g., Demeule et al., 2008, Identification and Design of Peptides as a New Drug Delivery System for the Brain. J. Pharmacol. Exp. Ther. 2008 March; 324(3):1064-72. doi: 10.1124/jpet.107.131318; Zou et al., 2013, Cell-Penetrating Peptide-Mediated Therapeutic Molecule Delivery into the Central Nervous System. Curr. Neuropharmacol. 11(2):197; Arranz-Gibert, P. et al., 2018, HAI Peptide and Backbone Analogs—Validation and Enhancement of Biostability and Bioactivity of BBB Shuttles. Sci. Rep. 8:17932. doi:10.1038/s41598-018-35938-8.

Coloma et al. described a chimeric antibody that binds human insulin receptor (HiR). The antibody avidly bound to isolated human brain capillaries and showed robust uptake into a living primate brain when administered intravenously to a Rhesus monkey. (Coloma et al., 2000, Pharm. Res. 17(3):266. The transferrin receptor (TfR) is another useful target which is highly expressed by brain endothelial cells. TfR1 is also well characterized among BBB transport receptors. Examples of peptides interacting with TfR1 include B6 (GGHKAKGPRKLGS; SEQ ID NO:77) (Xia et al., 2000, J. Virol 74(23):11359), THR (THRPPMWSPVWP; SEQ ID NO:78), and HAIYPRH (SEQ ID NO:79) (Lee et al., 2001, Eur. J. Biochem. 268(7):2004) and antibody binding domains that bind to TfR1 have been used for transcytosis. The peptides are useful for BBB transport of antibodies and antigen-binding fragments of the invention.

Ligands of RMT targets can be further engineered to enhance transport. For example, such Yu et al. described reducing the affinity of a ligand (an antibody) for the transferrin receptor (TfR) enhances receptor-mediated transcytosis across the BBB into the mouse brain where it reaches higher therapeutically relevant concentrations. Anti-TfR antibodies that bind with high affinity to TfR remained associated with the BBB, whereas lower-affinity anti-TfR antibody variants were released from the BBB into the brain and show a broad distribution 24 hours after dosing. (Yu et al., 2011, Boosting brain uptake of a therapeutic antibody by reducing its affinity for a transcytosis target. Sci. Trans. Med. 3(84):84ra44) Bien-Ky et al. described transport across the BBB of an antibody pertinent to AD. A bispecific antibody against TfR and β-secretase (β-amyloid cleaving enzyme-1; BACE1) crossed the BBB to reduce brain Aβ levels. (Bien-Ly, N. et al, 2014, Transferrin receptor (TfR) trafficking determines brain uptake of TfR antibody affinity variants. J. Exp. Med. 211(2):233).

Additional BBB targets useful in the invention include without limitation, basigin, Glut1, and CD98hc, and antibodies to each of these targets were significantly enriched in the brain after administration in vivo.targets (Zuchero et al., 2016, Discovery of novel blood-brain barrier targets to enhance brain uptake of therapeutic antibodies. Neuron 89(1):70). Table 2 provides further non-limiting examples of peptides useful in systems of the invention for delivery across the BBB (see Islam et al., Peptide based drug delivery systems to the brain. Nano Express vol. 1, No. 1).

TABLE 2
Exemplary Peptides to Target the Blood Brain Barrier

| | | | SEQ |
|---|---|---|---|
| | | | ID |
| Peptide | Typical Sequence | Origin | NO. |
| g7 | GFtGPLS (O-ß-d-glucose)CONH2 | Enkephalin | |
| | | analogues/opioid | |
| Apamin | H-CNCKAPETALCARRCQQH-NH2 | Venom neurotoxin | 88 |
| MiniAp-4 | [Dap]KAPETALD | Venom neurotoxin | 89 |
| Regulon | PTVIHGKREVTLHL | Neurotropic | 90 |
| polypeptides | | endogenous Protein | |
| RAP | ELKHFEAKIEKHNHYQKQLE | Neurotropic | 91 |
| | | endogenous Protein | |
| Angiopep-2 | TFFYGGSRGKRNNFKTEEY | Neurotropic | 75 |
| | | endogenous Protein | |
| TAT (47-57) | GGGGYGRKKRRQRRR | HIV Protein | 92 |
| RI-OR2- | Ac-rGffvlkGrrrrqrrkkrGy-NH2 | HIV Protein and | 93 |
| TAT | | Amyloid beta | |
| SynB1 | RGGRLSYSRRRFSTSTGR | Protegrins | 94 |
| Pep 22 | Ac-[cMPRLRGC]c-NH2 | Phage display | 95 |
| | | (receptor) | |
| Leptin 30 | YQQVLTSLPSQNVLQIANDLENLRDLLHLLC | Leptin | 96 |
| TGN | TGNYKALHPHNG | Phage display | 97 |
| LNP | KKRTLRKNDRKKRC | nucleolar | 98 |
| | | translocation signal | |
| | | sequence of LIM | |
| | | Kinase 2 | |
| ApoE | (LRKLRKRLLR)2 | Apolipoprotein E | 99 |
| (157-167) | | | |
| ApoB | SSVIDALQYKLEGTTRLTRKRGLKLATA | Apolipoprotein B | 66 |
| | LSLSNKFVEGS | | |
| RVG-29 | YTIWMPENPRPGTPCDIFTNSRGKRASNG | Rabies Virus | 100 |
| | | Glycoprotein | |
| G23 | HLNILSTLWKYRC | Phage display | 101 |
| T7 | HAIYPRH | Phage display | 79 |
| THR | THRPPMWSPVWP | Phage display | 78 |
| THRre | pwvpswmpprht (retro-enantio version | Phage display | 102 |
| | of THR) | | |
| THRre_2f | (pwvpswmpprht)2KKGK(CF)G | Branched-Phage | |
| | | display | |
| DKP | Phe(p-NH-Dhp)-L-N-Me[Cha]/[2Nal] | Unknown | |
| GSH-PEG | GSH[PEG] | Endogenous | |
| | | tripeptide | |
| CDX | D-[FKESWREARGTRIERG] | Structure-guided | 103 |
| | | design | |
| CRT | CRTIGPSVC | Phage display | 104 |
| T7-#2077 | RLSSVDSDLSGC | Phage display | 105 |
| CAQK | CAQK | Phage display | 106 |

### Nucleic Acid Molecules

The present invention further provides nucleic acid sequences that encode the antibodies described above.

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucl. Acids Res., 19:508 (1991); Ohtsuka et al., JBC, 260:2605 (1985); Rossolini et al., Mol. Cell. Probes, 8:91 (1994). A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

### Production and Delivery

Antibodies of the invention can be administered as nucleic acids to be expressed in a subject or produced externally and administered directly. It will be appreciated that nucleic acids encoding the antibodies are optimized depending on the location of production. Nucleic acids to be expressed in a subject (delivered for example by virus or liposomes) are engineered for expression and secretion in a cell of a subject, including selection of a suitable expression control sequences and a suitable signal peptide. Expression may be transient or constitutive.

In the examples herein, the signal peptide derived from the murine immunoglobulin kappa (IgGκ) light chain (METDTLLLWVLLLWVPGSTG; SEQ ID NO:80) was used to optimize viral transgene expression. (e.g., Fonseca et al., 2018, Inclusion of the murine IgGκ signal peptide increases the cellular immunogenicity of a simian adenoviral vectored Plasmodium vivax multistage vaccine. Vaccine 36(20):2799; Wang et al, 2016, Efficient production of CYTL1 protein using mouse IgGκ signal peptide in the CHO cell expression system. Acta Biochim Biophys Sin 48(4), 391).

The invention includes administration of nucleic acids to humans and non-human primates, hence expression in human and non-human primate cells. Further, the invention includes ex vivo production of antibodies for administration to a patient, production which can be in mammalian cells, including human and non-human cells, as well as non-mammalian cells such as bacterial cells. Secretion signal peptides are selected accordingly and the encoding nucleic acid may be codon optimized.

In some embodiments, nucleic acids encoding and expressing antibodies of the invention are comprised within a delivery system, optionally: a vector system comprising one or more vectors, optionally wherein the vectors comprise one or more viral vectors, optionally wherein the one or more viral vectors comprise one or more lentiviral, adenoviral or adeno-associated viral (AAV) vectors; or a particle or lipid particle.

In some embodiments, nucleic acids encoding and expressing antibodies of the invention comprise mRNA. The introduction of chemical modifications to mRNA, such as 5′ cap, 5′- and 3′-UTRs, nucleic acid analogs, coding region optimization, and the poly(A) tail, has overcome obstacles such as low levels of expression and immunogenicity to the point where mRNA is successfully applied for immunization and cancer immunotherapy. mRNA is delivered by co-formulation into lipid nanoparticles (LNPs). LNP formulations are typically composed of (1) an ionizable or cationic lipid or polymeric material (2) a zwitterionic lipid (e.g., 1,2-dioleoyl-sn-glycero-3-phosphoethanolamine [DOPE]) that resembles the lipids in the cell membrane; (3) cholesterol to stabilize the lipid bilayer of the LNP; and (4) a polyethylene glycol (PEG)-lipid to lend the nanoparticle a hydrating layer, improve colloidal stability, and reduce protein absorption. See, e.g., Kowalski et al., 2019, Delivering the Messenger: Advances in Technologies for Therapeutic mRNA Delivery. Mol. Ther. 27(4):710.

Several approaches exist for delivering molecules across the blood brain barrier (BBB) such as alteration of the administration route, disruption of the BBB and alteration of its permeability, nanoparticle delivery, Trojan horse approaches, receptor-mediated transport, and cell and gene therapy.

Alteration of the administration route can be achieved by direct injection into the brain (see, e.g., Papanastassiou et al., Gene Therapy 9: 398-406(2002)), implanting a delivery device in the brain (see, e.g., Gillet al., Nature Med. 9: 589-595 (2003); and Gliadel Wafers™, Guildford Pharmaceutical), and intranasal administration to bypass the BBB (Mittal et al, Drug Deliv. 21(2):75-86. (2014))

Methods of barrier disruption include, but are not limited to, ultrasound (see, e.g., U.S. Patent Publication No. 2002/0038086), osmotic pressure (e.g., by administration of hypertonic mannitol (Neuwelt, E. A., Implication of the Blood-Brain Barrier and its Manipulation, Vols 1 & 2, Plenum Press, N.Y. (1989))), permeabilization by, e.g., bradykinin or permeabilizer A −7 (see, e.g., U.S. Pat. Nos. 5,112,596, 5,268,164, 5,506,206, and 5,686,416).

Methods of altering the BBB permeability include, but are not limited to, using glucocorticoid blockers to increase permeability of the Blood-Brain Barrier (see, e.g., U.S. Patent Application Publication Nos.

2002/0065259, 2003/0162695, and 2005/0124533); activating potassium channels (see, e.g., U.S. Patent Application Publication No. 2005/0089473), and inhibiting ABC drug transporters (see, e.g., U.S. Patent Application Publication No. 2003/0073713).

### Viral Vectors

In an aspect, the invention provides for production of antibodies by host cells, Non-limiting examples include viral transfection and mRNA transfection. According to the invention, the viral vector can be an adeno-associated virus (AAV) or pseudotype or mutant thereof. In certain embodiments, the AAV is an AAV1, AAV2, AAV3, AAV4, AAV5, AAV6, AAV7, AAV8, AAV9, AAV10, AAV11, AAV2/5, or AAV-DJ. In certain embodiments, the AAV is an AAV pseudotype, including but not limited to AAV2/5, AAV2/7 of AAV2/8. In certain embodiments, the AAV comprises a hybrid capsid, including but not limited to AAV-DJ. In certain embodiments, the AAV comprises a mutant capsid, including but not limited to AAV2/8(Y733F), AAV2/2(quad Y-F) and AAV2/2(7m8), or AAV-DJ8.

One can select the AAV of the AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells (see Grimm, D. et al, J. Virol. 82: 5887-5911 (2008)) is as follows:

| Cell Line | AAV-1 | AAV-2 | AAV-3 | AAV-4 | AAV-5 | AAV-6 | AAV-8 | AAV-9 |
|---|---|---|---|---|---|---|---|---|
| Huh-7 | 13 | 100 | 2.5 | 0.0 | 0.1 | 10 | 0.7 | 0.0 |
| HEK293 | 25 | 100 | 2.5 | 0.1 | 0.1 | 5 | 0.7 | 0.1 |
| HeLa | 3 | 100 | 2.0 | 0.1 | 6.7 | 1 | 0.2 | 0.1 |
| HepG2 | 3 | 100 | 16.7 | 0.3 | 1.7 | 5 | 0.3 | ND |
| Hep1A | 20 | 100 | 0.2 | 1.0 | 0.1 | 1 | 0.2 | 0.0 |
| 911 | 17 | 100 | 11 | 0.2 | 0.1 | 17 | 0.1 | ND |
| CHO | 100 | 100 | 14 | 1.4 | 333 | 50 | 10 | 1.0 |
| COS | 33 | 100 | 33 | 3.3 | 5.0 | 14 | 2.0 | 0.5 |
| MeWo | 10 | 100 | 20 | 0.3 | 6.7 | 10 | 1.0 | 0.2 |
| NIH3T3 | 10 | 100 | 2.9 | 2.9 | 0.3 | 10 | 0.3 | ND |
| A549 | 14 | 100 | 20 | ND | 0.5 | 10 | 0.5 | 0.1 |
| HT1180 | 20 | 100 | 10 | 0.1 | 0.3 | 33 | 0.5 | 0.1 |
| Monocytes | 1111 | 100 | ND | ND | 125 | 1429 | ND | ND |
| Immature DC | 2500 | 100 | ND | ND | 222 | 2857 | ND | ND |
| Mature DC | 2222 | 100 | ND | ND | 333 | 3333 | ND | ND |

Lentiviral vectors can also employed for delivery. Lentiviral vectors are useful for delivery to the brain, see, e.g., US Patent Publication Nos. US20110293571; US20110293571, US20040013648, US20070025970, US20090111106 and U.S. Pat. No. 7,259,015, including in treatment for Parkinson's Disease, see, e.g., US Patent Publication No. 20120295960 and U.S. Pat. Nos. 7,303,910 and 7,351,585. Lentiviral vectors have also been disclosed for the treatment of ocular diseases, see e.g., US Patent Publication Nos. 20060281180, 20090007284, US20110117189; US20090017543; US20070054961, US20100317109. Lentiviral vectors are used accordingly for delivery of antibodies of the invention.

### Liposomes

Delivery or administration according to the invention can be performed with liposomes. Liposomes are spherical vesicle structures composed of a uni- or multilamellar lipid bilayer surrounding internal aqueous compartments and a relatively impermeable outer lipophilic phospholipid bilayer. Liposomes have gained considerable attention as drug delivery carriers because they are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

In one embodiment, Trojan Horse liposomes (also called pegylated immunoliposomes) are used. These particles allow delivery of nucleic acids to the entire brain after an intravascular injection. Without being bound by limitation, it is believed that lipid particles with specific antibodies conjugated to surface allow crossing of the blood brain barrier via endocytosis. See Pardridge, 2020, Brain Delivery of Nanomedicines: Trojan Horse Liposomes for Plasmid DNA Gene Therapy of the Brain. Front. Med. Technol. vol. 2, article 602236.

Zhang et al. (Mol Ther. 2003 January; 7(1):11-8.)) describe how expression plasmids encoding reporters such as luciferase were encapsulated in the interior of an “artificial virus” comprised of an 85 nm pegylated immunoliposome, which was targeted to the rhesus monkey brain in vivo with a monoclonal antibody (MAb) to the human insulin receptor (HIR). The HIRMAb enables the liposome carrying the exogenous gene to undergo transcytosis across the blood-brain barrier and endocytosis across the neuronal plasma membrane following intravenous injection.

The terms “protein,” “peptide” and “polypeptide” are used interchangeably herein.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified. The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

“Wild-type” refers to the normal gene, or organism found in nature without any known mutation.

“Genome” refers to the complete genetic material of an organism.

A “vector” is defined to include, inter alia, any plasmid, cosmid, phage or binary vector in double or single stranded linear or circular form which may or may not be self transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al., Mol. Biotech., 3:225 (1995).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position +1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

“Constitutive expression” refers to expression using a constitutive or regulated promoter. “Conditional” and “regulated expression” refer to expression controlled by a regulated promoter.

“Operably-linked” refers to the association of nucleic acid sequences on single nucleic acid fragment so that the function of one is affected by the other. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The terms “cis-acting sequence” and “cis-acting element” refer to DNA or RNA sequences whose functions require them to be on the same molecule.

The terms “trans-acting sequence” and “trans-acting element” refer to DNA or RNA sequences whose function does not require them to be on the same molecule.

The following terms are used to describe the sequence relationships between two or more nucleic acids or polynucleotides: (a) “reference sequence,” (b) “comparison window,” (c) “sequence identity,” (d) “percentage of sequence identity,” and (e) “substantial identity.”

(a) As used herein, “reference sequence” is a defined sequence used as a basis for sequence comparison. A reference sequence may be a subset or the entirety of a specified sequence; for example, as a segment of a full length cDNA or gene sequence, or the complete cDNA or gene sequence.

(b) As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller, CABIOS, 4:11 (1988); the local homology algorithm of Smith et al., Adv. Appl. Math., 2:482 (1981); the homology alignment algorithm of Needleman and Wunsch, JMB, 48:443 (1970); the search-for-similarity-method of Pearson and Lipman, Proc. Natl. Acad. Sci. USA, 85:2444 (1988); the algorithm of Karlin and Altschul, Proc. Natl. Acad. Sci. USA, 87:2264 (1990), modified as in Karlin and Altschul, Proc. Natl. Acad. Sci. USA, 90:5873 (1993).

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al., Gene, 73:237 (1988); Higgins et al., CABIOS, 5:151 (1989); Corpet et al., Nucl. Acids Res., 16:10881 (1988); Huang et al., CABIOS, 8:155 (1992); and Pearson et al., Meth. Mol. Biol., 24:307 (1994). The ALIGN program is based on the algorithm of Myers and Miller, supra. The BLAST programs of Altschul et al., JMB, 215:403 (1990); Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al., Nucleic Acids Res. 25:3389 (1997). Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of nucleotide sequences for determination of percent sequence identity to the promoter sequences disclosed herein is preferably made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, at least 90%, at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution. For DNA-DNA hybrids, the Tₘ can be approximated from the equation of Meinkoth and Wahl, Anal. Biochem., 138:267 (1984); Tₘ 81.5° C.+16.6 (log M)+0.41 (% GC)− 0.61 (% form)− 500/L; where M is the molarity of monovalent cations, % GC is the percentage of guanosine and cytosine nucleotides in the DNA, % form is the percentage of formamide in the hybridization solution, and L is the length of the hybrid in base pairs. Tₘ is reduced by about 1° C. for each 1% of mismatching; thus, Tₘ, hybridization, and/or wash conditions can be adjusted to hybridize to sequences of the desired identity. For example, if sequences with >90% identity are sought, the Tₘ can be decreased 10° C. Generally, stringent conditions are selected to be about 5° C. lower than the Tₘ for the specific sequence and its complement at a defined ionic strength and pH. However, severely stringent conditions can utilize a hybridization and/or wash at 1, 2, 3, or 4° C. lower than the Tₘ; moderately stringent conditions can utilize a hybridization and/or wash at 6, 7, 8, 9, or 10° C. lower than the Tₘ; low stringency conditions can utilize a hybridization and/or wash at 11, 12, 13, 14, 15, or 20° C. lower than the Tₘ. Using the equation, hybridization and wash compositions, and desired temperature, those of ordinary skill will understand that variations in the stringency of hybridization and/or wash solutions are inherently described. If the desired degree of mismatching results in a temperature of less than 45° C. (aqueous solution) or 32° C. (formamide solution), it is preferred to increase the SSC concentration so that a higher temperature can be used. An extensive guide to the hybridization of nucleic acids is found in Tijssen, Laboratory Techniques in Biochemistry and Molecular Biology Hybridization with Nucleic Acid Probes, part I chapter 2 “Overview of principles of hybridization and the strategy of nucleic acid probe assays” Elsevier, New York (1993). Generally, highly stringent hybridization and wash conditions are selected to be about 5° C. lower than the Tₘ for the specific sequence at a defined ionic strength and pH.

An example of highly stringent wash conditions is 0.15 M NaCl at 72° C. for about 15 minutes. An example of stringent wash conditions is a 0.2×SSC wash at 65° C. for 15 minutes (see, Sambrook, infra, for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example low stringency wash for a duplex of, e.g., more than 100 nucleotides, is 4-6×SSC at 40° C. for 15 minutes. For short probes (e.g., about 10 to 50 nucleotides), stringent conditions typically involve salt concentrations of less than about 1.5 M, more preferably about 0.01 to 1.0 M, Na ion concentration (or other salts) at pH 7.0 to 8.3, and the temperature is typically at least about 30° C. and at least about 60° C. for long probes (e.g., >50 nucleotides). Stringent conditions may also be achieved with the addition of destabilizing agents such as formamide. In general, a signal to noise ratio of 2× (or higher) than that observed for an unrelated probe in the particular hybridization assay indicates detection of a specific hybridization. Nucleic acids that do not hybridize to each other under stringent conditions are still substantially identical if the proteins that they encode are substantially identical. This occurs, e.g., when a copy of a nucleic acid is created using the maximum codon degeneracy permitted by the genetic code.

Very stringent conditions are selected to be equal to the Tₘ for a particular probe. An example of stringent conditions for hybridization of complementary nucleic acids which have more than 100 complementary residues on a filter in a Southern or Northern blot is 50% formamide, e.g., hybridization in 50% formamide, 1 M NaCl, 1% SDS at 37° C., and a wash in 0.1×SSC at 60 to 65° C. Exemplary low stringency conditions include hybridization with a buffer solution of 30 to 35% formamide, 1M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel, Proc. Natl. Acad. Sci. USA, 82:488 (1985); Kunkel et al., Meth. Enzymol., 154:367 (1987); U.S. Pat. No. 4,873,192; Walker and Gaastra, Techniques in Mol. Biol. (MacMillan Publishing Co. (1983), and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. The deletions, insertions, and substitutions of the polypeptide sequence encompassed herein are not expected to produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “transformed,” “transformant,” and “transgenic” cells have been through the transformation process and contain a foreign gene integrated into their chromosome. The term “untransformed” refers to normal cells that have not been through the transformation process.

A “transgenic” organism is an organism having one or more cells that contain an expression vector.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

### Formulations and Methods of Administration

For in vivo use, a therapeutic agent as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

In certain embodiments, the present therapeutic agent may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of the present invention can be determined by comparing their in vitro activity, and in vivo activity in animal models. In certain embodiments, a useful dose is from about 0.1 mg/kg to about 5 mg/kg or from about 0.5 mg/kg to about 2 mg/kg. Methods for the extrapolation of effective dosages in humans and animals of different sizes are known to the art; for example, see U.S. Pat. No. 4,938,949 and Nair et al., A simple practice guide for dose conversion between animals and human. J. Basic Clin. Pharm. March 2016-May 2016; 7(2): 27-31. doi: 10.4103/0976-0105.177703.

Generally, the concentration of the compound(s) of the present invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Materials and Methods

C6T and A4 scFvs

The A4 scFv binds an oligomeric Aβ variant with an average particle height of ˜3.6 nm and a diameter ˜10 nm (17, 21), consistent with the size of a dodecamer (13, 19), whereas C6T binds an oligomeric Aβ variant with an average particle height ˜2.1 nm and diameter ˜5 nm, consistent with the size of tri/tetrameric Aβ (18) (Table 3).

TABLE 3
Comparison of targets identified by Aβ conformation specific scRvs A4 and C6T

| | scFv-A4 | scFv-C6T |
|---|---|---|
| Antigen epitope | Synthetic Aβ1-42 aggregate | Human-derived oligomeric Aβ |
| Phage Library | Tomlinson I + J (Winter et al., 1994) | Sheets (Sheets et al., 1998) |
| Aβ aggregate height | ~3.6 nm | ~2.1 nm |
| Aβ aggregate diameter | ~10 nm | ~5 nm |
| Aβ fragment number | ~12-mers (dodecamer) | ~3-4 mers (tri/tetramer) |

The aggregate sizes were measured with the height of size under AFM and expressed as an average size by scale nanometer.

### Construction of Recombinant Human Adenovirus Vectors.

As described previously (22), the cDNA constructs of the C6T and A4 scFv genes were amplified by polymerase chain reaction (PCR) and then cloned into pFBAAVCAGmcsBgHpA vector (G0345, viral vector core facility, University of Iowa, Iowa City, Iowa). The tag FLAG was placed at the C-terminal of the scFv for use as a marker. The vectors AAV2/8 containing plasmids pAAV-scFv were generated through triple transfection into HEK293 cells (Viral Vector Core Facility, University of Iowa, Iowa City, Iowa). After cellular infection, the vectors encoding either the A4 or C6T scFv along with C-terminal apolipoprotein B (ApoB) tag were verified to bind the respective conformations of Aβ aggregates. A control rAAV encoding green fluorescent protein (GFP) without ApoB of FLAG was prepared similarly to the scFv constructs. The release of viral particles containing the vector genomes were measured and the titers of rAAV virions were determined by a quantitative dot-blot assay (Viral Vector Core Facility, University of Iowa, Iowa City, Iowa).

Gene and amino acid sequences of the A4 AAV and c6T AAV vectors have the design Kozak-IgK-SacI-ApoB-NcoI-scFv-NotI-FLAG-Stop as shown below. In the amino acid sequences, ApoB and scFv sequences are underlined to distinguish from secretion signal peptides, linker amino acids, and tags.

A4 AAV construct with tags (SEQ ID NO: 83):
gccgccAcAATGgAtggagacagacacactcctgctatgggtactgctgctctgggttccaggttccactggtgacGAGCTCtc
atctgtcattgatgcactccaatacaaattagagggcaccacaagattgacaagaaagagaggattgaagttagccacagctctgtctctgag
caacaaatttgtggagggtagtggtggaggcggatctgCCATGGccgaggtgcagctgttggagtctgggggaggcttggtacagcc
tggggggtccctgagactctcctgtgcagcctctggattcacctttagcagctatcccatgagctgggtccgccaggctccagggaagggg
ctggagtgggtctcagcgattcagcatactggtgcgccgacaacttacgcagactccgtgaagggccggttcaccatctccagagacaatt
ccaagaacacgctgtatctgcaaatgaacagcctgagagccgaggacacggccgtatattactgtgcgaaagcgtttccgccgtttgactac
tggggccagggaaccctggtcaccgtctcgagcggtggaggcggttcaggcggaggtggcagcggcggtggcgggtcgacggacatc
cagatgacccagtctccatcctccctgtctgcatctgtaggagacagagtcaccatcacttgccgggcaagtcagagcattagcagctattta
aattggtatcagcagaaaccagggaaagcccctaagctcctgatctattctgcatcctctttgcaaagtggggtcccatcaaggttcagtggc
agtggatctgggacagatttcactctcaccatcagcagtctgcaacctgaagattttgcaacttactactgtcaacagcgggagactgggcct
aaggcGttcggCcaaGggaCcaaGgtggaaatcaaacggGCGGCCGCAgattacaaggatgacgatgacaagTGA
ApoB-A4scFv with IgK signal peptide and FLAG tag (SEQ ID NO: 84):
METDTLLLWVLLLWVPGSTGDELSSVIDALQYKLEGTTRLTRKRGLKLATALSLSNKFVEGSGG
GGSAMAEVQLLESGGGLVQPGGSLRLSCAASGFTFSSYPMSWVRQAPGKGLEWVSAIQHTGAPT
TYADSVKGRFTISRDNSKNTLYLQMNSLRAEDTAVYYCAKAFPPFDYWGQGTLVTVSSGGGGSG
GGGSGGGGSTDIQMTQSPSSLSASVGDRVTITCRASQSISSYLNWYQQKPGKAPKLLIYSASSL
QSGVPSRFSGSGSGTDFTLTISSLOPEDFATYYCQQRETGPAFGQGTKVEIKRAAADYKDDDDK
c6T AAV construct with tags (SEQ ID NO: 81):
GTCGACgccgccAcAATGgAtggagacagacacactcctgctatgggtactgctgctctgggttccaggttccactggtgacG
AGCTCtcatctgtcattgatgcactccaatacaaattagagggcaccacaagattgacaagaaagagaggattgaagttagccacagct
ctgtctctgagcaacaaatttgtggagggtagtggtggaggcggatctgccatggcccaggtacagctgcaggagtcTgggggaggctt
ggtacagcctggggggtccctgagactctcctgtgcagcctctggattcacctttagcagctatgccatgagctgggtccgccaggctccag
ggaaggggctggagtgggtctcagctattagtggtagtggtggtagcacatactacgcagactccgtgaagggccgattcaccatctccag
agacaattccaagaacacgctgtatctgcaaatgaacagcctgagagctgaggacacggctgtgtattactgtgcgaagagctatggttcag
ttaaaataagctgctttgactactggggccagagcaccctggtcaccgtctcctcaggtggaggcggttcaggcggaggtggctctggcgg
tggcggatcggaaattgtgctgacgcagtctccagactccctggctgtgtctctgggcgagagggccaccatcaactgcaagtccagccag
agtgttctttacaactccaacaataagaactacttagcttggtaccagcagaaaccaggacagtctcctgagttgctcatttactgggcatca
acccgggaatccggggtccctgaccgattcagtggcagcgggtctgggacagaattcactcttaccatcagcagcctgcaggctgaggatgt
ggcagtttattactgtcagcaattttatagtactcctccgacttttggccaggggaccaagctggagatcaaacgtGCGGCCGCAgatt
acaaggatgacgatgacaagTGA
ApoB-c6TscFv with IgK signal peptide and FLAG tag (SEQ ID NO: 82):
METDILLLWVLLLWVPGSTGDELSSVIDALQYKLEGTTRLTRKRGLKLATALSLSNKFVEGSGG
GGSAMAQVQLQESGGGLVQPGGSLRLSCAASGFTFSSYAMSWVRQAPGKGLEWVSAISGSGGST
YYADSVKGRFTISRDNSKNTLYLQMNSLRAEDTAVYYCAKSYGSVKISCFDYWGQSTLVTVSSG
GGGSGGGGSGGGGSEIVLTQSPDSLAVSLGERATINCKSSQSVLYNSNNKNYLAWYQQKPGQSP
ELLIYWASTRESGVPDRFSGSGSGTEFTLTISSLQAEDVAVYYCQQFYSTPPTFGQGTKLEIKR
AAADYKDDDDK-

### Human Samples

Human brain tissues were obtained from Dr. Thomas Beach, the Brain and Body Donation Program at Banner/Sun Health Research Institute (BBDP) (55, 56). The average age of the AD and ND subjects was 76.8±8.8, 85.2±8.5 years old, respectively. The neuropathological characteristics are shown in Table 4. The postmortem AD and cognitively normal controls (ND) utilized were confirmed pathologically. The neocortex of superior frontal cortex was sectioned with 40 μm and immunolabeling was described in following immunofluorescence staining.

TABLE 4
The pathological profiles of human subjects
from the brains with AD and controls.

| | | | | Postmortem | Disease | |
|---|---|---|---|---|---|---|
| | Age | | | interval | duration | |
| Gender | (years) | Diagnosis | MMSE | (hours) | (years) | ApoE |
| M | 77 | AD | NA | 2.33 | 12 | 3/4 |
| M | 81 | AD | 6 | 3 | 13 | 3/4 |
| F | 89 | AD | 6 | 2.33 | 9 | 3/4 |
| M | 67 | AD | 13 | 2 | 8 | 3/4 |
| M | 70 | AD | 6 | 2.33 | 11 | 3/4 |
| M | 81 | ND | 25 | 2.75 | | 3/3 |
| F | 90 | ND | 27 | 3 | | 3/3 |
| F | 83 | ND | 29 | 3.25 | | 3/3 |
| M | 97 | ND | NA | 1.5 | | 2/3 |
| F | 75 | ND | NA | 2.75 | | 3/4 |

ND, non-demented control; AD, Alzheimer's disease; M, male; F, female.

### Animals

One month old female APP/PS1 mice (MMRRC 34832) and age matched wild type mice were purchased from Jackson Laboratory (Bar Harbor, ME). The transgenic mouse is generated on a genetic background C57BL/6J expressing Swedish mutation KM670/671NL of human amyloid precursor protein (APPswe) and a presenilin 1 lacking exon 9 (PSEN1dE9) (27). After 6-8 weeks, the transgenic mice showed cerebral amyloidosis and amyloid-associated pathologies including dystrophic synaptic buttons, robust gliosis, and increase in microglia number increase and activation (29). All protocols for animal use here were approved by the Institutional Animal Care and Use Committee (IACUC) (Arizona State University, Tempe, Arizona). Animals were treated in accordance with good animal practice following NIH requirements.

Administration of rAAV-scFvs

Two month old APP/PS1 mice were randomly assigned into one of three groups: two test groups receiving either rAAV-C6T (n=10) or rAAV-A4 (n=12), or a positive vehicle control group rAAV-GFP (n=20). The negative control received rAAV-GFP in age-matched WT (n=15). All of the mice were subjected to a single intraperitoneal injection of rAAV-scFv or rAAV-GFP at a dosage of 3.0×10¹⁰ vector genomes (vg)/mouse essentially as described. (He, Xin, 2019)

### Tissue Harvest.

Seven months after the injection, surviving mice were euthanized with isoflurane and perfused with 0.1 M phosphate-buffer (PB) including 10 U of heparin (AK3004, Akron Biotech, Boca Raton, FL). Survival numbers for each group were: Tg-C6T (n=9), Tg-A4 (n=7), Tg-GFP mice (n=12) and WT (n=15). The mouse brain tissues were quickly harvested. The right hemispheres were stored at −80° C. for biochemistry and the left ones were fixed in 4% (w/v) paraformaldehyde (PFA) for histology.

### Cell Culture and Immunofluorescence.

HEK293 cells stably transfected with the Swedish mutation (K595N/M596L) of human amyloid precursor protein (APPswe) (gift from Dr. Hailan Yao from Roskamp Institute, Sarasota, FL) were maintained in Dulbecco's modified Eagle's medium with 10% fetal bovine serum (FBS) supplemented with G418 sulfate 0.6 mg/ml (Geneticin, Catalog:10131035, Gibco, Thermo Fisher Scientific). Untransfected HEK293 cells were cultured in 10% FBS without G418, as controls. The cells were seeded on a glass chamber with 0.5% FBS for 24 h and then fixed with 4% paraformaldehyde for 10 min. The nonspecific staining was blocked with 10% goat serum for 30 min. The cells were incubated with purified A4 or C6T scFv. Then antibodies against c-Myc tagged scFv (Sigma, 1:1000) and Aβ1-17 (6E10, Covance, 1:1000) were applied. Total A and the C6T- and A4-recognized oligomeric variants of Aβ were visualized with fluorescent-labeled secondary antibodies (1:1000, Invitrogen, Thermo Fisher Scientific).

### Enzyme-Linked Immunosorbent Assay

Samples were homogenized using homogenization buffer which contains 1% Nonidet P-40 (Calbiochem, Billerica, MA), and protease and phosphatase inhibitor cocktails (Roche, Pleasanton, CA). The homogenate was centrifuged at 14,000 rpm for 20 min. The protein concentration of supernatants was measured with a Pierce™ bicinchoninic acid (BCA) protein assay kit (Thermo Scientific, Rockford, IL) as total protein values. The sandwich ELISA using the phage particles for detection was used as described previously (25, 26). Briefly, the capture scFv was immobilized to the wells of a high binding 96-well ELISA plate (Costar, USA) and any unbound sites blocked with 2% milk-PBS. Homogenized samples were added to the wells for 2 hours specific binding. After washing, homogenized samples were incubated for 2 hour at 37° C. followed by 200 ng/ml of a 40 mM carboxyl biotinylated detection phage. Any bound biotinylated phage was identified using the avidin-HRP antibody. Following addition of the SuperSignal ELISA Femto Maximum Sensitivity Substrate (Thermo Scientific, USA), signal intensities were quantified using the Wallac Victor² microplate reader. After each incubation step, the wells were washed 3-4 times with 0.1% PBS-Tween20 to reduce non-specific binding.

### Chromogenic DAB Immunostaining

The brain sections were prepared as described previously (25, 55). In brief, the brain tissues were sagittally sectioned 30 μm thick with a Cryostat (Leica, CM3000). Immunostaining was performed as described previously (22, 34, 57). Briefly, after blocking non-specific protein binding, the sections were then incubated with a monoclonal antibody 6E10 (catalog: SIG-39320, Covance, 1:2000) and monoclonal antibody against glial fibrillary acidic protein (GFAP, catalog: SMI-22R, Covance, 1:5000). Then sections were incubated with biotinylated secondary antibody of horse anti-mouse IgG. Following washing, the Vectastain kit (Vector Laboratories, Burlingame, CA) was applied. Samples were visualized using 3,3′-diaminobenzidine (DAB) as a substrate (Vector Laboratories). The sections were processed deleting primary antibody as negative controls and counterstained with haematoxylin (Sigma-Adrich, St. Louis, MO).

### Congo Red Staining

Fibrillar deposits were visualized with histological staining of Congo-Red (C6T277, Sigma-Aldrich, St. Louis, MO) (22). Prior to incubation, the solution was filtered. After the incubation of Congo-Red for 20 min, the sections were counterstained with hematoxylin to show cell nuclei (Sigma-Aldrich, St. Louis, MO).

### Immunofluorescent Staining

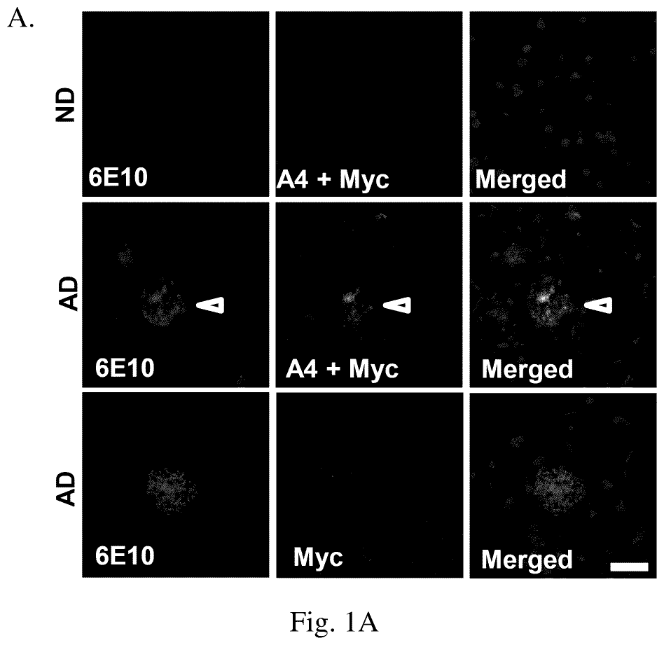

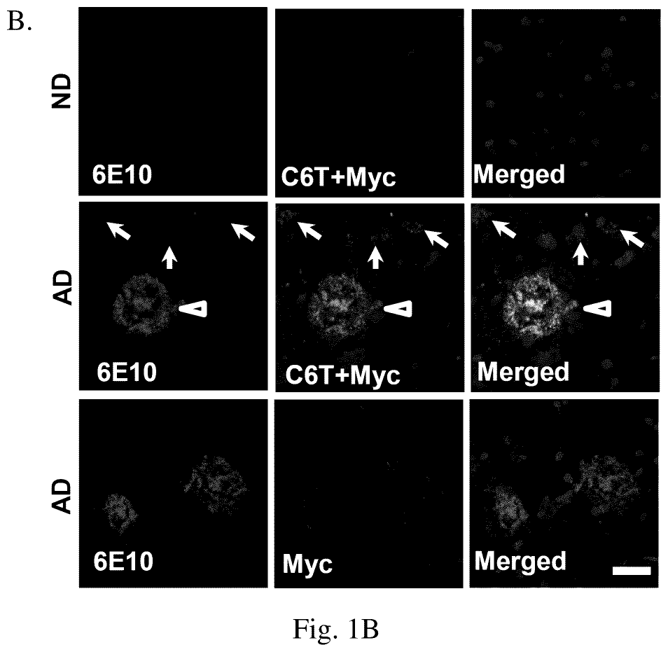

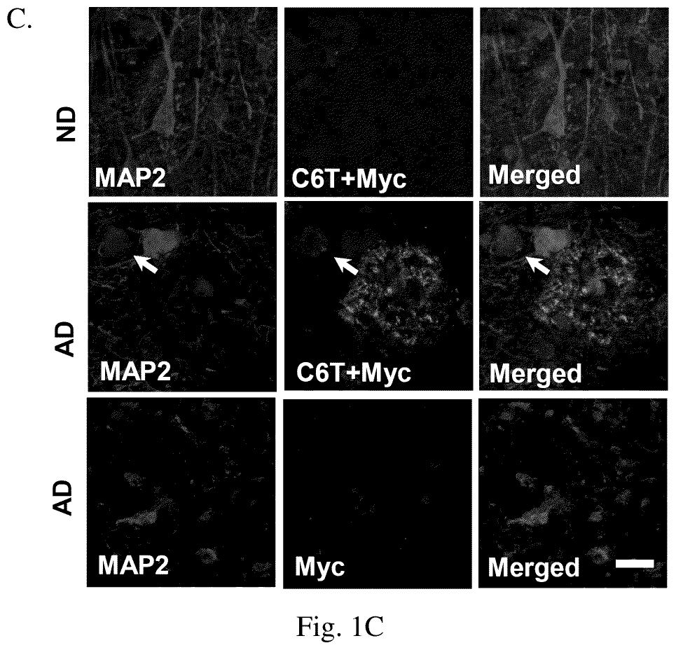

Immunofluorescent staining of oligomeric Aβ by C6T and A4 was performed as follows. In brief, the human or mouse brain sections were incubated with purified scFv antibody A4 and C6T (1 ug/ml) at 4° C. overnight. Then a rabbit anti-cMyc antibody (C3956, Sigma-Aldrich, 1:1000) was used to detect the cMyc tag on the C6T and A4 scFvs. Images were double stained with antibody 6E10 to label Aβ (SIG-39320, Covance, 1:2000 dilution) or an antibody against MAP2 as a neuronal marker (MMS-485P, Covance, 1:500 dilution). Sudan Black was used for quenching non-specific fluorescence of intracellular lipofuscin. A control using secondary antibody against tag-Myc without the corresponding primary scFv was also performed (FIG. 1). Microglial cells were visualized with rabbit anti-ionized calcium binding adaptor molecule 1 (Iba1) antibody (catalog: 019-19741, Wako, Richmond, VA, 1:500). The synapses were immunostained using a rabbit antibody against synaptophysin (Santa Cruz, 1:100) and newborn neurons in the hippocampus were labeled with rabbit anti-doublecortin (DCX, Ab18723, Abcam, 1:500). Fluorescent conjugated secondary antibodies were used to visualize the target structures. The endogenous lipofuscin, was blocked with 0.3% Sudan Black and cell nuclei were labeled with 4′,6-diamidino-2-phenylindole (DAPI) (Electric Microscopy Sciences).

To detect AAV and scFv expression, sections of liver and brain tissue samples were incubated with antibodies against adeno-associated virus capsid VP3 (AAV2, Novus Biologicals, Centennial, CO) and against FLAG (Sigma-Aldrich, St. Louis, MO). Sudan black was applied to block the non-specific fluorescence of the endogenous lipofuscin. Cell nuclei were labeled with DAPI. The localization of GFP was directly observed and the images were captured by the confocal Leica TCS SP5 (Biodesign Institute, Arizona State University, AZ).

### Quantification of Immunostaining Structures

The quantification of immunostaining structures were performed as described previously (22). A microscope BX51T-PHD-J11 (Olympus, Tokyo, Japan) was utilized to capture images. The Image J analysis software (National Institute of Health, Bethesda, USA) was employed to analyze the structures of interest. A semi-automated red-green-blue color threshold was set to determine the optimal segmentation of threshold setting. The background counterstain was subtracted with a deconvolution method to determine the specific mean immunostaining. An average value of immunostaining areas was expressed as a percentage of total area in the cortex or the hippocampus. An average value was determined for each group and data were expressed as mean±SEM (standard error of the mean).

### ScFv Levels in Brain Tissue.

Fresh cortical tissues were harvested and homogenized in homogenization buffer. A designed fusion protein, Multiple Tag, containing the FLAG-tag sequence DYKDDDDK (Catalog: M0101, GenScript, Piscataway, NJ), with a molecular weight of ˜40 kDa on SDS-PAGE was used as a standard for calculating scFv concentration. The molecular weight of the tagged A4 and C6T scFvs were estimated as 30 KDa. A monoclonal antibody against FLAG-tag (Sigma-Aldrich St. Louis, MO) was applied. The protein assay was performed with ELISA method. The readout values of scFv proteins were normalized to pg/mg of brain tissues.

### Immunoblot Analysis

Mouse cortex tissue was homogenized in PBS buffer (Sigma-Aldrich, St. Louis, MO), supplemented with 1% Nonidet P-40 (Calbiochem, Billerica, MA), and protease and phosphatase inhibitor cocktails (Roche, Pleasanton, CA). The homogenate was centrifuged at 14,000 rpm for 20 min. The protein concentration was measured with a BCA protein assay kit (Pierce Biotechnology, Rockford, IL). The supernatants (50 μg) mixed with 2× Laemmli sample buffer (Bio-Rad, Hercules, CA) with 0.001% bromophenol blue were heated at 75° C. for 10 min and loaded to 8% SDS-PAGE. The separated proteins were electrotransferred to nitrocellulose membranes (Millipore, Bedford, MA). The membranes were blocked with 5% dry milk and were incubated with primary antibodies overnight: rabbit anti-N-terminal BACE1 (B0681, clone: 46-62, Sigma-Aldrich), mouse anti-human soluble β APP (Swedish mutation) (sAPPβ, catalog: 10321, Clone: A61, IBL-America). C-terminal fragment of APP were detected using rabbit polyclonal antibody (catalog: 171610, clone: 751-770, Sigma Aldrich). The membrane was then incubated with the secondary antibodies, goat anti-mouse or rabbit IgG conjugated with horseradish peroxidase (Santa-Cruz Biotechnology, Santa Cruz, CA) and visualized by an enhanced DAB system (Sigma-Aldrich) following the manufacturers' instructions. Semi-quantification analysis was performed using a Versadoc XL imaging apparatus (Bio-Rad). β actin (catalog: A1978; clone AC-15, Sigma-Aldrich) levels were used as loading controls.

### Statistical Analysis

Statistical analyses were performed as described previously (22) using SPSS 13.0 software (SPSS, Inc., Chicago, IL, USA). In order to determine the significant intergroup difference, two-way repeated measures of analysis of variance (ANOVA) and Student Newman-Keuls-q or Dunnet t-test were applied. A statistical significance was considered as the value of p <0.05.

### Example 2

Localization of Oligomeric Aβ Variants by C6T and A4 scFvs in Human AD Brains.

Two scFv fragments, A4 and C6T, were isolated against two conformationally distinct oligomeric Aβ species, one synthetically generated, and one AD brain derived (Table 3) (17, 18, 21). A4- and C6T-recognized oligomeric Aβ variants can both be detected in human brain tissue (18), cerebrospinal fluid (25) as well as sera samples (25, 26). Brain sections of post-mortem human AD and control cases were immunostained with purified A4 and C6T scFvs. Results showed little expression of A4- and C6T-recognized oligomeric variants in the age matched non-dementia (ND) brains (FIG. 1A, B). In AD brain tissue, the A4-positive variants were observed to be localized together with Aβ plaques, which were identified using the monoclonal antibody against Aβ₁₋₇ (6E10, FIG. 1A). Similar to A4 staining, the C6T-recognized oligomers also co-localized with Aβ aggregates (FIG. 1B). In addition, C6T-positive structures were observed around nuclei (FIG. 1B). In order to further confirm whether C6T-stained Aβ variants within neurons, tissue was double stained with C6T and neuronal marker MAP2. Perinuclear cytoplasmic C6T-immunoreactive inclusions were observed within neurons labeled by MAP2 (FIG. 1C).

### Example 3

### C6T- and A4-Recognized Oligomeric Variants in the Brains of APP/PS1 Mice.

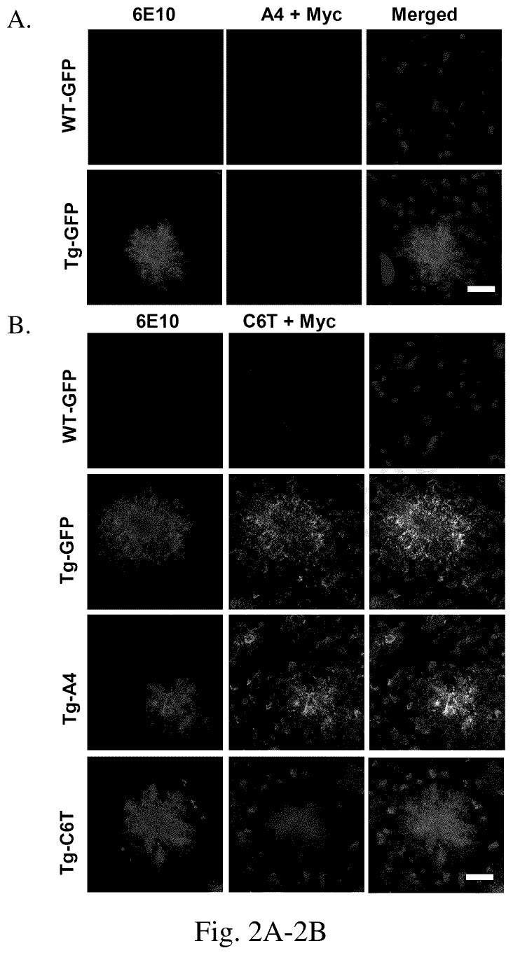

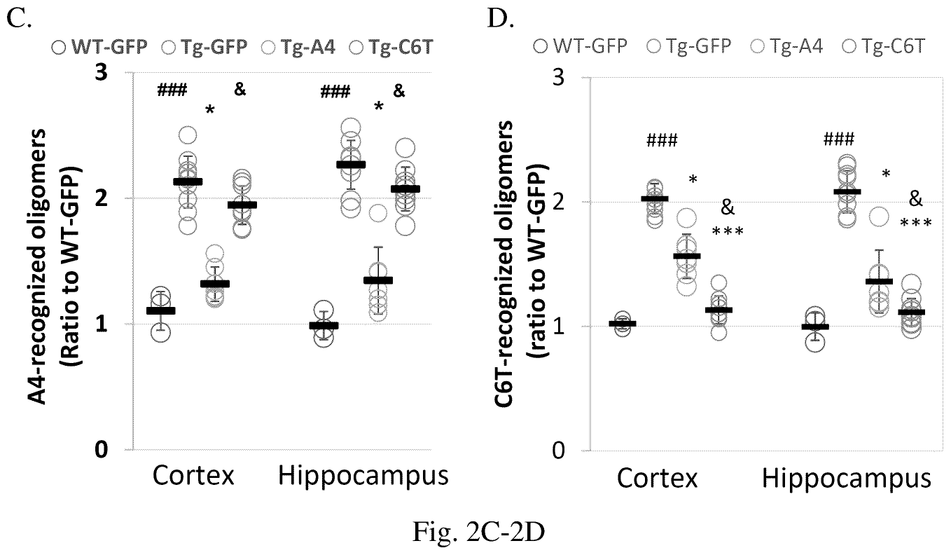

Following staining on human brain sections, similar studies were performed on brain slices of APP/PS1 mice, a mouse model of AD. Expression of the oligomeric Aβ variants recognized by C6T and A4 was not detected in the brains of WT mice (FIG. 2A, B), similar to observations with ND human brain tissue (FIG. 1A, B). While A4 staining was observed in human AD brain tissue, expression of A4-recognized Aβ variants was not observed in the brains of APP/PS1 mice (FIG. 2A). However, similar to the results observed in the human AD brain tissue, co-localization of C6T-positive oligomers was observed with Aβ plaques marked by specific Aβ antibody 6E10 as well as C6T-positive staining inclusions (FIG. 2B). Furthermore, the brain sections of mice treated with the rAAV-C6T showed greatly reduced staining of C6T-immunoreactive structures (FIG. 2B).

The levels of the A4 and C6T recognized oligomeric Aβ variants in mouse brain homogenates were determined by ELISA. The APP/PS1 mice showed significant increases in the levels of both A4- and C6T-specific oligomeric variants (FIG. 2C, D, ^{###}p<0.001), similar to previous observation (He 2019). In the mice treated with either rAAV-C6T or rAAV-A4, the levels of both A4-reactive oligomeric variants (FIG. 2C, *p<0.05). and C6T-reactive variant levels (FIG. 2D, *p<0.05) were lowered compared to the vehicle treated mice.

### Example 4

### Intracellular Localization of C6T-Recognized Oligomeric Variants.

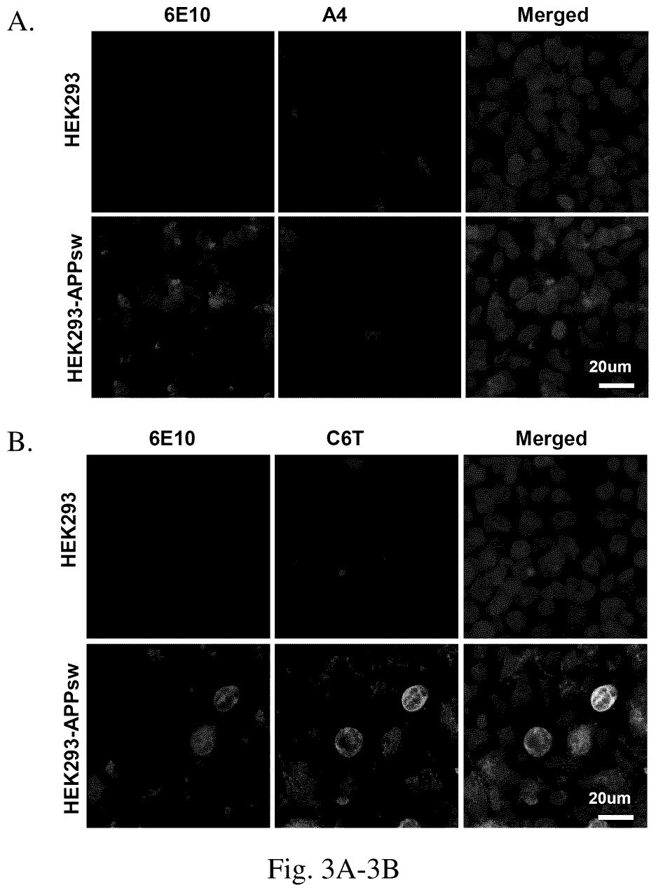

HEK293 cells overexpressing human Aβ were cultured and double immunostained with scFvs and Aβ specific antibody 6E10. Generation of A4- or C6T-recognized Aβ species were not observed in HEK293 cells without transfection of mutated human APP (FIG. 3A, 3B). A4-recognized Aβ variants were not observed in HEK293 cells overexpressing APPswe (FIG. 3A), however, oligomeric Aβ variants recognized by C6T were observed in the cytoplasm of the HEK293 overexpressing mutated human APP (FIG. 3B). The C6T scFv co-stained with Aβ peptide labeled by 6E10 confirmed the presence of oligomeric Aβ. The intracellular localization of C6T recognized Aβ variants is consistent with our observations of human AD and APP/PS1 mouse brain tissue. This result confirms that C6T-recognized oligomeric Aβ variants were intracellularly located in human and APP/PS1 mouse brain tissue (FIG. 1, 2).

### Example 5

### Both C6T and A4 Decrease Aβ Deposits in APP/PS1 Mice

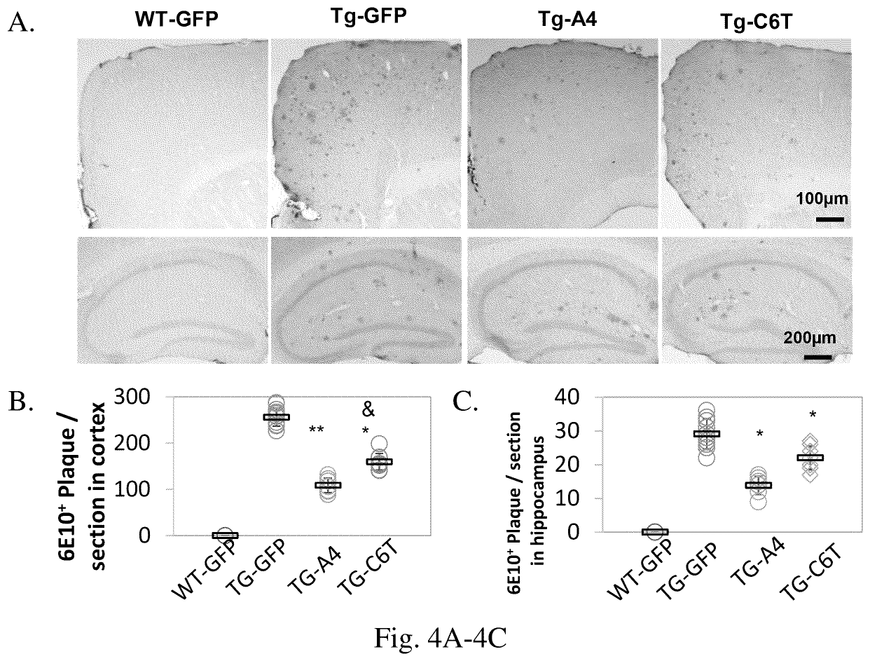

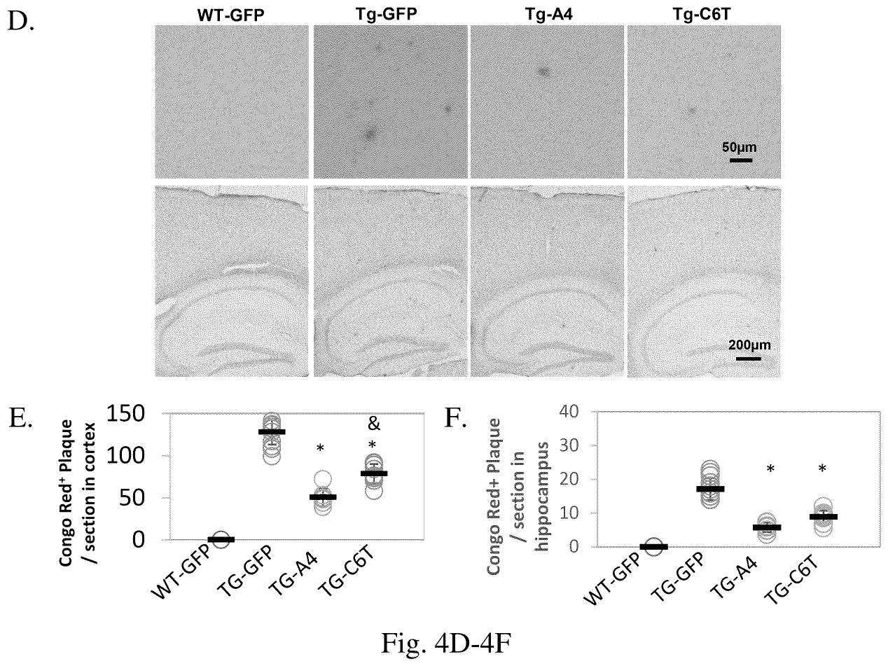

Aβ accumulation develops into plaques in the parenchyma (27). Immunostaining showed amyloid accumulation 6E10-specific in the brains of APP/PS1 mice but not in wild type mice (FIG. 4A). The number of plaques significantly decreased in the cortex (FIG. 4B, **p<0.01, *p<0.05) and the hippocampus (FIG. 4C,*p<0.05) of mice receiving either rAAV-A4 and rAAV-C6T compared to the littermate GFP vehicle group. Interestingly, the number of 6E10-staining plaques in the cortex decreased significantly more in the rAAV-A4 treated mice compared to the rAAV-C6T treated mice (FIG. 4B, p<0.05) and were lower, though not statistically significantly, in the hippocampus as well.

The plaques are considered to be composed of a tangle of regularly ordered amyloid fibrillar aggregates (28). Histological staining using Congo-red dye showed water-insoluble fibrillar plaques in the brains of APP/PS1 mice but not in wild type mice (FIG. 4D). A significant decrease in the number of Congo-red deposits was observed in both the cortex (FIG. 3E, *p<0.05) and hippocampus (FIG. 3F, *p<0.05) in brain tissue of mice treated with rAAV-A4 and rAAV-C6T compared to littermate GFP vehicle treated mice. Similar to the results obtained with the 6E10-labeled plaques (FIG. 3B), a significant decrease in the number of Congo-red structures was observed in the cortex in mice treated with rAAV-A4 compared to rAAV-C6T (FIG. 3F, ^{&}p<0.05) and levels were also lower in the hippocampus.

### Both A4 and C6T Decrease Microgliosis and Gliosis in APP/PS1 Mice.

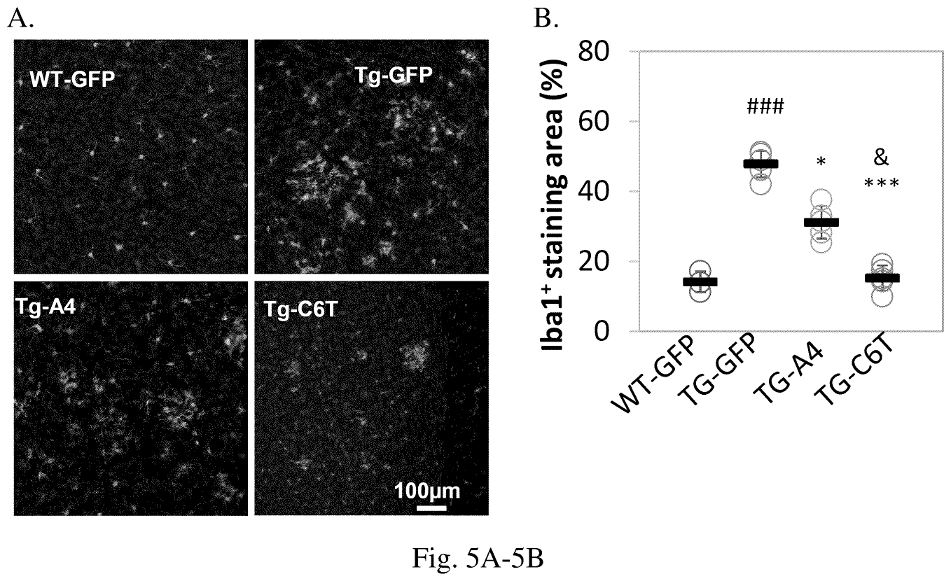

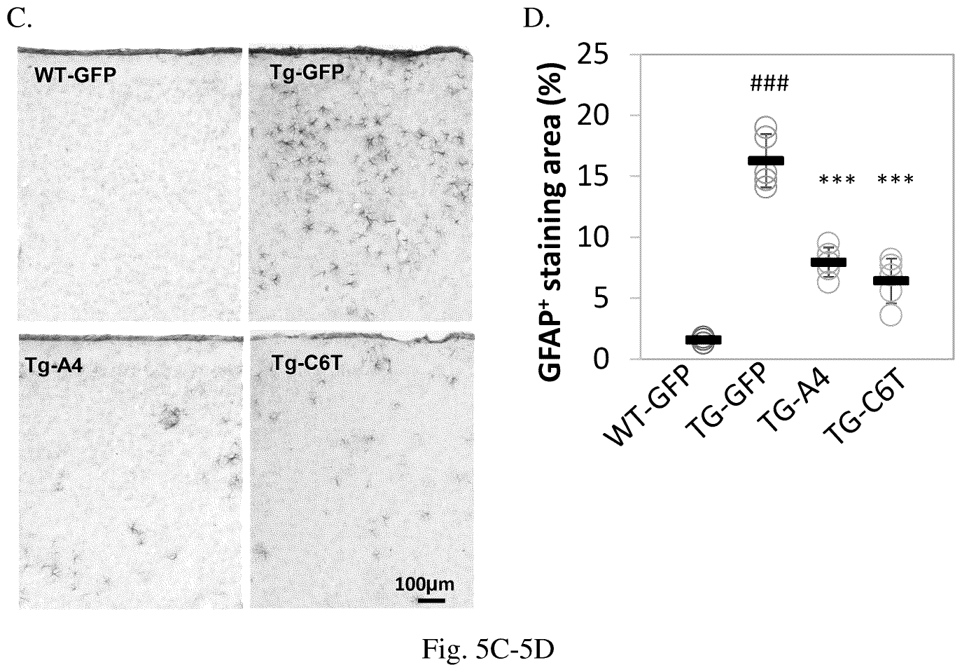

High levels of Aβ burden may be cytotoxic and activate glial cells leading to altered inflammatory responses (27, 29). Since microglia activation is a response to the presence of toxic substrates (27, 29), the effects of C6T and A4 treatment on inflammation can be assessed through microglial activation. Double immunostaining was performed using antibody Iba1, a microglial marker, and 6E10 as an Aβ marker. Representative images showed a correlation of Iba1-positive cells with 6E10-labeling Aβ in the cortex of WT-GFP, Tg-GFP, Tg-A4 and Tg-C6T (FIG. 5A). Compared to age-matched WT-GFP vehicle group, the area of reactive microglia was greatly elevated in Tg-GFP vehicle mice (FIG. 5B, ^{###}p<0.001). In the C6T and A4 treated groups, there was a significant reduction in the area of reactive microglial compared to littermate GFP transgenic vehicle group (FIG. 5B, ***p<0.001, *p<0.05). Interestingly, while mice treated with rAAV-A4 showed reduced plaque formation compared to mice treated with rAAV-C6T, mice treated with rAAV-C6T showed significantly reduced reactive microglia compared to the rAAV-A4 group (FIG. 5B, ^{&}p<0.05).

### Both A4 and C6T Decrease Astrocyte Activation in APP/PS1 Mice.

Glial fibrillary acidic protein (GFAP) is another marker of neurodegeneration and neuronal injury (30). Immunostaining was performed using an antibody against GFAP to label astrocytes and Congo-red as a marker of fibrillar deposits. Representative images showed a correlation of GFAP positive cells with Congo-red positive aggregates in the cortex of WT-GFP, Tg-GFP, Tg-A4 and Tg-C6T (FIG. 4C). Compared to age-matched WT-GFP vehicle group, the area of reactive astrocytes was greatly elevated in Tg-GFP vehicle mice (FIG. 5D, ^{###}p<0.001). In the treated groups, both C6T and A4 significantly reduced the area of reactive astrocytes, compared to littermate GFP transgenic vehicle group (FIG. 5D, ***p<0.001).

### C6T Reversed Dendrite Spine Loss and Synaptic Aggregates.

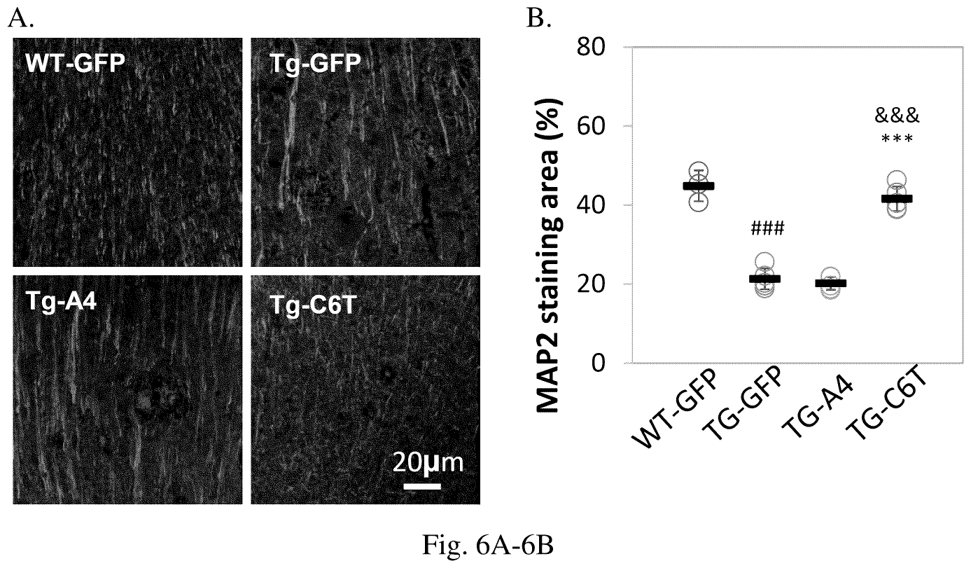

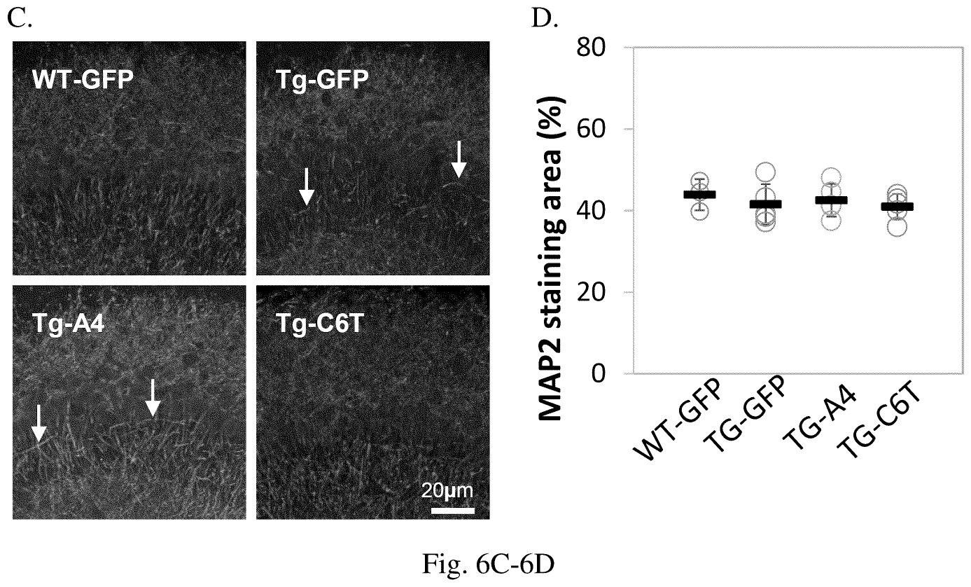

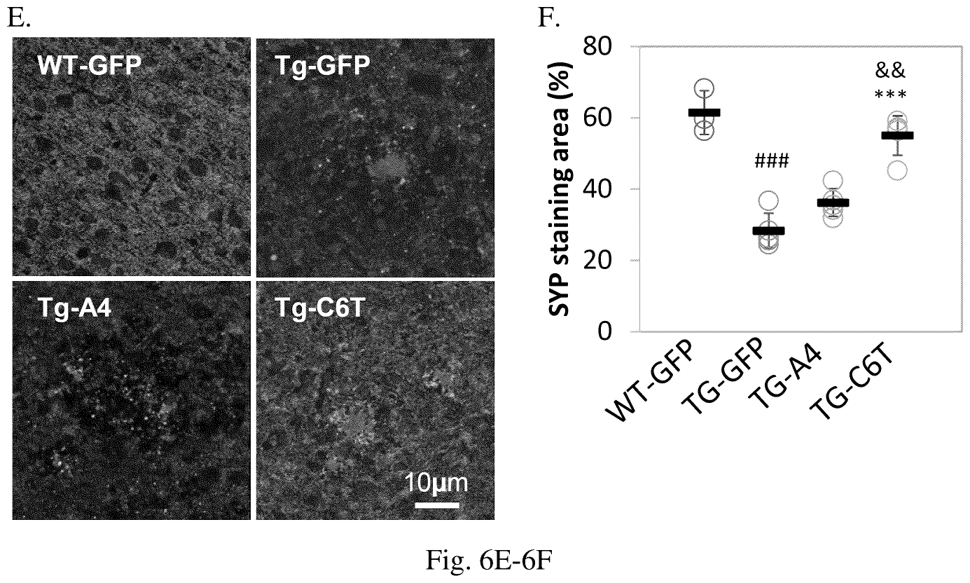

The number of the dendrites of pyramidal neurons decreases in APP based transgenic mouse models of AD (29, 31, 32). Dendritic density and organization were imaged using MAP-2 staining (FIG. 6A). Compared to WT mice, the transgenic GFP treated mice had higher staining of MAP2 and a decreased number of MAP2-positive dendrites and spines (FIG. 6B, ^{###}p<0.001), as reported previously (22). In the experiment, treatment with A4 did not rescue dendritic organization and decreased MAP2 staining, similar to the area of MAP2-positive in littermate Tg-GFP vehicle mice (FIG. 6B, p >0.05). However, treatment with scFv-C6T restored the dendritic appearance and organization to levels similar in the vehicle WT mice (FIG. 6A). The area of MAP2-immunoreactive dendrites was significantly elevated, compared to the transgenic vehicle mice (FIG. 6B, ***p<0.001) and to the mice treated with A4 (FIG. 6B, ^{&&&}p<0.001).

In the CA3 region of the hippocampus, there was also considerable disorganization of the dendrites in littermate transgenic vehicle group as well as the A4-treated mice (FIG. 6C, arrowhead). However, in the C6T treated group, the well-organized structure of dendrites were similar to age-matched WT-GFP mice (FIG. 6C). Quantitatively, MAP2 staining did not show a significant loss of MAP2 expression in the hippocampus among the groups (FIG. 6D, p >0.05). Synaptic changes in the treated mice were also characterized using synaptophysin (SYP, a pre-synaptic marker) and 6E10 labeling Aβ plagues. Strong staining of neurite aggregates close to 6E10-positive plaques were observed in the cortex of transgenic GFP control mice (FIG. 6E). There was a significant decrease in the area of SYP staining in transgenic vehicle mice, compared to wild type mice (FIG. 6F, ^{###}p<0.001). Treatment with A4 did not significantly change the area of SYP-positive aggregates close to Aβ plaques when compared to the TG mice (FIG. 6E, F, p >0.05). However, treatment with C6T significantly decreased SYP aggregates (strong staining) around Aβ plaques to levels observed in the wild-type mice (FIG. 6E) rescuing SYP staining compared to levels observed in the transgenic vehicle treated mice (FIG. 6F, ***p<0.001) and to the mice treated with scFv-A4 (FIG. 6F, ^{&&&}p<0.001).

### C6T Greatly Improves Neurogenesis.

To determine if the decrease in oligomeric Aβ levels affected the rate of neurogenesis, neurogenesis was tracked using doublecortin (DCX), a classical marker for immature neurons (33, 34). Positive staining was observed in the subgranular zone (SGZ) of the dentate gyrus of the hippocampus (FIG. 7A). A significant decrease was found in the number of DCX positive cells of the transgenic vehicle treated mice when compared to similarly treated WT-GFP mice (FIG. 7B, ^{##}p<0.01). With the treatment of rAAV-C6T the number of DCX-positive cells was increased in the SGZ to levels higher than even the WT mice (FIG. 7B, ***p<0.001). Treatment with rAAV-A4 also increased the number of immature neurons compared to littermate Tg vehicle mice (FIG. 7B, *p<0.05) but the levels were significantly lower than the mice receiving rAAV-C6T (FIG. 7B, ^{&&&}p<0.001).

### C6T Rescues Survival in APP/PS1 Mice.

All mice were purchased at an age of one month and housed following standard IAUCC protocols. WT mice receiving rAAV-GFP administration as a vehicle negative group were housed for 9 months with no loss of mice. APP/PS1 mice have a high mortality rate with around 40 percent by the age of 12 months old (35). Similar to these reports, the APP/PS1 mice treated with rAAV-GFP at two months of age as a vehicle control group had a similar mortality rate after 7 months with only 12 mice surviving (FIG. 7C). The rAAV-A4 treated mice delayed mortality, but did not change the survival rate at 9 months. Quite impressively, the mice treated with rAAV-C6T had survival levels similar to wild-type with only 1 mouse dying at the very beginning of treatment (FIG. 7C).

### Example 6

Viral Infection Exclusion from Brains of Mice Receiving rAAV-scFv

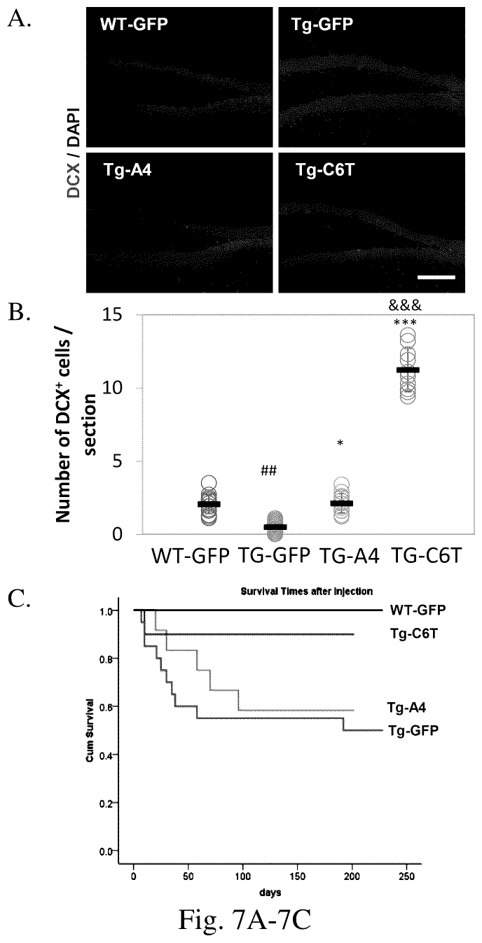

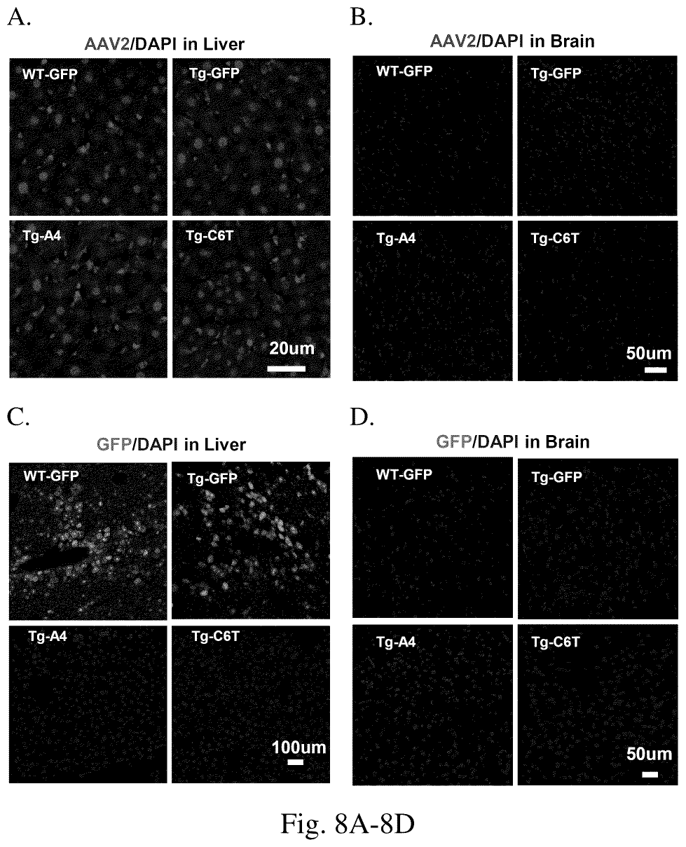

To verify that viral infection and subsequent scFv expression was occurring in hepatic cells and not brain cells, liver and brain tissue samples were analyzed essentially as described previously (He, 2019). While rAAV capsid AAV2 was visualized in all liver tissues of rAAV2/8 vector groups following intraperitoneal (IP) delivery for 9 months (FIG. 8A), no AAV2 staining was observed in brains of the same mice receiving rAAV (FIG. 8B). Next, we observed GFP expression directly under fluorescence confocal microscopy in the liver sections of the mice receiving rAAV-GFP, rAAV-A4 and rAAV-C6T. A strong fluorescent expression of GFP was seen in the hepatic cells receiving rAAV-GFP but not in the results obtained with AAV capsid staining, no GFP expression was observed in brain samples of mice infected with rAAV-GFP, rAAV-A4 or rAAV-C6T (FIG. 8D). These results indicate that active rAAVs expression was taking place in liver cells but not in brain cells.

ScFv Expression in Liver and Brain Tissue after Infection with rAAV

Expression of the scFvs by infected liver cells and transport into the brain by the ApoB tag was verified. The liver tissue sections were immunostained with an antibody against FLAG, a marker for the A4 and C6T scFvs. As expected, FLAG was visualized in the hepatic cells of the APP/PS1 mice administered rAAV-A4 and rAAV-C6T mice but not in that of WT and Tg mice receiving rAAV-GFP (FIG. 9A). Similarly, the immunostaining of brain sections showed a strong labeling of FLAG in the brains of APP/PS1 mice receiving rAAV-A4 and rAAV-C6T but negative staining in the WT and APP/PS1 mice receiving rAAV-GFP (FIG. 9B), indicating that the ApoB tag on the scFvs could facilitate the transport across the BBB. Levels of scFvs in the respective brain tissues were determined by ELISA. A commercial protein containing multiple tag markers including FLAG was utilized as a standard. Brain levels of the FLAG tag were measured and normalized to pg/mg of the brain tissues. ELISA results showed high expression of scFv levels in the cortex tissue of the mice transfected with rAAV-A4 and rAAV-C6T but not in the WT-GFP and Tg-GFP mice (FIG. 9C). These results indicate successful expression of scFv by infected liver cells and transport across the BBB into the brain.

Administration of scFv-A4 and C6T does not Change APP Metabolism in APP/PS1 Mice

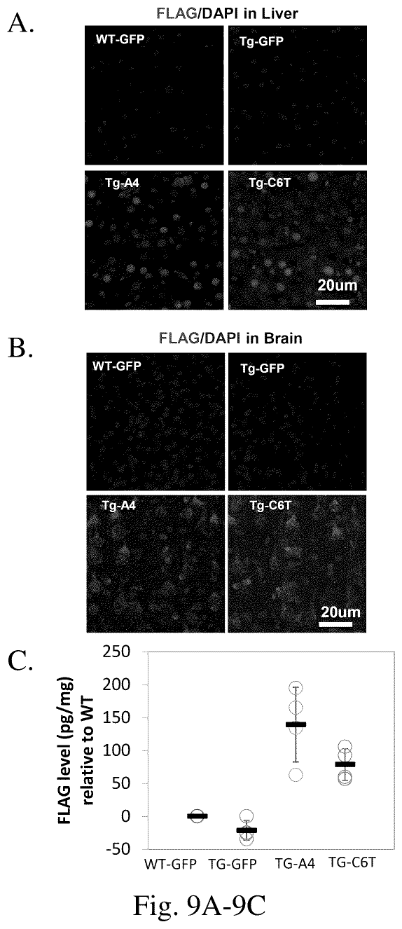

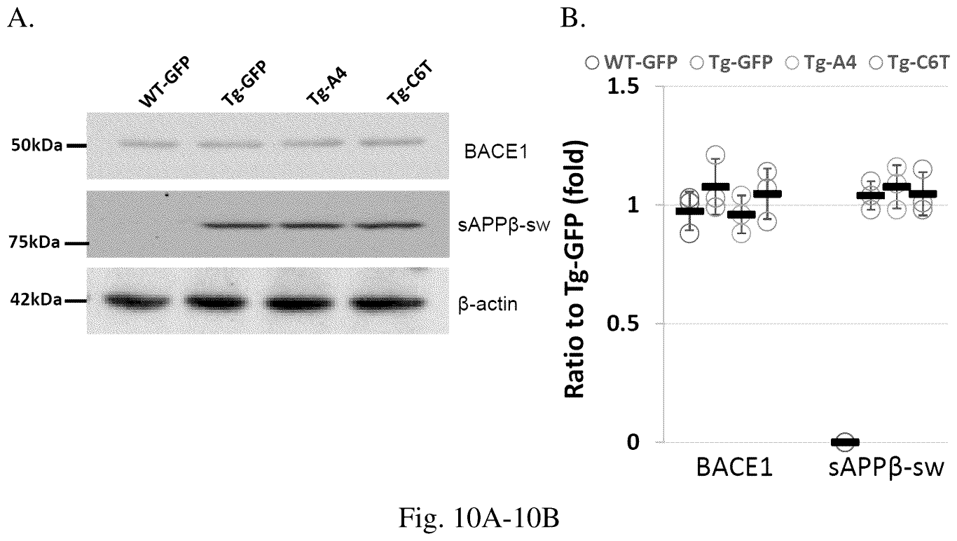

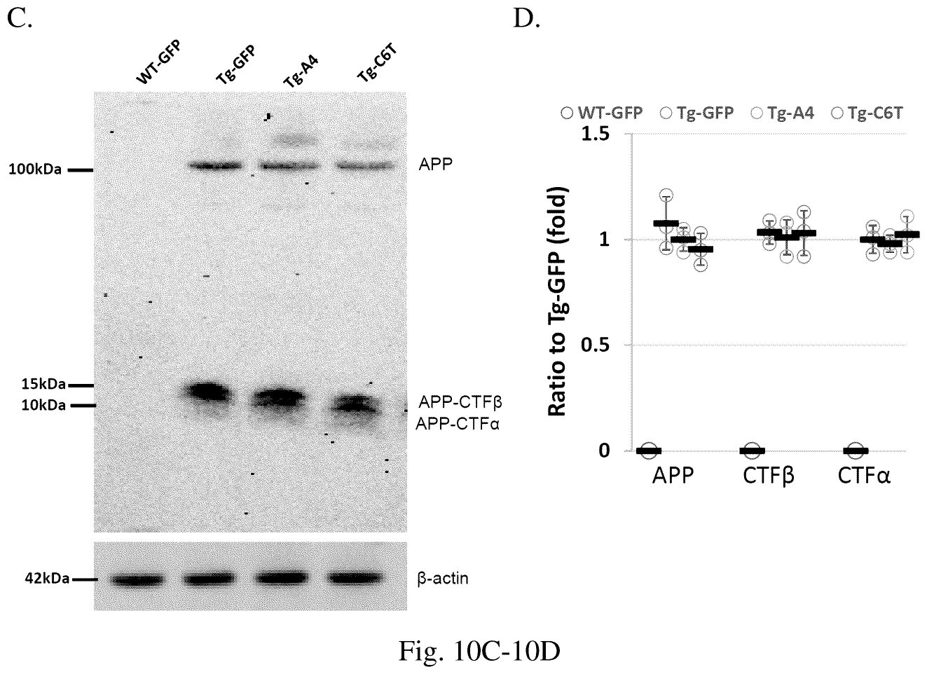

To exclude the possible effects of scFv administration on APP processing, APP cleavage fragments were analyzed by Western blot (FIG. 10). There were not any obvious differences in expression levels of the APP cleavage enzyme BACE1 between WT and transgenic mice with or without scFv application. The cleavage fragment by BACE1, human soluble β APP Swedish mutation (sAPPβ-sw) was expressed in human APP transgenic mice but not WT. The levels of sAPPβ-sw, however, were not significant different in the transgenic mice with or without scFv application (FIG. 10A, B). To further confirm the APP amyloidogenic processing, the cleavage of APP C-terminal fragment was immunoblotted. We did not find any significant expression changes of APP as well as a, 0 fragments of C-terminals (CTFα, and CTFβ) among Tg-GFP, Tg-A4 and Tg-C6T, but no visible expression of APP fragments in WT-GFP (FIG. 10C, D).

### Discussion of Examples 2-6

scFvs A4 and C6T selectively bind two different conformational variants of oligomeric Aβ (Table 3)(17, 18, 21). The scFvs were used to immunostain human post-mortem AD brain tissue, where the A4 scFv stained oligomeric Aβ variants primarily localized around the dense core of amyloid plaques. However, in an APP/PS1 AD mouse model, there was only slight staining of A4-recognized oligomeric Aβ in the diffuse plaques and similarly only slight staining of cells expressing mutant APP transgenes. Since A4 binds an Aβ oligomeric species estimated to be around the size of dodecamers, these results suggest that this population of oligomeric Aβ may be involved in Aβ oligomer-seeded fibril formation, consistent with previous reports indicating the ability of soluble high molecular weight Aβ species to seed Aβ plaque deposition (36,37). In contrast, the C6T scFv which selectively binds a small soluble toxic AD brain derived oligomeric Aβ variant(18,20) stained around plaques in post-mortem human AD brain tissue and APP/PS1 mice. Significantly, intraneuronal staining of C6T recognized oligomeric Aβ was observed in brain sections of both AD samples and APP transgenic mice, as well as in the cultured cells expressing APPswe (FIG. 3). Intraneuronal oligomeric Aβ deposits were previously identified(38) and were postulated to be one of the earliest events in AD pathogenesis (39,40).

The therapeutic effects of selectively targeting oligomeric Aβ species bound by A4 and C6T scFvs in vivo using a mouse model of AD were investigated. Since the A4 scFv binds an oligomeric Aβ species that can be generated in vitro and is located extracellularly (FIG. 1) and C6T binds an oligomeric Aβ species that is generated in vivo and is located intracellularly (FIG. 1C), the results indicate the therapeutic benefit of targeting intracellular or extracellular toxic Aβ species. The ApoB sequence that binds to the LDL receptor was added to the N-terminal region of the A4 and C6T scFvs to facilitate transport across the brain blood barrier (BBB) in vivo (23, 41, 42). The scFv constructs were expressed in hepatic cells in an APP/PS1 AD mouse model by viral infection using a recombinant human adeno-associated virus (rAAV) as a vector as described previously (22). The tagged scFvs are expressed by the infected hepatic cells, secreted into the blood and then cross the BBB into the brain. Both C6T and A4 scFvs were efficiently expressed as measured by ELISA (FIG. 8) and high levels of scFvs were detected in brain tissue indicating successful transport across the BBB essentially similar to what was reported previously (FIG. 9) (22). While both A4 and C6T scFvs were present in the mouse brain tissue, the therapeutic benefit of the two treatments differed. Loss of dendritic spine density and organization, including loss of synaptic buttons, are common pathological features of APP/PS1 mice (29, 31, 32). Treatment with C6T restored MAP2-labeled dendrite spine density to wild-type levels, while treatment with A4 did not alter spine density compared to vehicle treated mice (FIG. 5A, B). Treatment with C6T also restored synaptic density and organization including clustering of SYP-labeled synaptic buttons again to wild-type levels, while treatment with A4 did not significantly improve synaptic density or organization compared to the vehicle treated mice (FIG. 5E, F). Treatment with C6T also elevated neurogenesis in the hippocampus of APP/PS1 mice compared to that treatment with A4 (FIG. 6A, B). Most dramatically, the high mortality rate of the APP/PS1 mice was rescued in the C6T treated mice again restoring wild type levels, while mortality was slightly delayed but essentially unchanged from the control group in the A4 treated mice (FIG. 6C).

Inflammation in the brain correlates strongly with neurodegeneration in AD(43) where microgliosis and astrogliosis are common features associated with Aβ deposition(44,45). While treatment with both C6T and A4 reduced astrogliosis (FIG. 4), treatment with C6T reduced microglial activation significantly better than treatment with A4, restoring inflammation to levels similar to that in the wild type group (FIG. 4B). These results indicate that selectively targeting C6T recognized oligomeric Aβ provides substantially greater therapeutic benefit towards mortality, neuronal integrity and reducing inflammation compared to targeting A4 recognized oligomeric Aβ. However, when we analyze how the scFvs influence amyloid plaque levels the results are quite different. Amyloid plaque levels as determined by 6E10 staining (FIG. 3A-C) and by Congo-red staining (FIG. 3D-F) showed significant reductions in the A4 treated mice, but only modest reduction in the C6T treated mice. Treatment with A4 lowered both C6T and A4 recognized oligomeric Aβ levels in the cortex and hippocampus, while treatment with C6T lowered C6T recognized Aβ variant levels to essentially wild-type levels but did not alter A4 recognized Aβ variant levels. The reduction in C6T levels may also be a result of virally produced C6T sterically blocking binding of the C6T scFv in the immunostains. Since treatment with C6T did not reduce plaque levels as effectively as treatment with A4, the results suggest a correlation of certain oligomeric Aβ variants (such as those recognized by A4) with the formation of Aβ plaques (36, 37, 46), while the smaller intracellularly brain derived C6T-recognized oligomeric Aβ species play a more important role in synaptic toxicity.

These results indicate differential effects that various oligomeric Aβ species can have on neuronal health and function. Extracellularly generated oligomeric Aβ species such as those recognized by the A4 scFv seem to play an important role in seeding fibril formation and inducing astrogliosis, while intracellularly generated oligomeric Aβ species such as those recognized by the C6T scFv are highly toxic to synapses, as previously suggested for low molecular weight Aβ oligomers (47,48). There are multiple different mechanisms by which C6T may decrease levels of oligomeric Aβ and provide a therapeutic benefit. The antigen-antibody complex could be degraded by proteolysis or cleared by activated microglia in the brains, the complex may sterically interfere blocking potential toxic interactions of oligomeric Aβ, C6T may redirect the Aβ aggregation pathway, or some combination of these effects.

### Exemplary Sequences and Constructs

Table 5 provides amino acid sequences and encoding nucleotide sequences of three scFv antibodies of the invention (C6T, A4, and E1) and portions thereof, including complementarity determining regions as demarkated by Kabat and Chothia. scFv sequences include (GGGGS)₃ linkers between V_{H} and V_{L} domains. Certain sequences may include N-terminal amino acids (e.g. initial Met-Ala in SEQ ID NOS:24, 25, 47, 48, 64) that arose from initial construction and expression of scFv proteins that need not be included in fusion proteins or expression products of the invention. Certain sequences may include signal peptides that are normally cleaved upon expression and would not be present in expression products of the invention. Certain sequences include C-terminal (e.g. His and/or Myc tag) sequences that facilitate analysis or protein purification which need not be present in products of the invention. Exemplary AAV constructs (e.g., SEQ ID NOS:81-84 comprise IgGκ signal peptides and blood brain barrier (BBB) tags and FLAG tags.

TABLE 5
Exemplary Constructs and Sequences

| SEQ | | |
|---|---|---|
| ID | | |
| NO | | |
| 1 | C6T-VH-CDR1 | agctatgcca tgagc |
| 2 | C6T-VH-CDR1 | SYAMS |
| 3 | C6T-VH-CDR2 | gctattagtg gtagtggtgg tagcacatac tacgcagact ccgtgaaggg |
| | | c |
| 4 | C6T-VH-CDR2 | AISGSGGSTY YADSVKG |
| 5 | C6T-VH-CDR3 | agctatggtt cagttaaaat aagctgcttt gactac |
| 6 | C6T-VH-CDR3 | SYGSVKISCF DY |
| 7 | C6T-VL-CDR1 | aagtccagcc agagtgttct ttacaactcc aacaataaga actacttagc |
| | | t |
| 8 | C6T-VL-CDR1 | KSSQSVLYNS NNKNYLA |
| 9 | C6T-VL-CDR2 | tgggcatcaa cccgggaatc c |
| 10 | C6T-VL-CDR2 | WASTRES |
| 11 | C6T-VL-CDR3 | cagcaatttt atagtactcc tccgact |
| 12 | C6T-VL-CDR3 | QQFYSTPPT |
| 13 | C6T-VH-CDR1 | agtggtagtg gtggtagc |
| 14 | C6T-VH-CDR1 | GFTFSSY |
| 15 | C6T-VH-CDR2 | tgggcatca |
| 16 | C6T-VH-CDR2 | SGSGGS |
| 17 | C6T-VL-CDR1 | agccagagtg ttctttacaa ctccaacaat aagaactac |
| 18 | C6T-VL-CDR1 | SQSVLYNSNN KNY |
| 19 | C6T-VL-CDR2 | tgggcatca |
| 20 | C6T-VL-CDR2 | WAS |
| 21 | C6T-VL-CDR3 | ttttatagta ctcctccg |
| 22 | C6T-VL-CDR3 | FYSTPP |
| 23 | ScFv-C6T | atggcccagg tacagctgca ggagtctggg ggaggcttgg tacagcctgg |
| | | ggggtccctg agactctcct gtgcagcctc tggattcacc tttagcagct |
| | | atgccatgag ctgggtccgc caggctccag ggaaggggct ggagtgggtc |
| | | tcagctatta gtggtagtgg tggtagcaca tactacgcag actccgtgaa |
| | | gggccgattc accatctcca gagacaattc caagaacacg ctgtatctgc |
| | | aaatgaacag cctgagagct gaggacacgg ctgtgtatta ctgtgcgaag |
| | | agctatggtt cagttaaaat aagctgcttt gactactggg gccagagcac |
| | | cctggtcacc gtctcctcag gtggaggcgg ttcaggcgga ggtggctctg |
| | | gcggtggcgg atcggaaatt gtgctgacgc agtctccaga ctccctggct |
| | | gtgtctctgg gcgagagggc caccatcaac tgcaagtcca gccagagtgt |
| | | tctttacaac tccaacaata agaactactt agcttggtac cagcagaaac |
| | | caggacagtc tcctgagttg ctcatttact gggcatcaac ccgggaatcc |
| | | ggggtccctg accgattcag tggcagcggg tctgggacag aattcactct |
| | | taccatcagc agcctgcagg ctgaggatgt ggcagtttat tactgtcagc |
| | | aattttatag tactcctccg acttttggcc aggggaccaa gctggagatc |
| | | aaacgt |
| 24 | SCFv-C6T | MAQVQLQESG GGLVQPGGSL RLSCAASGFT FSSYAMSWVR QAPGKGLEWV |
| | | SAISGSGGST YYADSVKGRF TISRDNSKNT LYLQMNSLRA EDTAVYYCAK |
| | | SYGSVKISCF DYWGQSTLVT VSSGGGGSGG GGSGGGGSEI VLTQSPDSLA |
| | | VSLGERATIN CKSSQSVLYN SNNKNYLAWY QQKPGQSPEL LIYWASTRES |
| | | GVPDRFSGSG SGTEFTLTIS SLQAEDVAVY YCQQFYSTPP TFGQGTKLEI |
| | | KR |
| 25 | ScFv-C6T | MAQVQLQESG GGLVQPGGSL RLSCAASGFT FSSYAMSWVR QAPGKGLEWV |
| | | SAISGSGGST YYADSVKGRF TISRDNSKNT LYLQMNSLRA EDTAVYYCAK |
| | | SYGSVKISCF DYWGQSTLVT VSSGGGGSGG GGSGGGGSEI VLTQSPDSLA |
| | | VSLGERATIN CKSSQSVLYN SNNKNYLAWY QQKPGQSPEL LIYWASTRES |
| | | GVPDRFSGSG SGTEFTLTIS SLQAEDVAVY YCQQFYSTPP TFGQGTKLEI |
| | | KRAAAHHHHH HGAAEQKLIS EEDLNGAA |
| 26 | A4-VH-CDR1 | agctatccca tgagc |
| 27 | A4-VH-CDR1 | SYPMS |
| 28 | A4-VH-CDR2 | gcgattcagc atactggtgc gccgacaact tacgcagact ccgtgaaggg |
| | | c |
| 29 | A4-VH-CDR2 | AIQHTGAPTT YADSVKG |
| 30 | A4-VH-CDR3 | gcgtttccgc cgtttgacta c |
| 31 | A4-VH-CDR3 | AFPPFDY |
| 32 | A4-VL-CDR1 | cgggcaagtc agagcattag cagctattta aat |
| 33 | A4-VL-CDR1 | RASQSISSYL N |
| 34 | A4-VL-CDR2 | tctgcatcct ctttgcaaag t |
| 35 | A4-VL-CDR2 | SASSLQS |
| 36 | A4-VL-CDR3 | caacagcggg agactgggcc taaggcg |
| 37 | A4-VL-CDR3 | QQRETGPKA |
| 38 | A4-VH-CDR2 | cagcatactg gtgcgccg |
| 39 | A4-VH-CDR2 | QHTGAP |
| 40 | A4-VL-CDR1 | agtcagagca ttagcagcta t |
| 41 | A4-VL-CDR1 | SQSISSY |
| 42 | A4-VL-CDR2 | tctgcatcc |
| 43 | A4-VL-CDR2 | SAS |
| 44 | A4-VL-CDR3 | cgggagactg ggcctaag |
| 45 | A4-VL-CDR3 | RETGPK |
| 46 | SCFv-A4 | atggccgagg tgcagctgtt ggagtctggg ggaggcttgg tacagcctgg |
| | | ggggtccctg agactctcct gtgcagcctc tggattcacc tttagcagct |
| | | atcccatgag ctgggtccgc caggctccag ggaaggggct ggagtgggtc |
| | | tcagcgattc agcatactgg tgcgccgaca acttacgcag actccgtgaa |
| | | gggccggttc accatctcca gagacaattc caagaacacg ctgtatctgc |
| | | aaatgaacag cctgagagcc gaggacacgg ccgtatatta ctgtgcgaaa |
| | | gcgtttccgc cgtttgacta ctggggccag ggaaccctgg tcaccgtctc |
| | | gagcggtgga ggcggttcag gcggaggtgg cagcggcggt ggcgggtcga |
| | | cggacatcca gatgacccag tctccatcct ccctgtctgc atctgtagga |
| | | gacagagtca ccatcacttg ccgggcaagt cagagcatta gcagctattt |
| | | aaattggtat cagcagaaac cagggaaagc ccctaagctc ctgatctatt |
| | | ctgcatcctc tttgcaaagt ggggtcccat caaggttcag tggcagtgga |
| | | tctgggacag atttcactct caccatcagc agtctgcaac ctgaagattt |
| | | tgcaacttac tactgtcaac agcgggagac tgggcctaag gcgttcggcc |
| | | aagggaccaa ggtggaaatc aaacgg |
| 47 | ScFv-A4 | MAEVQLLESG GGLVQPGGSL RLSCAASGFT FSSYPMSWVR QAPGKGLEWV |
| | | SAIQHTGAPT TYADSVKGRF TISRDNSKNT LYLQMNSLRA EDTAVYYCAK |
| | | AFPPFDYWGQ GTLVTVSSGG GGSGGGGSGG GGSTDIQMTQ SPSSLSASVG |
| | | DRVTITCRAS QSISSYLNWY QQKPGKAPKL LIYSASSLQS GVPSRFSGSG |
| | | SGIDFTLTIS SLQPEDFATY YCQQRETGPK AFGQGTKVEI KR |
| 48 | ScFv-A4 | MAEVQLLESG GGLVQPGGSL RLSCAASGFT FSSYPMSWVR QAPGKGLEWV |
| | | SAIQHTGAPT TYADSVKGRF TISRDNSKNT LYLQMNSLRA EDTAVYYCAK |
| | | AFPPFDYWGQ GTLVTVSSGG GGSGGGGSGG GGSTDIQMTQ SPSSLSASVG |
| | | DRVTITCRAS QSISSYLNWY QQKPGKAPKL LIYSASSLQS GVPSRFSGSG |
| | | SGTDFTLTIS SLQPEDFATY YCQQRETGPK AFGQGTKVEI KRAAAHHHHH |
| | | HGAAEQKLIS EEDLNGAA |
| 49 | E1-VH-CDR2 | tctattcagc ctgagggtag gcggacagcg tacgtagact ccgtgaaggg |
| | | c |
| 50 | E1-VH-CDR2 | SIQPEGRRTA YVDSVK |
| 51 | E1-VH-CDR3 | ccgccggaga ggtttgacta c |
| 52 | E1-VH-CDR3 | PPERFDY |
| 53 | E1-VL-CDR2 | gctgcatcca gtttgcaaag t |
| 54 | E1-VL-CDR2 | AASSLQS |
| 55 | E1-VL-CDR3 | gaacagagtt acagtacccc taatacg |
| 56 | E1-VL-CDR3 | QQSYSTPNT |
| 57 | E1-VH-CDR2 | cagcctgagg gtaggcgg |
| 58 | E1-VH-CDR2 | QPEGRR |
| 59 | E1-VL-CDR2 | gctgcatcc |
| 60 | E1-VL-CDR2 | AAS |
| 61 | E1-VL-CDR3 | agttacagta cccctaat |
| 62 | E1-VL-CDR3 | SYSTPN |
| 63 | ScFv-E1 | atggccgagg tgcagctgtt ggagtctggg ggaggcttgg tacagcctgg |
| | | ggggtccctg agactctcct gtgcagcctc tggattcacc tttagcagct |
| | | atgccatgag ctgggtccgc caggctccag ggaaggggct ggagtgggtc |
| | | tcatctattc agcctgaggg taggcggaca gcgtacgtag actccgtgaa |
| | | gggccggttc accatctcca gagacaattc caagaacacg ctgtatctac |
| | | aaatgaacag cctgagagcc gaggacacgg ccgtatatta ctgtgcgaaa |
| | | ccgccggaga ggtttgacta ctggggccag ggaaccctgg tcaccgtctc |
| | | gagcggtgga ggcggttcag gcggaggtgg cagcggcggt ggcgggtcga |
| | | cggacatcca gatgacccag tctccatcct ccctgtctgc atctgtagga |
| | | gacagagtca ccatcacttg ccgggcaagt cagagcatta gcagctattt |
| | | aaattggtat cagcagaaac cagggaaagc ccctaagctc ctgatctatg |
| | | ctgcatccag tttgcaaagt ggggtcccat caaggttcag tggcagtgga |
| | | tctgggacag atttcactct caccatcagc agtctgcaac ctgaagattt |
| | | tgcaacttac tactgtcaac agagttacag tacccctaat acgttcggcc |
| | | aagggaccaa ggtggaaatc aaacgg |
| 64 | ScFv-E1 | MAEVQLLESG GGLVQPGGSL RLSCAASGFT FSSYAMSWVR QAPGKGLEWV |
| | | SSIQPEGRRT AYVDSVKGRF TISRDNSKNT LYLQMNSLRA EDTAVYYCAK |
| | | PPERFDYWGQ GTLVTVSSGG GGSGGGGSGG GGSTDIQMTQ SPSSLSASVG |
| | | DRVTITCRAS QSISSYLNWY QQKPGKAPKL LIYAASSLQS GVPSRFSGSG |
| | | SGTDFTLTIS SLQPEDFATY YCQQSYSTPN TFGQGTKVEI KR |
| 65 | ApoB BBB tag | tcatctgtca ttgatgcact ccaatacaaa ttagagggca ccacaagatt |
| | | gacaagaaag agaggattga agttagccac agctctgtct ctgagcaaca |
| | | aatttgtgga gggtagt |
| 66 | ApoB BBB tag | SSVIDALQYK LEGTTRLTRK RGLKLATALS LSNKFVEGS |
| 67 | LDLR-binding | HPWCCGLRLD LR |
| | peptide | |
| 68 | LDLR-binding | YHFNGCEDPL CR |
| | peptide | |
| 69 | penetratin | RQIKIWFQNR RMKWKK |
| 70 | SecPen BBB | cagagcctgg cccaggaact gggcctgaac gaacgacaga tcaaaatctg |
| | tag | gttccagaac cgccgcatga agtggaaaaa a |
| 71 | SecPen BBB | QSLAQELGLN ERQIKIWFQN RRMKWKK |
| | tag | |
| 72 | TAT | YGRKKRRQRR R |
| | transduction | |
| | peptide | |
| 73 | TAT | GRKKRRQRRR PPQ |
| | transduction | |
| | peptide | |
| 74 | angiopep1 | TFFYGGCRGK RNNFKTEEY |
| 75 | angiopep2 | TFFYGGSRGK RNNFKTEEY |
| 76 | transportan | CLIKKALAAL AKLNIKLLYG ASNLTWG |
| 77 | TER1-binding | GGHKAKGPRK LGS |
| | peptide B6 | |
| 78 | TER1-binding | THRPPMWSPV WP |
| | peptide THR | |
| 79 | TER1-binding | HAIYPRH |
| | peptide | |
| 80 | signal | METDTLLLWV LLLWVPGSTG |
| | peptide | |
| | (IgGK) | |
| 81 | AAV-C6T | gtcgacgccg ccacaatgga tggagacaga cacactcctg ctatgggtac |
| | Kozak-IgK- | tgctgctctg ggttccaggt tccactggtg acgagctctc atctgtcatt |
| | SacI-ApoB- | gatgcactcc aatacaaatt agagggcacc acaagattga caagaaagag |
| | NcoI-ScFv- | aggattgaag ttagccacag ctctgtctct gagcaacaaa tttgtggagg |
| | NotI-FLAG- | gtagtggtgg aggcggatct gccatggccc aggtacagct gcaggagtct |
| | Stop | gggggaggct tggtacagcc tggggggtcc ctgagactct cctgtgcagc |
| | | ctctggattc acctttagca gctatgccat gagctgggtc cgccaggctc |
| | | cagggaaggg gctggagtgg gtctcagcta ttagtggtag tggtggtagc |
| | | acatactacg cagactccgt gaagggccga ttcaccatct ccagagacaa |
| | | ttccaagaac acgctgtatc tgcaaatgaa cagcctgaga gctgaggaca |
| | | cggctgtgta ttactgtgcg aagagctatg gttcagttaa aataagctgc |
| | | tttgactact ggggccagag caccctggtc accgtctcct caggtggagg |
| | | cggttcaggc ggaggtggct ctggcggtgg cggatcggaa attgtgctga |
| | | cgcagtctcc agactccctg gctgtgtctc tgggcgagag ggccaccatc |
| | | aactgcaagt ccagccagag tgttctttac aactccaaca ataagaacta |
| | | cttagcttgg taccagcaga aaccaggaca gtctcctgag ttgctcattt |
| | | actgggcatc aacccgggaa tccggggtcc ctgaccgatt cagtggcagc |
| | | gggtctggga cagaattcac tcttaccatc agcagcctgc aggctgagga |
| | | tgtggcagtt tattactgtc agcaatttta tagtactcct ccgacttttg |
| | | gccaggggac caagctggag atcaaacgtg cggccgcaga ttacaaggat |
| | | gacgatgaca agtga |
| 82 | AAV-C6T | METDTLLLWV LLLWVPGSTG DELSSVIDAL QYKLEGTTRL TRKRGLKLAT |
| | IgK-ApoB- | ALSLSNKFVE GSGGGGSAMA QVQLQESGGG LVQPGGSLRL SCAASGFTFS |
| | SCFV-FLAG | SYAMSWVRQA PGKGLEWVSA ISGSGGSTYY ADSVKGRFTI SRDNSKNTLY |
| | | LQMNSLRAED TAVYYCAKSY GSVKISCFDY WGQSTLVTVS SGGGGSGGGG |
| | | SGGGGSEIVL TQSPDSLAVS LGERATINCK SSQSVLYNSN NKNYLAWYQQ |
| | | KPGQSPELLI YWASTRESGV PDRFSGSGSG TEFTLTISSL QAEDVAVYYC |
| | | QQFYSTPPTF GQGTKLEIKR AAADYKDDDD K |
| 83 | AAV-A4 | gccgccacaa tggatggaga cagacacact cctgctatgg gtactgctgc |
| | Kozak-IgK- | tctgggttcc aggttccact ggtgacgagc tctcatctgt cattgatgca |
| | SacI-ApoB- | ctccaataca aattagaggg caccacaaga ttgacaagaa agagaggatt |
| | NcoI-ScFv- | gaagttagcc acagctctgt ctctgagcaa caaatttgtg gagggtagtg |
| | NotI-FLAG- | gtggaggcgg atctgccatg gccgaggtgc agctgttgga gtctggggga |
| | Stop | ggcttggtac agcctggggg gtccctgaga ctctcctgtg cagcctctgg |
| | | attcaccttt agcagctatc ccatgagctg ggtccgccag gctccaggga |
| | | aggggctgga gtgggtctca gcgattcagc atactggtgc gccgacaact |
| | | tacgcagact ccgtgaaggg ccggttcacc atctccagag acaattccaa |
| | | gaacacgctg tatctgcaaa tgaacagcct gagagccgag gacacggccg |
| | | tatattactg tgcgaaagcg tttccgccgt ttgactactg gggccaggga |
| | | accctggtca ccgtctcgag cggtggaggc ggttcaggcg gaggtggcag |
| | | cggcggtggc gggtcgacgg acatccagat gacccagtct ccatcctccc |
| | | tgtctgcatc tgtaggagac agagtcacca tcacttgccg ggcaagtcag |
| | | agcattagca gctatttaaa ttggtatcag cagaaaccag ggaaagcccc |
| | | taagctcctg atctattctg catcctcttt gcaaagtggg gtcccatcaa |
| | | ggttcagtgg cagtggatct gggacagatt tcactctcac catcagcagt |
| | | ctgcaacctg aagattttgc aacttactac tgtcaacagc gggagactgg |
| | | cagattacaa ttcggccaag ggaccaaggt ggaaatcaaa cgggcggccg |
| | | gcctaaggcg |
| 84 | AAV-A4 | METDTLLLWV LLLWVPGSTG DELSSVIDAL QYKLEGTTRL TRKRGLKLAT |
| | IgK-ApoB- | ALSLSNKFVE GSGGGGSAMA EVQLLESGGG LVQPGGSLRL SCAASGFTFS |
| | SCFV-FLAG | SYPMSWVRQA PGKGLEWVSA IQHTGAPTTY ADSVKGRFTI SRDNSKNTLY |
| | | LQMNSLRAED TAVYYCAKAF PPFDYWGQGT LVTVSSGGGG SGGGGSGGGG |
| | | STDIQMTQSP SSLSASVGDR VTITCRASQS ISSYLNWYQQ KPGKAPKLLI |
| | | YSASSLQSGV PSRFSGSGSG TDFTLTISSL QPEDFATYYC QQRETGPAFG |
| | | QGTKVEIKRA AADYKDDDDK |
| 85 | ApoB-C6TscFv | SSVIDALQYK LEGTTRLTRK RGLKLATALS LSNKFVEGSG GGGSAMAQVQ |
| | | LQESGGGLVQ PGGSLRLSCA ASGFTFSSYA MSWVRQAPGK GLEWVSAISG |
| | | SGGSTYYADS VKGRFTISRD NSKNTLYLQM NSLRAEDTAV YYCAKSYGSV |
| | | KISCFDYWGQ STLVTVSSGG GGSGGGGSGG GGSEIVLTQS PDSLAVSLGE |
| | | RATINCKSSQ SVLYNSNNKN YLAWYQQKPG QSPELLIYWA STRESGVPDR |
| | | FSGSGSGTEF TLTISSLQAE DVAVYYCQQF YSTPPTFGQG TKLEIKR |
| 86 | ApoB-A4scFv | SSVIDALQYK LEGTTRLTRK RGLKLATALS LSNKFVEGSG GGGSAMAEVQ |
| | | LLESGGGLVQ PGGSLRLSCA ASGFTFSSYP MSWVRQAPGK GLEWVSAIQH |
| | | TGAPTTYADS VKGRFTISRD NSKNTLYLQM NSLRAEDTAV YYCAKAFPPF |
| | | DYWGQGTLVT VSSGGGGSGG GGSGGGGSTD IQMTQSPSSL SASVGDRVTI |
| | | TCRASQSISS YLNWYQQKPG KAPKLLIYSA SSLQSGVPSR FSGSGSGTDF |
| | | TLTISSLQPE DFATYYCQQR ETGPAFGQGT KVEIKR |
| 87 | ApoB-E4scFv | SSVIDALQYK LEGTTRLTRK RGLKLATALS LSNKFVEGSG GGGSAMAEVQ |
| | | LLESGGGLVQ PGGSLRLSCA ASGFTFSSYA MSWVRQAPGK GLEWVSSIQP |
| | | EGRRTAYVDS VKGRFTISRD NSKNTLYLQM NSLRAEDTAV YYCAKPPERF |
| | | DYWGQGTLVT VSSGGGGSGG GGSGGGGSTD IQMTQSPSSL SASVGDRVTI |
| | | TCRASQSISS YLNWYQQKPG KAPKLLIYAA SSLQSGVPSR FSGSGSGTDF |
| | | TLTISSLQPE DFATYYCQQS YSTPNTFGQG TKVEIKR |
| 107 | C6TscFv | QVQLQESGGG LVQPGGSLRL SCAASGFTFS SYAMSWVRQA PGKGLEWVSA |
| | | ISGSGGSTYY ADSVKGRFTI SRDNSKNTLY LQMNSLRAED TAVYYCAKSY |
| | | GSVKISCFDY WGQSTLVTVS SGGGGSGGGG SGGGGSEIVL TQSPDSLAVS |
| | | LGERATINCK SSQSVLYNSN NKNYLAWYQQ KPGQSPELLI YWASTRESGV |
| | | PDRFSGSGSG TEFTLTISSL QAEDVAVYYC QQFYSTPPTF GQGTKLEIKR |
| 108 | A4scFv | EVQLLESGGG LVQPGGSLRL SCAASGFTFS SYPMSWVRQA PGKGLEWVSA |
| | | IQHTGAPTTY ADSVKGRFTI SRDNSKNTLY LQMNSLRAED TAVYYCAKAF |
| | | PPFDYWGQGT LVTVSSGGGG SGGGGSGGGG STDIQMTQSP SSLSASVGDR |
| | | VTITCRASQS ISSYLNWYQQ KPGKAPKLLI YSASSLQSGV PSRFSGSGSG |
| | | TDFTLTISSL QPEDFATYYC QQRETGPAFG QGTKVEIKR |
| 109 | E4scFv | EVQLLESGGG LVQPGGSLRL SCAASGFTFS SYAMSWVRQA PGKGLEWVSS |
| | | IQPEGRRTAY VDSVKGRFTI SRDNSKNTLY LQMNSLRAED TAVYYCAKPP |
| | | ERFDYWGQGT LVTVSSGGGG SGGGGSGGGG STDIQMTQSP SSLSASVGDR |
| | | VTITCRASQS ISSYLNWYQQ KPGKAPKLLI YAASSLQSGV PSRFSGSGSG |
| | | TDFTLTISSL QPEDFATYYC QQSYSTPNTF GQGTKVEIKR |

### Example 6

### Disruption of Neuronal Proteostasis by Intracellular Oligomeric Aβ in Human AD Brain.

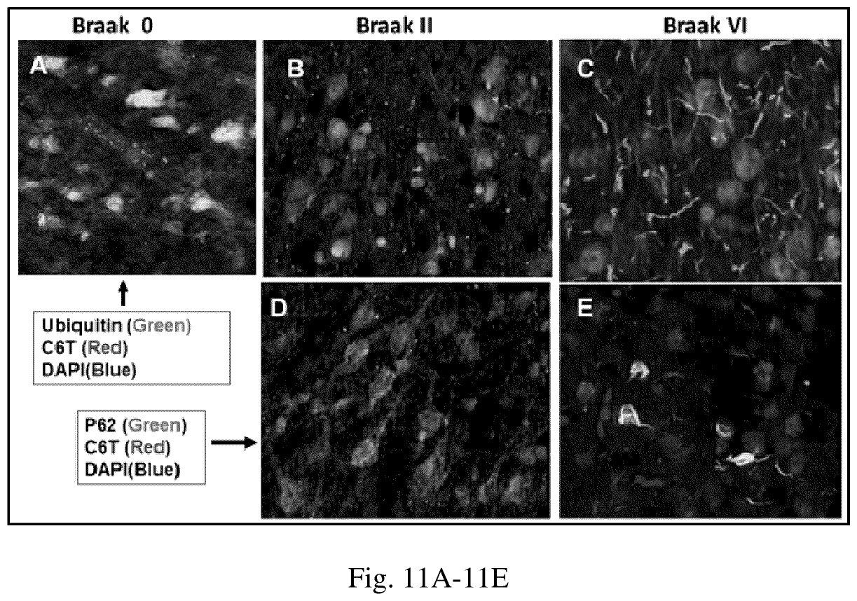

The connection between intracellularly generated oligomeric Aβ aggregates bound by C6T and cell proteostasis by immunohistochemical (IHC) analysis of post-mortem human brain tissue was demonstrated. Brain tissue from post-mortem human Braak Stage 0, II and VI cases was stained to visualize co-localization of C6T with two different markers of cell proteostasis: ubiquitin and P62 a marker for autophagy (FIG. 11). The Braak stage 0 case shows some limited cytoplasmic C6T staining (red) which always co-localizes with robust ubiquitin staining (green) indicating healthy neurons with a functional ubiquitin proteasome system (UPS) (FIG. 11A). The Braak stage II case shows the presence of both healthy neurons with high ubiquitin levels and little if any C6T staining, as well as stressed neurons showing a disrupted UPS that have extensive C6T staining and little if any ubiquitin staining (FIG. 11B) and extensive P62 staining (green) (FIG. 11D). The Braak stage VI case shows much less C6T staining with very little cytoplasmic ubiquitin staining, but significant axonal staining (FIG. 11C) and very limited P62 staining that colocalized with C6T staining (FIG. 11E) indicating a severely dysfunctional UPS. These images show the progressive loss of proteostasis in neurons, first affecting the cytoplasm of neurons containing C6T reactive oligomeric Aβ aggregates, and later affecting the neuronal processes.

Restoration of Proteostasis with C6T Treatment.

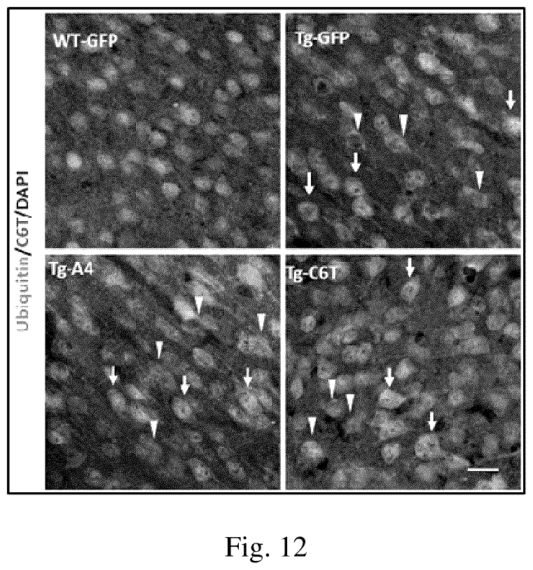

Direct therapeutic benefit that treatment with C6T has on cell proteostasis was demonstrated in the AD mouse model. Brain tissue from WT and APP/PS1 mice treated with either C6T, A4 or sham (GFP) (ref 26) was stained to visualize co-localization of C6T and ubiquitin (FIG. 12). The WT mice have substantial ubiquitin staining but no C6T staining indicating a properly functioning UPS. The sham (GFP) treated TG mice have significant C6T staining in neuronal cytoplasm comprising two different staining patterns: 1) healthy neurons with high ubiquitin levels and little if any C6T staining (full arrows in FIG. 12), and 2) neurons with a disrupted UPS that have high levels of C6T staining and little if any ubiquitin staining (arrowheads in FIG. 12). The A4 treated mice have essentially the same staining pattern as that observed in the sham treated mice. In contrast, the C6T treated mice closely resemble the WT mice with restoration of ubiquitin staining, where the reduced C6T staining mainly co-localizes with ubiquitin (full arrows in FIG. 12). Interestingly the sham and A4 treated mice show basically similar staining patterns to the Braak stage II human brain tissue with significant disruption of the UPS while the C6T treated mice show a similar staining pattern to the Braak stage 0 human brain tissue with a functional UPS. These results provide convincing evidence that targeting and clearing a toxic intracellularly generated Aβ variant restores the UPS and neuronal proteostasis, while targeting extracellular oligomeric Aβ decreased plaque loads, but did not restore the UPS or improve neuronal health, mirroring the sham treated mice. These results demonstrate that C6T provides a new therapeutic approach to treating AD to restore neuronal proteostasis in human brain.

The invention is further described by the following numbered paragraphs:

1. A method of reducing or inhibiting Aβ toxicity in a subject, or a method of treating, ameliorating, or preventing an Aβ disease or disorder, or a method of slowing cognitive decline in a patient diagnosed with an Aβ disease or disorder, or a method of reducing amyloid plaque load in a subject, or a method of preventing memory loss or cognitive decline in an asymptomatic patient, which comprises administering to the subject or expressing in the subject an effective amount of an antibody that selectively binds to oligomeric Aβ, wherein the antibody is engineered for effective transport across the blood brain barrier (BBB).

2. The method of paragraph 1, wherein the antibody reduces intracellular Aβ inclusions.

3. The method of paragraph 1, wherein the antibody reduces or inhibits amyloid accumulation, fibrillary deposits, microgliosis, astrogliosis, and/or dendritic spine loss.

4. The method of paragraph 1, wherein administering the antibody to a subject increases neurogenesis.

5. The method of any one of paragraphs 1 to 4, wherein the antibody comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence AISGSGGSTYYADSVKG (SEQ ID NO:4); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence KSSQSVLYNSNNKNYLA (SEQ ID NO:8); (e) a light chain complementarity determining region 2 (VL-CDR2) comprising the amino acid sequence WASTRES (SEQ ID NO:10); and (f) a light chain complementarity determining region 3 (VL-CDR3) comprising the amino acid sequence QQFYSTPPT (SEQ ID NO:12); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence SGSGGS (SEQ ID NO:16); (c′) a heavy chain complementarity determining region 3 (VH-CDR3) comprising the amino acid sequence SYGSVKISCFDY (SEQ ID NO:6); (d′) a light chain complementarity determining region 1 (VL-CDR1) comprising the amino acid sequence SQSVLYNSNNKNY (SEQ ID NO:18); (e′) a light chain complementarity determining region 2 (VL-CDR2) comprising the amino acid sequence WAS (SEQ ID NO:20); and (f′) a light chain complementarity determining region 3 (VL-CDR3) comprising the amino acid sequence FYSTPP (SEQ ID NO:22).

6. The method of paragraph 5, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:24.

7. The method of any one of paragraphs 1 to 4, wherein the antibody comprises: (a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QHTGAP (SEQ ID NO:39); (c′) a heavy chain complementarity determining region 3 (VH-CDR3) comprising the amino acid sequence AFPPFDY (SEQ ID NO:31); (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence SAS (SEQ ID NO:43); and (f′) a light chain complementarity determining region 3 (VL-CDR3) comprising the amino acid sequence RETGPK (SEQ ID NO:45).

8. The method of paragraph 7, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:47.

9. The method of any one of paragraphs 1 to 4, wherein the antibody comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence SIQPEGRRTAYVDSVK (SEQ ID NO:50); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence AASSLQS (SEQ ID NO:54); and (f) a light chain complementarity determining region 3 (VL-CDR3) comprising the amino acid sequence QQSYSTPNT (SEQ ID NO:56); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QPEGRR (SEQ ID NO:58); (c′) a heavy chain complementarity determining region 3 (VH-CDR3) comprising the amino acid sequence PPERFDY (SEQ ID NO:52; (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence AAS (SEQ ID NO:60); and (f′) a light chain complementarity determining region 3 (VL-CDR3) comprising the amino acid sequence SYSTPN (SEQ ID NO:62).

10. The method of paragraph 9, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:64.

11. The method of any one of paragraphs 1 to 10, wherein the antibody comprises a scFv, a humanized antibody, a fully human antibody, a chimeric antibody, a single-chain antibody, a diabody, an intrabody, or antigen-binding fragments thereof.

12. The method of any one of paragraphs 1 to 10, wherein the antibody comprises or is linked to a polypeptide which binds to a blood brain barrier (BBB) receptor.

13. The method of paragraph 12, wherein the BBB receptor comprises low-density lipoprotein (LDL) receptor (LDLR), transferrin receptor, or insulin-like growth factor receptor.

14. The method of paragraph 12 or 13, wherein the polypeptide which binds to a BBB comprises an LDLR-binding domain of apoB.

15. The method of any one of paragraphs 1 to 10, wherein the antibody comprises or is linked to a cell penetrating peptide (CPP).

16. An antibody engineered for transport across the blood brain barrier (BBB) which comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence AISGSGGSTYYADSVKG (SEQ ID NO:4); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence KSSQSVLYNSNNKNYLA (SEQ ID NO:8); (e) a light chain complementarity determining region 2 (VL-CDR2) comprising the amino acid sequence WASTRES (SEQ ID NO:10); and (f) a light chain complementarity determining region 3 (VL-CDR3) comprising the amino acid sequence QQFYSTPPT (SEQ ID NO:12); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence SGSGGS (SEQ ID NO:16); (c′) a heavy chain complementarity determining region 3 (VH-CDR3) comprising the amino acid sequence SYGSVKISCFDY (SEQ ID NO:6); (d′) a light chain complementarity determining region 1 (VL-CDR1) comprising the amino acid sequence SQSVLYNSNNKNY (SEQ ID NO:18); (e′) a light chain complementarity determining region 2 (VL-CDR2) comprising the amino acid sequence WAS (SEQ ID NO:20); and (f′) a light chain complementarity determining region 3 (VL-CDR3) comprising the amino acid sequence FYSTPP (SEQ ID NO:22).

17. An antibody engineered for transport across the blood brain barrier (BBB) which comprises: (a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QHTGAP (SEQ ID NO:39); (c′) a heavy chain complementarity determining region 3 (VH-CDR3) comprising the amino acid sequence AFPPFDY (SEQ ID NO:31); (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence SAS (SEQ ID NO:43); and (f′) a light chain complementarity determining region 3 (VL-CDR3) comprising the amino acid sequence RETGPK (SEQ ID NO:45).

18. An antibody engineered for transport across the blood brain barrier (BBB) which comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence SIQPEGRRTAYVDSVK (SEQ ID NO:50); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence AASSLQS (SEQ ID NO:54); and (f) a light chain complementarity determining region 3 (VL-CDR3) comprising the amino acid sequence QQSYSTPNT (SEQ ID NO:56); or wherein the antibody comprises: (a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QPEGRR (SEQ ID NO:58); (c′) a heavy chain complementarity determining region 3 (VH-CDR3) comprising the amino acid sequence PPERFDY (SEQ ID NO:52; (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence AAS (SEQ ID NO:60); and (f′) a light chain complementarity determining region 3 (VL-CDR3) comprising the amino acid sequence SYSTPN (SEQ ID NO:62).

19. The antibody of any one of paragraphs 16 to 18 which comprises or is linked to a polypeptide which binds to a blood brain barrier (BBB) receptor.

20. The antibody of paragraph 19, wherein the BBB receptor comprises low-density lipoprotein (LDL) receptor (LDLR), transferrin receptor, or insulin-like growth factor receptor.

21. The antibody of paragraph 19, wherein the polypeptide which binds to a BBB comprises an LDLR-binding domain of apoB.

22. The antibody of paragraph 19, which comprises SEQ ID NO:85, SE ID NO:86, or SEQ ID NO:87.

23. The antibody of any one of paragraphs 16 to 18, wherein the antibody comprises or is linked to a cell penetrating peptide (CPP).

24. A nucleic acid which encodes the antibody of any of paragraphs 16 to 23.

25. The nucleic acid of paragraph 24, which comprises SEQ ID NO:81 or SEQ IDNO:83.

26. The nucleic acid of paragraph 24 or 25, which comprises DNA, RNA, nucleotide analogs or combinations thereof.

27. An expression vector which comprises or expresses the nucleic acid of any one of paragraphs 24 to 26.

28. A pharmaceutical composition which comprises the antibody of any one of paragraphs 16 to 23, or the nucleic acid of any one of paragraphs 24 to 26, or the expression vector of paragraph 27.

29. A method of determining whether a candidate agent is useful for reducing or inhibiting Aβ toxicity, or for treating, ameliorating, or preventing an Aβ disease or disorder, or for reducing amyloid plaque load in a subject, or for preventing memory loss or cognitive decline in an asymptomatic patient, which comprises comparing the candidate agent to a control reagent, wherein the control reagent comprises or expresses an antibody engineered for transport across the blood brain barrier (BBB) which comprises: (a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence AISGSGGSTYYADSVKG (SEQ ID NO:4); (c) a heavy chain complementarity determining region 3 (VH-CDR3) comprising the amino acid sequence SYGSVKISCFDY (SEQ ID NO:6); (d) a light chain complementarity determining region 1 (VL-CDR1) comprising the amino acid sequence KSSQSVLYNSNNKNYLA (SEQ ID NO:8); (e) a light chain complementarity determining region 2 (VL-CDR2) comprising the amino acid sequence WASTRES (SEQ ID NO:10); and (f) a light chain complementarity determining region 3 (VL-CDR3) comprising the amino acid sequence QQFYSTPPT (SEQ ID NO:12); or
(a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence SGSGGS (SEQ ID NO:16); (c′) a heavy chain complementarity determining region 3 (VH-CDR3) comprising the amino acid sequence SYGSVKISCFDY (SEQ ID NO:6); (d′) a light chain complementarity determining region 1 (VL-CDR1) comprising the amino acid sequence SQSVLYNSNNKNY (SEQ ID NO:18); (e′) a light chain complementarity determining region 2 (VL-CDR2) comprising the amino acid sequence WAS (SEQ ID NO:20); and (f′) a light chain complementarity determining region 3 (VL-CDR3) comprising the amino acid sequence FYSTPP (SEQ ID NO:22); or
(a) a heavy chain complementarity determining region 1 (VH-CDR1) comprising the amino acid sequence SYPMS (SEQ ID NO:27); (b) a heavy chain complementarity determining region 2 (VH-CDR2) comprising the amino acid sequence AIQHTGAPTTYADSVKG (SEQ ID NO:29); (c) a heavy chain complementarity determining region 3 (VH-CDR3) comprising the amino acid sequence AFPPFDY (SEQ ID NO:31); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence SASSLQS (SEQ ID NO:35); and (f) a light chain complementarity determining region 3 (VL-CDR3) comprising the amino acid sequence QQRETGPKA (SEQ ID NO:37); or
(a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QHTGAP (SEQ ID NO:39); (c′) a heavy chain complementarity determining region 3 (VH-CDR3) comprising the amino acid sequence AFPPFDY (SEQ ID NO:31); (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence SAS (SEQ ID NO:43); and (f′) a light chain complementarity determining region 3 (VL-CDR3) comprising the amino acid sequence RETGPK (SEQ ID NO:45); or
(a) a heavy chain complementarity determining region 1 (VH-CDR1) comprising the amino acid sequence SYAMS (SEQ ID NO:2); (b) a heavy chain complementarity determining region 2 (VH-CDR2) comprising the amino acid sequence SIQPEGRRTAYVDSVK (SEQ ID NO:50); (c) a heavy chain complementarity determining region 3 (VH-CDR3) comprising the amino acid sequence PPERFDY (SEQ ID NO:52); (d) a light chain complementarity determining region 1 (VL-CDR1) comprising the amino acid sequence RASQSISSYLN (SEQ ID NO:33); (e) a light chain complementarity determining region 2 (VL-CDR2) comprising the amino acid sequence AASSLQS (SEQ ID NO:54); and (f) a light chain complementarity determining region 3 (VL-CDR3) comprising the amino acid sequence QQSYSTPNT (SEQ ID NO:56); or
(a′) a heavy chain complementarity determining region 1 (VH-CDR1) comprising the amino acid sequence GFTFSSY (SEQ ID NO:14); (b′) a heavy chain complementarity determining region 2 (VH-CDR2) comprising the amino acid sequence QPEGRR (SEQ ID NO:58); (c′) a heavy chain complementarity determining region 3 (VH-CDR3) comprising the amino acid sequence PPERFDY (SEQ ID NO:52; (d′) a light chain complementarity determining region 1 (VL-CDR1) comprising the amino acid sequence SQSISSY (SEQ ID NO:41); (e′) a light chain complementarity determining region 2 (VL-CDR2) comprising the amino acid sequence AAS (SEQ ID NO:60); and (f′) a light chain complementarity determining region 3 (VL-CDR3) comprising the amino acid sequence SYSTPN (SEQ ID NO:62).

30. The method of paragraph 29, wherein the Aβ disease is Alzheimer's disease.

### REFERENCES

1. Hamley, I. W. (2012) The amyloid beta peptide: a chemist's perspective. Role in Alzheimer's and fibrillization. Chem Rev 112, 5147-5192.
2. Sadigh-Eteghad, S., Sabermarouf, B., Majdi, A., Talebi, M., Farhoudi, M., and Mahmoudi, J. (2015) Amyloid-beta: a crucial factor in Alzheimer's disease. Med Princ Pract 24, 1-10
3. Roher, A. E., Chaney, M. O., Kuo, Y. M., Webster, S. D., Stine, W. B., Haverkamp, L. J., Woods, A. S., Cotter, R. J., Tuohy, J. M., Krafft, G. A., Bonnell, B. S., and Emmerling, M. R. (1996) Morphology and toxicity of Abeta-(1-42) dimer derived from neuritic and vascular amyloid deposits of Alzheimer's disease. J Biol Chem 271, 20631-20635
4. Haass, C., and Selkoe, D. J. (2007) Soluble protein oligomers in neurodegeneration: lessons from the Alzheimer's amyloid beta-peptide. Nat Rev Mol Cell Biol 8, 101-112
5. Shankar, G. M., Li, S., Mehta, T. H., Garcia-Munoz, A., Shepardson, N. E., Smith, I., Brett, F. M., Farrell, M. A., Rowan, M. J., Lemere, C. A., Regan, C. M., Walsh, D. M., Sabatini, B. L., and Selkoe, D. J. (2008) Amyloid-beta protein dimers isolated directly from Alzheimer's brains impair synaptic plasticity and memory. Nat Med 14, 837-842
6. Zhao, L. N., Long, H., Mu, Y., and Chew, L. Y. (2012) The toxicity of amyloid beta oligomers. Int J Mol Sci 13, 7303-7327
7. Ghiso, J., and Frangione, B. (2002) Amyloidosis and Alzheimer's disease. Adv Drug Deliv Rev 54, 1539-1551
8. Selkoe, D. J. (2001) Clearing the brain's amyloid cobwebs. Neuron 32, 177-180
9. Kayed, R., Head, E., Thompson, J. L., McIntire, T. M., Milton, S. C., Cotman, C. W., and Glabe, C. G. (2003) Common structure of soluble amyloid oligomers implies common mechanism of pathogenesis. Science 300, 486-489
10. Gandy, S., Simon, A. J., Steele, J. W., Lublin, A. L., Lah, J. J., Walker, L. C., Levey, A. I., Krafft, G. A., Levy, E., Checler, F., Glabe, C., Bilker, W. B., Abel, T., Schmeidler, J., and Ehrlich, M. E. (2010) Days to criterion as an indicator of toxicity associated with human Alzheimer amyloid-beta oligomers. Ann Neurol 68, 220-230
11. Cizas, P., Budvytyte, R., Morkuniene, R., Moldovan, R., Broccio, M., Losche, M., Niaura, G., Valincius, G., and Borutaite, V. (2010) Size-dependent neurotoxicity of beta-amyloid oligomers. Arch Biochem Biophys 496, 84-92
12. Dahlgren, K. N., Manelli, A. M., Stine, W. B., Jr., Baker, L. K., Krafft, G. A., and LaDu, M. J. (2002) Oligomeric and fibrillar species of amyloid-beta peptides differentially affect neuronal viability. J Biol Chem 277, 32046-32053
13. Stine, W. B., Jr., Dahlgren, K. N., Krafft, G. A., and LaDu, M. J. (2003) In vitro characterization of conditions for amyloid-beta peptide oligomerization and fibrillogenesis. J Biol Chem 278, 11612-11622
14. Bartolini, M., Naldi, M., Fiori, J., Valle, F., Biscarini, F., Nicolau, D. V., and Andrisano, V. (2011) Kinetic characterization of amyloidbeta 1-42 aggregation with a multimethodological approach. Anal Biochem 414, 215-225
15. Ahmed, M., Davis, J., Aucoin, D., Sato, T., Ahuja, S., Aimoto, S., Elliott, J. I., Van Nostrand, W. E., and Smith, S. O. (2010) Structural conversion of neurotoxic amyloidbeta(1-42) oligomers to fibrils. Nat Struct Mol Biol 17, 561-567
16. Kasturirangan, S., Li, L., Emadi, S., Boddapati, S., Schulz, P., and Sierks, M. R. (2012) Nanobody specific for oligomeric beta-amyloid stabilizes nontoxic form. Neurobiol Aging 33, 1320-1328
17. Zameer, A., Kasturirangan, S., Emadi, S., Nimmagadda, S. V., and Sierks, M. R. (2008) Antioligomeric Abeta single-chain variable domain antibody blocks Abeta-induced toxicity against human neuroblastoma cells. J Mol Biol 384, 917-928
18. Kasturirangan, S., Reasoner, T., Schulz, P., Boddapati, S., Emadi, S., Valla, J., and Sierks, M. R. (2013) Isolation and characterization of antibody fragments selective for specific protein morphologies from nanogram antigen samples. Biotechnol Prog 29, 463-471
19. Mrdenovic, D., Majewska, M., I, S. P., Bernatowicz, P., Nowakowski, R., Kutner, W., Lipkowski, J., and Pieta, P. (2019) Size-dependent interaction of amyloid beta oligomers with brain total lipid extract bilayer—fibrillation vs. membrane destruction. Langmuir: the ACS journal of surfaces and colloids
20. Walsh, D. M., and Selkoe, D. J. (2007) A beta oligomers—a decade of discovery. J Neurochem 101, 1172-1184
21. Boddapati, S., Levites, Y., Suryadi, V., Kasturirangan, S., and Sierks, M. R. (2012) Bispecific tandem single chain antibody simultaneously inhibits beta-secretase and promotes alpha-secretase processing of AbetaPP. J Alzheimers Dis 28, 961-969
22. He, P., Xin, W., Schulz, P., and Sierks, M. R. (2019) Bispecific Antibody Fragment Targeting APP and Inducing alpha-Site Cleavage Restores Neuronal Health in an Alzheimer's Mouse Model. Mol Neurobiol 56, 7420-7432
23. Spencer, B., Emadi, S., Desplats, P., Eleuteri, S., Michael, S., Kosberg, K., Shen, J., Rockenstein, E., Patrick, C., Adame, A., Gonzalez, T., Sierks, M., and Masliah, E. (2014) ESCRT-mediated uptake and degradation of brain-targeted alphasynuclein single chain antibody attenuates neuronal degeneration in vivo. Mol Ther 22, 1753-1767
24. Spencer, B. J., and Verma, I. M. (2007) Targeted delivery of proteins across the blood-brain barrier. Proc Natl Acad Sci USA 104, 7594-7599
25. Williams, S. M., Schulz, P., and Sierks, M. R. (2016) Oligomeric alpha-synuclein and beta-amyloid variants as potential biomarkers for Parkinson's and Alzheimer's diseases. Eur J Neurosci 43, 3-16
26. Williams, S. M., Schulz, P., Rosenberry, T. L., Caselli, R. J., and Sierks, M. R. (2017) Blood-Based Oligomeric and Other Protein Variant Biomarkers to Facilitate Pre-Symptomatic Diagnosis and Staging of Alzheimer's Disease. J Alzheimers Dis 58, 23-35
27. Jankowsky, J. L., Fadale, D. J., Anderson, J., Xu, G. M., Gonzales, V., Jenkins, N. A., Copeland, N. G., Lee, M. K., Younkin, L. H., Wagner, S. L., Younkin, S. G., and Borchelt, D. R. (2004) Mutant presenilins specifically elevate the levels of the 42 residue beta-amyloid peptide in vivo: evidence for augmentation of a 42-specific gamma secretase. Hum Mol Genet 13, 159-170
28. Parker, M. H. R., A. B. (2000) Assembly of β-Amyloid Aggregates at the Molecular Level. Chemtracts—Organic Chemistry 13, 51-56
29. Radde, R., Bolmont, T., Kaeser, S. A., Coomaraswamy, J., Lindau, D., Stoltze, L., Calhoun, M. E., Jaggi, F., Wolburg, H., Gengler, S., Haass, C., Ghetti, B., Czech, C., Holscher, C., Mathews, P. M., and Jucker, M. (2006) Abeta42-driven cerebral amyloidosis in transgenic mice reveals early and robust pathology. EMBO Rep 7, 940-946
30. Darlington, D., Li, S., Hou, H., Habib, A., Tian, J., Gao, Y., Ehrhart, J., Sanberg, P. R., Sawmiller, D., Giunta, B., Mori, T., and Tan, J. (2015) Human umbilical cord blood-derived monocytes improve cognitive deficits and reduce amyloid-beta pathology in PSAPP mice. Cell Transplant 24, 2237-2250
31. Bittner, T., Burgold, S., Dorostkar, M. M., Fuhrmann, M., Wegenast-Braun, B. M., Schmidt, B., Kretzschmar, H., and Herms, J. (2012) Amyloid plaque formation precedes dendritic spine loss. Acta Neuropathol 124, 797-807
32. Liebscher, S., Page, R. M., Kafer, K., Winkler, E., Quinn, K., Goldbach, E., Brigham, E. F., Quincy, D., Basi, G. S., Schenk, D. B., Steiner, H., Bonhoeffer, T., Haass, C., Meyer-Luehmann, M., and Hubener, M. (2014) Chronic gamma-secretase inhibition reduces amyloid plaque-associated instability of pre- and postsynaptic structures. Mol Psychiatry 19, 937-946
33. He, P., Staufenbiel, M., Li, R., and Shen, Y. (2014) Deficiency of patched 1-induced Glil signal transduction results in astrogenesis in Swedish mutated APP transgenic mice. Hum Mol Genet 23, 6512-6527
34. He, P., and Shen, Y. (2009) Interruption of beta-catenin signaling reduces neurogenesis in Alzheimer's disease. J Neurosci 29, 6545-6557
35. Gimbel, D. A., Nygaard, H. B., Coffey, E. E., Gunther, E. C., Lauren, J., Gimbel, Z. A., and Strittmatter, S. M. (2010) Memory impairment in transgenic Alzheimer mice requires cellular prion protein. J Neurosci 30, 6367-6374
36. Economou, N. J., Giammona, M. J., Do, T. D., Zheng, X., Teplow, D. B., Buratto, S. K., and Bowers, M. T. (2016) Amyloid beta-Protein Assembly and Alzheimer's Disease: Dodecamers of Abeta42, but Not of Abeta40, Seed Fibril Formation. J Am Chem Soc 138, 1772-1775
37. Walker, L. C., and Jucker, M. (2015) Neurodegenerative diseases: expanding the prion concept. Annu Rev Neurosci 38, 87-103
38. Kulic, L., McAfoose, J., Welt, T., Tackenberg, C., Spani, C., Wirth, F., Finder, V., Konietzko, U., Giese, M., Eckert, A., Noriaki, K., Shimizu, T., Murakami, K., Jrie, K., Rasool, S., Glabe, C., Hock, C., and Nitsch, R. M. (2012) Early accumulation of intracellular fibrillar oligomers and late congophilic amyloid angiopathy in mice expressing the Osaka intra-Abeta APP mutation. Transl Psychiatry 2, e183
39. Takahashi, R. H., Almeida, C. G., Kearney, P. F., Yu, F., Lin, M. T., Milner, T. A., and Gouras, G. K. (2004) Oligomerization of Alzheimer's beta-amyloid within processes and synapses of cultured neurons and brain. J Neurosci 24, 3592-3599
40. Gouras, G. K., Tampellini, D., Takahashi, R. H., and Capetillo-Zarate, E. (2010) Intraneuronal betaamyloid accumulation and synapse pathology in Alzheimer's disease. Acta Neuropathol 119, 523-541
41. Spencer, B., Marr, R. A., Gindi, R., Potkar, R., Michael, S., Adame, A., Rockenstein, E., Verma, I. M., and Masliah, E. (2011) Peripheral delivery of a CNS targeted, metaloprotease reduces abeta toxicity in a mouse model of Alzheimer's disease. PloS one 6, e16575
42. Spencer, B., Verma, I., Desplats, P., Morvinski, D., Rockenstein, E., Adame, A., and Masliah, E. (2014) A neuroprotective brain-penetrating endopeptidase fusion protein ameliorates Alzheimer disease pathology and restores neurogenesis. J Biol Chem 289, 17917-17931
43. Maccioni, R. B., Rojo, L. E., Fernandez, J. A., and Kuljis, R. O. (2009) The role of neuroimmunomodulation in Alzheimer's disease. Ann NY Acad Sci 1153, 240-246
44. Simard, A. R., Soulet, D., Gowing, G., Julien, J. P., and Rivest, S. (2006) Bone marrow-derived microglia play a critical role in restricting senile plaque formation in Alzheimer's disease. Neuron 49, 489-502
45. Yan, Q., Zhang, J., Liu, H., Babu-Khan, S., Vassar, R., Biere, A. L., Citron, M., and Landreth, G. (2003) Anti-inflammatory drug therapy alters beta-amyloid processing and deposition in an animal model of Alzheimer's disease. J Neurosci 23, 7504-7509
46. Watanabe-Nakayama, T., Ono, K., Itami, M., Takahashi, R., Teplow, D. B., and Yamada, M. (2016) Highspeed atomic force microscopy reveals structural dynamics of amyloid beta1-42 aggregates. Proc Natl Acad Sci USA 113, 5835-5840
47. Barz, B., Liao, Q., and Strodel, B. (2018) Pathways of Amyloid-beta Aggregation Depend on Oligomer Shape. J Am Chem Soc 140, 319-327
48. Yang, T., Li, S., Xu, H., Walsh, D. M., and Selkoe, D. J. (2017) Large Soluble Oligomers of Amyloid beta-Protein from Alzheimer Brain Are Far Less Neuroactive Than the Smaller Oligomers to Which They Dissociate. J Neurosci 37, 152-163
49. Benilova, I., Karran, E., and De Strooper, B. (2012) The toxic Abeta oligomer and Alzheimer's disease: an emperor in need of clothes. Nat Neurosci 15, 349-357
50. Cline, E. N., Bicca, M. A., Viola, K. L., and Klein, W. L. (2018) The Amyloid-beta Oligomer Hypothesis: Beginning of the Third Decade. J Alzheimers Dis 64, S567-S610
51. Teplow, D. B. (2013) On the subject of rigor in the study of amyloid beta-protein assembly. Alzheimers Res Ther 5, 39
52. Ferreira, S. T., Lourenco, M. V., Oliveira, M. M., and De Felice, F. G. (2015) Soluble amyloid-beta oligomers as synaptotoxins leading to cognitive impairment in Alzheimer's disease. Front Cell Neurosci 9, 191
53. Sengupta, U., Nilson, A. N., and Kayed, R. (2016) The Role of Amyloid-beta Oligomers in Toxicity, Propagation, and Immunotherapy. EBioMedicine 6, 42-49
54. Selkoe, D. J., and Hardy, J. (2016) The amyloid hypothesis of Alzheimer's disease at 25 years. EMBO Mol Med 8, 595-608
55. Beach, T. G., Sue, L. I., Walker, D. G., Roher, A. E., Lue, L., Vedders, L., Connor, D. J., Sabbagh, M. N., and Rogers, J. (2008) The Sun Health Research Institute Brain Donation Program: description and experience, 1987-2007. Cell Tissue Bank 9, 229-245
56. Beach, T. G., Adler, C. H., Sue, L. I., Serrano, G., Shill, H. A., Walker, D. G., Lue, L., Roher, A. E., Dugger, B. N., Maarouf, C., Birdsill, A. C., Intorcia, A., Saxon-Labelle, M., Pullen, J., Scroggins, A., Filon, J., Scott, S., Hoffman, B., Garcia, A., Caviness, J. N., Hentz, J. G., Driver-Dunckley, E., Jacobson, S. A., Davis, K. J., Belden, C. M., Long, K. E., Malek-Ahmadi, M., Powell, J. J., Gale, L. D., Nicholson, L. R., Caselli, R. J., Woodruff, B. K., Rapscak, S. Z., Ahern, G. L., Shi, J., Burke, A. D., Reiman, E. M., and Sabbagh, M. N. (2015) Arizona Study of Aging and Neurodegenerative Disorders and Brain and Body Donation Program. Neuropathology 35, 354-389
57. He, P., Zhong, Z., Lindholm, K., Berning, L., Lee, W., Lemere, C., Staufenbiel, M., Li, R., and Shen, Y (2007) Deletion of tumor necrosis factor death receptor inhibits amyloid beta generation and prevents learning and memory deficits in Alzheimer's mice. J Cell Biol 178, 829-841

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of reducing or inhibiting Aβ toxicity in a subject, or a method of treating, ameliorating, or preventing an Aβ disease or disorder, or a method of slowing cognitive decline in a patient diagnosed with an Aβ disease or disorder, or a method of reducing amyloid plaque load in a subject, or a method of preventing memory loss or cognitive decline in an asymptomatic patient, which comprises administering to the subject or expressing in the subject an effective amount of an antibody that selectively binds to oligomeric Aβ, wherein the antibody is engineered for effective transport across the blood brain barrier (BBB).

2. The method of claim 1, wherein the antibody reduces intracellular Aβ inclusions.

3. The method of claim 1, wherein the antibody reduces or inhibits amyloid accumulation, fibrillary deposits, microgliosis, astrogliosis, and/or dendritic spine loss.

4. The method of claim 1, wherein administering the antibody to a subject increases neurogenesis.

5. The method of any one of claims 1 to 4, wherein the antibody comprises:
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence AISGSGGSTYYADSVKG (SEQ ID NO:4);
(c) a heavy chain complementarity determining region 3 (VH-CDR3) comprising the amino acid sequence SYGSVKISCFDY (SEQ ID NO:6);
(d) a light chain complementarity determining region 1 (VL-CDR1) comprising the amino acid sequence KSSQSVLYNSNNKNYLA (SEQ ID NO:8);
(e) a light chain complementarity determining region 2 (VL-CDR2) comprising the amino acid sequence WASTRES (SEQ ID NO:10); and
(f) a light chain complementarity determining region 3 (VL-CDR3) comprising the amino acid sequence QQFYSTPPT (SEQ ID NO:12);
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence SGSGGS (SEQ ID NO:16);
(c′) a heavy chain complementarity determining region 3 (VH-CDR3) comprising the amino acid sequence SYGSVKISCFDY (SEQ ID NO:6);
(d′) a light chain complementarity determining region 1 (VL-CDR1) comprising the amino acid sequence SQSVLYNSNNKNY (SEQ ID NO:18);
(e′) a light chain complementarity determining region 2 (VL-CDR2) comprising the amino acid sequence WAS (SEQ ID NO:20); and
(f′) a light chain complementarity determining region 3 (VL-CDR3) comprising the amino acid sequence FYSTPP (SEQ ID NO:22).

6. The method of claim 5, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:24.

7. The method of any one of claims 1 to 4, wherein the antibody comprises:
(a) a heavy chain complementarity determining region 1 (VH-CDR1) comprising the amino acid sequence SYPMS (SEQ ID NO:27);
(b) a heavy chain complementarity determining region 2 (VH-CDR2) comprising the amino acid sequence AIQHTGAPTTYADSVKG (SEQ ID NO:29);
(c) a heavy chain complementarity determining region 3 (VH-CDR3) comprising the amino acid sequence AFPPFDY (SEQ ID NO:31);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence SASSLQS (SEQ ID NO:35); and
(f) a light chain complementarity determining region 3 (VL-CDR3) comprising the amino acid sequence QQRETGPKA (SEQ ID NO:37);
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QHTGAP (SEQ ID NO:39);
(c′) a heavy chain complementarity determining region 3 (VH-CDR3) comprising the amino acid sequence AFPPFDY (SEQ ID NO:31);
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence SAS (SEQ ID NO:43); and
(f′) a light chain complementarity determining region 3 (VL-CDR3) comprising the amino acid sequence RETGPK (SEQ ID NO:45).

8. The method of claim 7, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:47.

9. The method of any one of claims 1 to 4, wherein the antibody comprises:
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence SIQPEGRRTAYVDSVK (SEQ ID NO:50);
(c) a heavy chain complementarity determining region 3 (VH-CDR3) comprising the amino acid sequence PPERFDY (SEQ ID NO:52);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence AASSLQS (SEQ ID NO:54); and
(f) a light chain complementarity determining region 3 (VL-CDR3) comprising the amino acid sequence QQSYSTPNT (SEQ ID NO:56);
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QPEGRR (SEQ ID NO:58);
(c′) a heavy chain complementarity determining region 3 (VH-CDR3) comprising the amino acid sequence PPERFDY (SEQ ID NO:52;
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence AAS (SEQ ID NO:60); and
(f′) a light chain complementarity determining region 3 (VL-CDR3) comprising the amino acid sequence SYSTPN (SEQ ID NO:62).

10. The method of claim 9, wherein the antibody comprises a VH amino acid sequence and a VL amino acid sequence at least 70%, at least 75%, at least 80%, at least 85%, at least 90%, at least 95%, at least 96%, at least 97%, at least 98%, at least 99% identical, or identical to SEQ ID NO:64.

11. The method of claim 1, wherein the antibody comprises a scFv, a humanized antibody, a fully human antibody, a chimeric antibody, a single-chain antibody, a diabody, an intrabody, or antigen-binding fragments thereof.

12. The method of claim 1, wherein the antibody comprises or is linked to a polypeptide which binds to a blood brain barrier (BBB) receptor.

13. The method of claim 12, wherein the BBB receptor comprises low-density lipoprotein (LDL) receptor (LDLR), transferrin receptor, or insulin-like growth factor receptor.

14. The method of claim 12, wherein the polypeptide which binds to a BBB comprises an LDLR-binding domain of apoB.

15. The method of claim 1, wherein the antibody comprises or is linked to a cell penetrating peptide (CPP).

16. An antibody engineered for transport across the blood brain barrier (BBB) which comprises:
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence AISGSGGSTYYADSVKG (SEQ ID NO:4);
(c) a heavy chain complementarity determining region 3 (VH-CDR3) comprising the amino acid sequence SYGSVKISCFDY (SEQ ID NO:6);
(d) a light chain complementarity determining region 1 (VL-CDR1) comprising the amino acid sequence KSSQSVLYNSNNKNYLA (SEQ ID NO:8);
(e) a light chain complementarity determining region 2 (VL-CDR2) comprising the amino acid sequence WASTRES (SEQ ID NO:10); and
(f) a light chain complementarity determining region 3 (VL-CDR3) comprising the amino acid sequence QQFYSTPPT (SEQ ID NO:12).
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence SGSGGS (SEQ ID NO:16);
(c′) a heavy chain complementarity determining region 3 (VH-CDR3) comprising the amino acid sequence SYGSVKISCFDY (SEQ ID NO:6);
(d′) a light chain complementarity determining region 1 (VL-CDR1) comprising the amino acid sequence SQSVLYNSNNKNY (SEQ ID NO:18);
(e′) a light chain complementarity determining region 2 (VL-CDR2) comprising the amino acid sequence WAS (SEQ ID NO:20); and
(f′) a light chain complementarity determining region 3 (VL-CDR3) comprising the amino acid sequence FYSTPP (SEQ ID NO:22).

17. An antibody engineered for transport across the blood brain barrier (BBB) which comprises:
(a) a heavy chain complementarity determining region 1 (VH-CDR1) comprising the amino acid sequence SYPMS (SEQ ID NO:27);
(b) a heavy chain complementarity determining region 2 (VH-CDR2) comprising the amino acid sequence AIQHTGAPTTYADSVKG (SEQ ID NO:29);
(c) a heavy chain complementarity determining region 3 (VH-CDR3) comprising the amino acid sequence AFPPFDY (SEQ ID NO:31);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence SASSLQS (SEQ ID NO:35); and
(f) a light chain complementarity determining region 3 (VL-CDR3) comprising the amino acid sequence QQRETGPKA (SEQ ID NO:37);
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QHTGAP (SEQ ID NO:39);
(c′) a heavy chain complementarity determining region 3 (VH-CDR3) comprising the amino acid sequence AFPPFDY (SEQ ID NO:31);
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence SAS (SEQ ID NO:43); and
(f′) a light chain complementarity determining region 3 (VL-CDR3) comprising the amino acid sequence RETGPK (SEQ ID NO:45).

18. An antibody engineered for transport across the blood brain barrier (BBB) which comprises:
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence SIQPEGRRTAYVDSVK (SEQ ID NO:50);
(c) a heavy chain complementarity determining region 3 (VH-CDR3) comprising the amino acid sequence PPERFDY (SEQ ID NO:52);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence AASSLQS (SEQ ID NO:54); and
(f) a light chain complementarity determining region 3 (VL-CDR3) comprising the amino acid sequence QQSYSTPNT (SEQ ID NO:56);
or
wherein the antibody comprises:
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QPEGRR (SEQ ID NO:58);
(c′) a heavy chain complementarity determining region 3 (VH-CDR3) comprising the amino acid sequence PPERFDY (SEQ ID NO:52;
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence AAS (SEQ ID NO:60); and
(f′) a light chain complementarity determining region 3 (VL-CDR3) comprising the amino acid sequence SYSTPN (SEQ ID NO:62).

19. The antibody of any one of claims 16-18, which comprises or is linked to a polypeptide which binds to a blood brain barrier (BBB) receptor.

20. The antibody of claim 19, wherein the BBB receptor comprises low-density lipoprotein (LDL) receptor (LDLR), transferrin receptor, or insulin-like growth factor receptor.

21. The antibody of claim 19, wherein the polypeptide which binds to a BBB receptor comprises an LDLR-binding domain of apoB.

22. The antibody of claim 19, which comprises SEQ ID NO:85, SEQ ID NO:86, or SEQ ID NO:87.

23. The antibody of any one of claims 16-18, wherein the antibody comprises or is linked to a cell penetrating peptide (CPP).

24. A nucleic acid which encodes the antibody of any one of claims 16-18.

25. A nucleic acid which encodes the antibody of claim 19.

26. A nucleic acid which encodes the antibody of claim 23.

27. The nucleic acid of claim 25, which comprises SEQ ID NO:81 or SEQ ID NO:83.

28. The nucleic acid of claim 25 or 27, which comprises DNA, RNA, nucleotide analogs or combinations thereof.

29. An expression vector comprising the nucleic acid of claim 24.

30. An expression vector comprising the nucleic acid of any one of claims 25 to 28.

31. A pharmaceutical composition comprising the antibody of any of claims 16-18 or 20-22.

32. A pharmaceutical composition comprising the antibody of claim 19.

33. A pharmaceutical composition comprising the antibody of claim 23.

34. A pharmaceutical composition comprising the nucleic acid of claim 24.

35. A pharmaceutical composition comprising the nucleic acid of any one of claims 25 to 28.

36. A pharmaceutical composition comprising the nucleic acid of claim 28.

37. A pharmaceutical composition comprising the expression vector of claim 30.

38. A method of determining whether a candidate agent is useful for treating or ameliorating an Aβ disease, which comprises comparing the candidate agent to a control reagent, wherein the control reagent comprises or expresses an antibody engineered for transport across the blood brain barrier (BBB) which comprises:
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence AISGSGGSTYYADSVKG (SEQ ID NO:4);
(c) a heavy chain complementarity determining region 3 (VH-CDR3) comprising the amino acid sequence SYGSVKISCFDY (SEQ ID NO:6);
(d) a light chain complementarity determining region 1 (VL-CDR1) comprising the amino acid sequence KSSQSVLYNSNNKNYLA (SEQ ID NO:8);
(e) a light chain complementarity determining region 2 (VL-CDR2) comprising the amino acid sequence WASTRES (SEQ ID NO:10); and
(f) a light chain complementarity determining region 3 (VL-CDR3) comprising the amino acid sequence QQFYSTPPT (SEQ ID NO:12);
or
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence SGSGGS (SEQ ID NO:16);
(c′) a heavy chain complementarity determining region 3 (VH-CDR3) comprising the amino acid sequence SYGSVKISCFDY (SEQ ID NO:6);
(d′) a light chain complementarity determining region 1 (VL-CDR1) comprising the amino acid sequence SQSVLYNSNNKNY (SEQ ID NO:18);
(e′) a light chain complementarity determining region 2 (VL-CDR2) comprising the amino acid sequence WAS (SEQ ID NO:20); and
(f′) a light chain complementarity determining region 3 (VL-CDR3) comprising the amino acid sequence FYSTPP (SEQ ID NO:22);
or
(a) a heavy chain complementarity determining region 1 (VH-CDR1) comprising the amino acid sequence SYPMS (SEQ ID NO:27);
(b) a heavy chain complementarity determining region 2 (VH-CDR2) comprising the amino acid sequence AIQHTGAPTTYADSVKG (SEQ ID NO:29);
(c) a heavy chain complementarity determining region 3 (VH-CDR3) comprising the amino acid sequence AFPPFDY (SEQ ID NO:31);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence SASSLQS (SEQ ID NO:35); and
(f) a light chain complementarity determining region 3 (VL-CDR3) comprising the amino acid sequence QQRETGPKA (SEQ ID NO:37);
or
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QHTGAP (SEQ ID NO:39);
(c′) a heavy chain complementarity determining region 3 (VH-CDR3) comprising the amino acid sequence AFPPFDY (SEQ ID NO:31);
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence SAS (SEQ ID NO:43); and
(f) a light chain complementarity determining region 3 (VL-CDR3) comprising the amino acid sequence RETGPK (SEQ ID NO:45);
or
(a) a heavy chain complementarity determining region 1 (VH-CDR1) comprising the amino acid sequence SYAMS (SEQ ID NO:2);
(b) a heavy chain complementarity determining region 2 (VH-CDR2) comprising the amino acid sequence SIQPEGRRTAYVDSVK (SEQ ID NO:50);
(c) a heavy chain complementarity determining region 3 (VH-CDR3) comprising the amino acid sequence PPERFDY (SEQ ID NO:52);
(d) a light chain complementarity determining region 1 (VL-CDR1) comprising the amino acid sequence RASQSISSYLN (SEQ ID NO:33);
(e) a light chain complementarity determining region 2 (VL-CDR2) comprising the amino acid sequence AASSLQS (SEQ ID NO:54); and
(f) a light chain complementarity determining region 3 (VL-CDR3) comprising the amino acid sequence QQSYSTPNT (SEQ ID NO:56);
or
(a′) a heavy chain complementarity determining region 1 (VH-CDR1) comprising the amino acid sequence GFTFSSY (SEQ ID NO:14);
(b′) a heavy chain complementarity determining region 2 (VH-CDR2) comprising the amino acid sequence QPEGRR (SEQ ID NO:58);
(c′) a heavy chain complementarity determining region 3 (VH-CDR3) comprising the amino acid sequence PPERFDY (SEQ ID NO:52;
(d′) a light chain complementarity determining region 1 (VL-CDR1) comprising the amino acid sequence SQSISSY (SEQ ID NO:41);
(e′) a light chain complementarity determining region 2 (VL-CDR2) comprising the amino acid sequence AAS (SEQ ID NO:60); and
(f′) a light chain complementarity determining region 3 (VL-CDR3) comprising the amino acid sequence SYSTPN (SEQ ID NO:62).

39. The method of claim 38, wherein the Aβ disease is Alzheimer's disease.


SEQUENCE LISTING (109 sequences)

SEQ ID NO: 1 - DNA, 15 bp, Artificial Sequence
  Synthetic polynucleotide
  CDS (1)..(15)
agctatgccatgagc

SEQ ID NO: 2 - protein, 5 aa, Artificial Sequence
  Synthetic Construct
SYAMS

SEQ ID NO: 3 - DNA, 51 bp, Artificial Sequence
  Synthetic polynucleotide
  CDS (1)..(51)
gctattagtggtagtggtggtagcacatactacgcagactccgtgaagggc

SEQ ID NO: 4 - protein, 17 aa, Artificial Sequence
  Synthetic Construct
AISGSGGSTYYADSVKG

SEQ ID NO: 5 - DNA, 36 bp, Artificial Sequence
  Synthetic polynucleotide
  CDS (1)..(36)
agctatggttcagttaaaataagctgctttgactac

SEQ ID NO: 6 - protein, 12 aa, Artificial Sequence
  Synthetic Construct
SYGSVKISCFDY

SEQ ID NO: 7 - DNA, 51 bp, Artificial Sequence
  Synthetic polynucleotide
  CDS (1)..(51)
aagtccagccagagtgttctttacaactccaacaataagaactacttagct

SEQ ID NO: 8 - protein, 17 aa, Artificial Sequence
  Synthetic Construct
KSSQSVLYNSNNKNYLA

SEQ ID NO: 9 - DNA, 21 bp, Artificial Sequence
  Synthetic polynucleotide
  CDS (1)..(21)
tgggcatcaacccgggaatcc

SEQ ID NO: 10 - protein, 7 aa, Artificial Sequence
  Synthetic Construct
WASTRES

SEQ ID NO: 11 - DNA, 27 bp, Artificial Sequence
  Synthetic polynucleotide
  CDS (1)..(27)
cagcaattttatagtactcctccgact

SEQ ID NO: 12 - protein, 9 aa, Artificial Sequence
  Synthetic Construct
QQFYSTPPT

SEQ ID NO: 13 - DNA, 21 bp, Artificial Sequence
  Synthetic polynucleotide
  CDS (1)..(21)
ggattcacctttagcagctat

SEQ ID NO: 14 - protein, 7 aa, Artificial Sequence
  Synthetic Construct
GFTFSSY

SEQ ID NO: 15 - DNA, 18 bp, Artificial Sequence
  Synthetic polynucleotide
  CDS (1)..(18)
agtggtagtggtggtagc

SEQ ID NO: 16 - protein, 6 aa, Artificial Sequence
  Synthetic Construct
SGSGGS

SEQ ID NO: 17 - DNA, 39 bp, Artificial Sequence
  Synthetic polynucleotide
  CDS (1)..(39)
agccagagtgttctttacaactccaacaataagaactac

SEQ ID NO: 18 - protein, 13 aa, Artificial Sequence
  Synthetic Construct
SQSVLYNSNNKNY

SEQ ID NO: 19 - DNA, 9 bp, Artificial Sequence
  Synthetic polynucleotide
  CDS (1)..(9)
tgggcatca

SEQ ID NO: 20 - protein, 3 aa, Artificial Sequence
  Synthetic construct
WAS

SEQ ID NO: 21 - DNA, 18 bp, Artificial Sequence
  Synthetic polynucleotide
  CDS (1)..(18)
ttttatagtactcctccg

SEQ ID NO: 22 - protein, 6 aa, Artificial Sequence
  Synthetic Construct
FYSTPP

SEQ ID NO: 23 - DNA, 756 bp, Artificial Sequence
  Synthetic polynucleotide
  CDS (1)..(756)
atggcccaggtacagctgcaggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccgattcaccatctccagagacaattccaagaacacgctgtatctgcaaatgaacagcctgagagctgaggacacggctgtgtattactgtgcgaagagctatggttcagttaaaataagctgctttgactactggggccagagcaccctggtcaccgtctcctcaggtggaggcggttcaggcggaggtggctctggcggtggcggatcggaaattgtgctgacgcagtctccagactccctggctgtgtctctgggcgagagggccaccatcaactgcaagtccagccagagtgttctttacaactccaacaataagaactacttagcttggtaccagcagaaaccaggacagtctcctgagttgctcatttactgggcatcaacccgggaatccggggtccctgaccgattcagtggcagcgggtctgggacagaattcactcttaccatcagcagcctgcaggctgaggatgtggcagtttattactgtcagcaattttatagtactcctccgacttttggccaggggaccaagctggagatcaaacgt

SEQ ID NO: 24 - protein, 252 aa, Artificial Sequence
  Synthetic Construct
MAQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGSVKISCFDYWGQSTLVTVSSGGGGSGGGGSGGGGSEIVLTQSPDSLAVSLGERATINCKSSQSVLYNSNNKNYLAWYQQKPGQSPELLIYWASTRESGVPDRFSGSGSGTEFTLTISSLQAEDVAVYYCQQFYSTPPTFGQGTKLEIKR

SEQ ID NO: 25 - protein, 278 aa, Artificial Sequence
  Synthetic peptide
MAQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGSVKISCFDYWGQSTLVTVSSGGGGSGGGGSGGGGSEIVLTQSPDSLAVSLGERATINCKSSQSVLYNSNNKNYLAWYQQKPGQSPELLIYWASTRESGVPDRFSGSGSGTEFTLTISSLQAEDVAVYYCQQFYSTPPTFGQGTKLEIKRAAAHHHHHHGAAEQKLISEEDLNGAA

SEQ ID NO: 26 - DNA, 15 bp, Artificial Sequence
  Synthetic construct
  CDS (1)..(15)
agctatcccatgagc

SEQ ID NO: 27 - protein, 5 aa, Artificial Sequence
  Synthetic Construct
SYPMS

SEQ ID NO: 28 - DNA, 51 bp, Artificial Sequence
  Synthetic construct
  CDS (1)..(51)
gcgattcagcatactggtgcgccgacaacttacgcagactccgtgaagggc

SEQ ID NO: 29 - protein, 17 aa, Artificial Sequence
  Synthetic Construct
AIQHTGAPTTYADSVKG

SEQ ID NO: 30 - DNA, 21 bp, Artificial Sequence
  Synthetic construct
  CDS (1)..(21)
gcgtttccgccgtttgactac

SEQ ID NO: 31 - protein, 7 aa, Artificial Sequence
  Synthetic Construct
AFPPFDY

SEQ ID NO: 32 - DNA, 33 bp, Artificial Sequence
  Synthetic construct
  CDS (1)..(33)
cgggcaagtcagagcattagcagctatttaaat

SEQ ID NO: 33 - protein, 11 aa, Artificial Sequence
  Synthetic Construct
RASQSISSYLN

SEQ ID NO: 34 - DNA, 21 bp, Artificial Sequence
  Synthetic construct
  CDS (1)..(21)
tctgcatcctctttgcaaagt

SEQ ID NO: 35 - protein, 7 aa, Artificial Sequence
  Synthetic Construct
SASSLQS

SEQ ID NO: 36 - DNA, 27 bp, Artificial Sequence
  Synthetic construct
  CDS (1)..(27)
caacagcgggagactgggcctaaggcg

SEQ ID NO: 37 - protein, 9 aa, Artificial Sequence
  Synthetic Construct
QQRETGPKA

SEQ ID NO: 38 - DNA, 18 bp, Artificial Sequence
  Synthetic construct
  CDS (1)..(18)
cagcatactggtgcgccg

SEQ ID NO: 39 - protein, 6 aa, Artificial Sequence
  Synthetic Construct
QHTGAP

SEQ ID NO: 40 - DNA, 21 bp, Artificial Sequence
  Synthetic construct
  CDS (1)..(21)
agtcagagcattagcagctat

SEQ ID NO: 41 - protein, 7 aa, Artificial Sequence
  Synthetic Construct
SQSISSY

SEQ ID NO: 42 - DNA, 9 bp, Artificial Sequence
  Synthetic construct
  CDS (1)..(9)
tctgcatcc

SEQ ID NO: 43 - protein, 3 aa, Artificial Sequence
  Synthetic peptide
SAS

SEQ ID NO: 44 - DNA, 18 bp, Artificial Sequence
  Synthetic construct
  CDS (1)..(18)
cgggagactgggcctaag

SEQ ID NO: 45 - protein, 6 aa, Artificial Sequence
  Synthetic Construct
RETGPK

SEQ ID NO: 46 - DNA, 726 bp, Artificial Sequence
  Synthetic polynucleotide
  CDS (1)..(726)
atggccgaggtgcagctgttggagtctgggggaggcttggtacagcctggggggtccctgagactctcctgtgcagcctctggattcacctttagcagctatcccatgagctgggtccgccaggctccagggaaggggctggagtgggtctcagcgattcagcatactggtgcgccgacaacttacgcagactccgtgaagggccggttcaccatctccagagacaattccaagaacacgctgtatctgcaaatgaacagcctgagagccgaggacacggccgtatattactgtgcgaaagcgtttccgccgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctattctgcatcctctttgcaaagtggggtcccatcaaggttcagtggcagtggatctgggacagatttcactctcaccatcagcagtctgcaacctgaagattttgcaacttactactgtcaacagcgggagactgggcctaaggcgttcggccaagggaccaaggtggaaatcaaacgg

SEQ ID NO: 47 - protein, 242 aa, Artificial Sequence
  Synthetic Construct
MAEVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPKAFGQGTKVEIKR

SEQ ID NO: 48 - protein, 268 aa, Artificial Sequence
  Synthetic peptide
MAEVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPKAFGQGTKVEIKRAAAHHHHHHGAAEQKLISEEDLNGAA

SEQ ID NO: 49 - DNA, 51 bp, Artificial Sequence
  Synthetic polynucleotide
  CDS (1)..(51)
tctattcagcctgagggtaggcggacagcgtacgtagactccgtgaagggc

SEQ ID NO: 50 - protein, 17 aa, Artificial Sequence
  Synthetic Construct
SIQPEGRRTAYVDSVKG

SEQ ID NO: 51 - DNA, 21 bp, Artificial Sequence
  Synthetic polynucleotide
  CDS (1)..(21)
ccgccggagaggtttgactac

SEQ ID NO: 52 - protein, 7 aa, Artificial Sequence
  Synthetic Construct
PPERFDY

SEQ ID NO: 53 - DNA, 21 bp, Artificial Sequence
  Synthetic polynucleotide
  CDS (1)..(21)
gctgcatccagtttgcaaagt

SEQ ID NO: 54 - protein, 7 aa, Artificial Sequence
  Synthetic Construct
AASSLQS

SEQ ID NO: 55 - DNA, 27 bp, Artificial Sequence
  Synthetic polynucleotide
  CDS (1)..(27)
gaacagagttacagtacccctaatacg

SEQ ID NO: 56 - protein, 9 aa, Artificial Sequence
  Synthetic Construct
EQSYSTPNT

SEQ ID NO: 57 - DNA, 18 bp, Artificial Sequence
  Synthetic polynucleotide
  CDS (1)..(18)
cagcctgagggtaggcgg

SEQ ID NO: 58 - protein, 6 aa, Artificial Sequence
  Synthetic Construct
QPEGRR

SEQ ID NO: 59 - DNA, 9 bp, Artificial Sequence
  Synthetic polynucleotide
  CDS (1)..(9)
gctgcatcc

SEQ ID NO: 60 - protein, 3 aa, Artificial Sequence
  Synthetic construct
AAS

SEQ ID NO: 61 - DNA, 18 bp, Artificial Sequence
  Synthetic polynucleotide
  CDS (1)..(18)
agttacagtacccctaat

SEQ ID NO: 62 - protein, 6 aa, Artificial Sequence
  Synthetic Construct
SYSTPN

SEQ ID NO: 63 - DNA, 726 bp, Artificial Sequence
  Synthetic polynucleotide
  CDS (1)..(726)
atggccgaggtgcagctgttggagtctgggggaggcttggtacagcctggggggtccctgagactctcctgtgcagcctctggattcacctttagcagctatgccatgagctgggtccgccaggctccagggaaggggctggagtgggtctcatctattcagcctgagggtaggcggacagcgtacgtagactccgtgaagggccggttcaccatctccagagacaattccaagaacacgctgtatctacaaatgaacagcctgagagccgaggacacggccgtatattactgtgcgaaaccgccggagaggtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctaatacgttcggccaagggaccaaggtggaaatcaaacgg

SEQ ID NO: 64 - protein, 242 aa, Artificial Sequence
  Synthetic Construct
MAEVQLLESGGGLVQPGGSLRLSCAASGFTFSSYAMSWVRQAPGKGLEWVSSIQPEGRRTAYVDSVKGRFTISRDNSKNTLYLQMNSLRAEDTAVYYCAKPPERFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 65 - DNA, 117 bp, Artificial Sequence
  Synthetic polynucleotide
  CDS (1)..(117)
tcatctgtcattgatgcactccaatacaaattagagggcaccacaagattgacaagaaagagaggattgaagttagccacagctctgtctctgagcaacaaatttgtggagggtagt

SEQ ID NO: 66 - protein, 39 aa, Artificial Sequence
  Synthetic Construct
SSVIDALQYKLEGTTRLTRKRGLKLATALSLSNKFVEGS

SEQ ID NO: 67 - protein, 12 aa, Artificial Sequence
  Synthetic peptide
HPWCCGLRLDLR

SEQ ID NO: 68 - protein, 12 aa, Artificial Sequence
  Synthetic peptide
YHFNGCEDPLCR

SEQ ID NO: 69 - protein, 16 aa, Artificial Sequence
  Synthetic polypeptide
RQIKIWFQNRRMKWKK

SEQ ID NO: 70 - DNA, 81 bp, Artificial Sequence
  Synthetic polynucletide
  CDS (1)..(81)
cagagcctggcccaggaactgggcctgaacgaacgacagatcaaaatctggttccagaaccgccgcatgaagtggaaaaaa

SEQ ID NO: 71 - protein, 27 aa, Artificial Sequence
  Synthetic Construct
QSLAQELGLNERQIKIWFQNRRMKWKK

SEQ ID NO: 72 - protein, 11 aa, Artificial Sequence
  Synthetic peptide
YGRKKRRQRRR

SEQ ID NO: 73 - protein, 13 aa, Artificial Sequence
  Synthetic peptide
GRKKRRQRRRPPQ

SEQ ID NO: 74 - protein, 19 aa, Artificial Sequence
  Synthetic polypeptide
TFFYGGCRGKRNNFKTEEY

SEQ ID NO: 75 - protein, 19 aa, Artificial Sequence
  Synthetic polypeptide
TFFYGGSRGKRNNFKTEEY

SEQ ID NO: 76 - protein, 27 aa, Artificial Sequence
  Synthetic polypeptide
CLIKKALAALAKLNIKLLYGASNLTWG

SEQ ID NO: 77 - protein, 13 aa, Artificial Sequence
  Synthetic polypeptide
GGHKAKGPRKLGS

SEQ ID NO: 78 - protein, 12 aa, Artificial Sequence
  Synthetic polypeptide
THRPPMWSPVWP

SEQ ID NO: 79 - protein, 7 aa, Artificial Sequence
  Synthetic polypeptide
HAIYPRH

SEQ ID NO: 80 - protein, 20 aa, Artificial Sequence
  Synthetic polypeptide
METDTLLLWVLLLWVPGSTG

SEQ ID NO: 81 - DNA, 1015 bp, Artificial Sequence
  Synthetic polynucleotide
  misc_feature (13)..(19): Kozak
  CDS (20)..(88): IgGk signal + SacI
  CDS (89)..(205): ApoB
  CDS (206)..(223): linker
  CDS (224)..(979): C6T-scFv
  CDS (980)..(988): NotI site
  CDS (989)..(1012): FLAG
gtcgacgccgccacaatggatggagacagacacactcctgctatgggtactgctgctctgggttccaggttccactggtgacgagctctcatctgtcattgatgcactccaatacaaattagagggcaccacaagattgacaagaaagagaggattgaagttagccacagctctgtctctgagcaacaaatttgtggagggtagtggtggaggcggatctgccatggcccaggtacagctgcaggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccgattcaccatctccagagacaattccaagaacacgctgtatctgcaaatgaacagcctgagagctgaggacacggctgtgtattactgtgcgaagagctatggttcagttaaaataagctgctttgactactggggccagagcaccctggtcaccgtctcctcaggtggaggcggttcaggcggaggtggctctggcggtggcggatcggaaattgtgctgacgcagtctccagactccctggctgtgtctctgggcgagagggccaccatcaactgcaagtccagccagagtgttctttacaactccaacaataagaactacttagcttggtaccagcagaaaccaggacagtctcctgagttgctcatttactgggcatcaacccgggaatccggggtccctgaccgattcagtggcagcgggtctgggacagaattcactcttaccatcagcagcctgcaggctgaggatgtggcagtttattactgtcagcaattttatagtactcctccgacttttggccaggggaccaagctggagatcaaacgtgcggccgcagattacaaggatgacgatgacaagtga

SEQ ID NO: 82 - protein, 331 aa, Artificial Sequence
  Synthetic Construct
METDTLLLWVLLLWVPGSTGDELSSVIDALQYKLEGTTRLTRKRGLKLATALSLSNKFVEGSGGGGSAMAQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGSVKISCFDYWGQSTLVTVSSGGGGSGGGGSGGGGSEIVLTQSPDSLAVSLGERATINCKSSQSVLYNSNNKNYLAWYQQKPGQSPELLIYWASTRESGVPDRFSGSGSGTEFTLTISSLQAEDVAVYYCQQFYSTPPTFGQGTKLEIKRAAADYKDDDDK

SEQ ID NO: 83 - DNA, 979 bp, Artificial Sequence
  Synthetic polynucleotide
  misc_feature (7)..(13): Kozak
  CDS (14)..(82): IgGk + SacI
  CDS (83)..(199): ApoB
  CDS (200)..(217): linker
  CDS (218)..(943): A4-scFv
  CDS (944)..(952): NotI
  CDS (953)..(976): FLAG
gccgccacaatggatggagacagacacactcctgctatgggtactgctgctctgggttccaggttccactggtgacgagctctcatctgtcattgatgcactccaatacaaattagagggcaccacaagattgacaagaaagagaggattgaagttagccacagctctgtctctgagcaacaaatttgtggagggtagtggtggaggcggatctgccatggccgaggtgcagctgttggagtctgggggaggcttggtacagcctggggggtccctgagactctcctgtgcagcctctggattcacctttagcagctatcccatgagctgggtccgccaggctccagggaaggggctggagtgggtctcagcgattcagcatactggtgcgccgacaacttacgcagactccgtgaagggccggttcaccatctccagagacaattccaagaacacgctgtatctgcaaatgaacagcctgagagccgaggacacggccgtatattactgtgcgaaagcgtttccgccgtttgactactggggccagggaaccctggtcaccgtctcgagcggtggaggcggttcaggcggaggtggcagcggcggtggcgggtcgacggacatccagatgacccagtctccatcctccctgtctgcatctgtaggagacagagtcaccatcacttgccgggcaagtcagagcattagcagctatttaaattggtatcagcagaaaccagggaaagcccctaagctcctgatctattctgcatcctctttgcaaagtggggtcccatcaaggttcagtggcagtggatctgggacagatttcactctcaccatcagcagtctgcaacctgaagattttgcaacttactactgtcaacagcgggagactgggcctaaggcgttcggccaagggaccaaggtggaaatcaaacgggcggccgcagattacaaggatgacgatgacaagtga

SEQ ID NO: 84 - protein, 321 aa, Artificial Sequence
  Synthetic Construct
METDTLLLWVLLLWVPGSTGDELSSVIDALQYKLEGTTRLTRKRGLKLATALSLSNKFVEGSGGGGSAMAEVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPKAFGQGTKVEIKRAAADYKDDDDK

SEQ ID NO: 85 - protein, 297 aa, Artificial Sequence
  Synthetic peptide
SSVIDALQYKLEGTTRLTRKRGLKLATALSLSNKFVEGSGGGGSAMAQVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGSVKISCFDYWGQSTLVTVSSGGGGSGGGGSGGGGSEIVLTQSPDSLAVSLGERATINCKSSQSVLYNSNNKNYLAWYQQKPGQSPELLIYWASTRESGVPDRFSGSGSGTEFTLTISSLQAEDVAVYYCQQFYSTPPTFGQGTKLEIKR

SEQ ID NO: 86 - protein, 286 aa, Artificial Sequence
  Synthetic peptide
SSVIDALQYKLEGTTRLTRKRGLKLATALSLSNKFVEGSGGGGSAMAEVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPAFGQGTKVEIKR

SEQ ID NO: 87 - protein, 287 aa, Artificial Sequence
  Synthetic peptide
SSVIDALQYKLEGTTRLTRKRGLKLATALSLSNKFVEGSGGGGSAMAEVQLLESGGGLVQPGGSLRLSCAASGFTFSSYAMSWVRQAPGKGLEWVSSIQPEGRRTAYVDSVKGRFTISRDNSKNTLYLQMNSLRAEDTAVYYCAKPPERFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR

SEQ ID NO: 88 - protein, 18 aa, Artificial Sequence
  Synthetic peptide
CNCKAPETALCARRCQQH

SEQ ID NO: 89 - protein, 8 aa, Artificial Sequence
  Synthetic peptide
KAPETALD

SEQ ID NO: 90 - protein, 14 aa, Artificial Sequence
  Synthetic peptide
PTVIHGKREVTLHL

SEQ ID NO: 91 - protein, 20 aa, Artificial Sequence
  Synthetic peptide
ELKHFEAKIEKHNHYQKQLE

SEQ ID NO: 92 - protein, 15 aa, Artificial Sequence
  Synthetic peptide
GGGGYGRKKRRQRRR

SEQ ID NO: 93 - protein, 20 aa, Artificial Sequence
  Synthetic peptide
RGFFVLKGRRRRQRRKKRGY

SEQ ID NO: 94 - protein, 18 aa, Artificial Sequence
  Synthetic peptide
RGGRLSYSRRRFSTSTGR

SEQ ID NO: 95 - protein, 7 aa, Artificial Sequence
  Synthetic peptide
MPRLRGC

SEQ ID NO: 96 - protein, 31 aa, Artificial Sequence
  Synthetic peptide
YQQVLTSLPSQNVLQIANDLENLRDLLHLLC

SEQ ID NO: 97 - protein, 12 aa, Artificial Sequence
  Synthetic peptide
TGNYKALHPHNG

SEQ ID NO: 98 - protein, 14 aa, Artificial Sequence
  Synthetic peptide
KKRTLRKNDRKKRC

SEQ ID NO: 99 - protein, 20 aa, Artificial Sequence
  Synthetic peptide
LRKLRKRLLRLRKLRKRLLR

SEQ ID NO: 100 - protein, 29 aa, Artificial Sequence
  Synthetic peptide
YTIWMPENPRPGTPCDIFTNSRGKRASNG

SEQ ID NO: 101 - protein, 13 aa, Artificial Sequence
  Synthetic peptide
HLNILSTLWKYRC

SEQ ID NO: 102 - protein, 12 aa, Artificial Sequence
  Synthetic peptide
PWVPSWMPPRHT

SEQ ID NO: 103 - protein, 16 aa, Artificial Sequence
  Synthetic peptide
FKESWREARGTRIERG

SEQ ID NO: 104 - protein, 9 aa, Artificial Sequence
  Synthetic peptide
CRTIGPSVC

SEQ ID NO: 105 - protein, 12 aa, Artificial Sequence
  Synthetic peptide
RLSSVDSDLSGC

SEQ ID NO: 106 - protein, 4 aa, Artificial Sequence
  Synthetic peptide
CAQK

SEQ ID NO: 107 - protein, 250 aa, Artificial Sequence
  Synthetic peptide
QVQLQESGGGLVQPGGSLRLSCAASGFTFSSYAMSWVRQAPGKGLEWVSAISGSGGSTYYADSVKGRFTISRDNSKNTLYLQMNSLRAEDTAVYYCAKSYGSVKISCFDYWGQSTLVTVSSGGGGSGGGGSGGGGSEIVLTQSPDSLAVSLGERATINCKSSQSVLYNSNNKNYLAWYQQKPGQSPELLIYWASTRESGVPDRFSGSGSGTEFTLTISSLQAEDVAVYYCQQFYSTPPTFGQGTKLEIKR

SEQ ID NO: 108 - protein, 239 aa, Artificial Sequence
  Synthetic peptide
EVQLLESGGGLVQPGGSLRLSCAASGFTFSSYPMSWVRQAPGKGLEWVSAIQHTGAPTTYADSVKGRFTISRDNSKNTLYLQMNSLRAEDTAVYYCAKAFPPFDYWGQGTLVTVSSGGGGSGGGGSGGGGSTDIQMTQSPSSLSASVGDRVTITCRASQSISSYLNWYQQKPGKAPKLLIYSASSLQSGVPSRFSGSGSGTDFTLTISSLQPEDFATYYCQQRETGPAFGQGTKVEIKR

SEQ ID NO: 109 - protein, 240 aa, Artificial Sequence
  Synthetic peptide
EVQLLESGGGLVQPGGSLRLSCAASGFTFSSYAMSWVRQAPGKGLEWVSSIQPEGRRTAYVDSVKGRFTISRDNSKNTLYLQMNSLRAEDTAVYYCAKPPERFDYWGQGTLVTVSSGGGGSGGGGSGGGGSTDIQMTQSPSSLSASVGDRVTITCRASQSISSYLNWYQQKPGKAPKLLIYAASSLQSGVPSRFSGSGSGTDFTLTISSLQPEDFATYYCQQSYSTPNTFGQGTKVEIKR